SEQUENCE LISTING (45 sequences)

SEQ ID NO: 1 - DNA, 20 bp, Artificial Sequence
  An artificially synthesized primer sequence for RT-PCR
gcaaattccatggcaccgtc

SEQ ID NO: 2 - DNA, 19 bp, Artificial Sequence
  An artificially synthesized primer sequence for RT-PCR
tcgccccacttgattttgg

SEQ ID NO: 3 - DNA, 20 bp, Artificial Sequence
  An artificially synthesized primer sequence for RT-PCR
tgggaacaagagggcatctg

SEQ ID NO: 4 - DNA, 22 bp, Artificial Sequence
  An artificially synthesized primer sequence for RT-PCR
ccaccactgcatcaaattcatg

SEQ ID NO: 5 - DNA, 24 bp, Artificial Sequence
  An artificially synthesized primer sequence for RT-PCR
atgtcaagaattccagcttcttcc

SEQ ID NO: 6 - DNA, 24 bp, Artificial Sequence
  An artificially synthesized primer sequence for RT-PCR
caattttatctttgaaagtaaagg

SEQ ID NO: 7 - DNA, 24 bp, Artificial Sequence
  An artificially synthesized primer sequence for RT-PCR
agaaatcattgcaagcggctggag

SEQ ID NO: 8 - DNA, 24 bp, Artificial Sequence
  An artificially synthesized primer sequence for RT-PCR
ctggctccttatcttttttaatgg

SEQ ID NO: 9 - DNA, 25 bp, Artificial Sequence
  An artificially synthesized primer sequence for RT-PCR
tatgggctgccttacgtggtgcgtg

SEQ ID NO: 10 - DNA, 25 bp, Artificial Sequence
  An artificially synthesized primer sequence for RT-PCR
cgggatcaggatggggcctggggtc

SEQ ID NO: 11 - DNA, 25 bp, Artificial Sequence
  An artificially synthesized primer sequence for RT-PCR
gcaggccctcgccttcgcccccttc

SEQ ID NO: 12 - DNA, 24 bp, Artificial Sequence
  An artificially synthesized primer sequence for RT-PCR
tccggcctgtcacagctcttcacc

SEQ ID NO: 13 - DNA, 21 bp, Artificial Sequence
  An artificially synthesized sense strand sequence for siRNA
gcagcacgacuucuucaagtt

SEQ ID NO: 14 - DNA, 21 bp, Artificial Sequence
  An artificially synthesized antisense strand sequence for siRNA
cuugaagaagucgugcugctt

SEQ ID NO: 15 - DNA, 21 bp, Artificial Sequence
  An artificially synthesized sense strand sequence for siRNA
gaguucugcgaguguuacatt

SEQ ID NO: 16 - DNA, 21 bp, Artificial Sequence
  An artificially synthesized antisense strand sequence for siRNA
uguaacacucgcagaacuctt

SEQ ID NO: 17 - DNA, 21 bp, Artificial Sequence
  An artificially synthesized sense strand sequence for siRNA
gaaauuauucaaagaacautt

SEQ ID NO: 18 - DNA, 21 bp, Artificial Sequence
  An artificially synthesized antisense strand sequence for siRNA
auguucuuugaauaauuuctt

SEQ ID NO: 19 - DNA, 21 bp, Artificial Sequence
  An artificially synthesized sense strand sequence for siRNA
ggaucugagcccugacccutt

SEQ ID NO: 20 - DNA, 21 bp, Artificial Sequence
  An artificially synthesized antisense strand sequence for siRNA
agggucagggcucagaucctt

SEQ ID NO: 21 - DNA, 21 bp, Artificial Sequence
  An artificially synthesized sense strand sequence for siRNA
gcauagcucugacccuggatt

SEQ ID NO: 22 - DNA, 21 bp, Artificial Sequence
  An artificially synthesized antisense strand sequence for siRNA
uccagggucagagcuaugctt

SEQ ID NO: 23 - DNA, 4562 bp, Homo sapiens
ggtgctgcggcccgcgccgccatcttggattttactctccatttttctctggaattatttttggtgattaattttctgggggggactgggacgcggggcccggcggcgcggccccgcatcgcagcggccgggcagcggggcctgggacgcgccccgaggaggagcggggcggcgcaggcggagagaacattgaaagtattctctaagctatttgaagagagtgactaaatgcacctgggtcaggctgtctgtgggtatgaagtggttgggagaatccaagaacatggtggtgaatggcaggagaaatggaggcaagttgtctaatgaccatcagcagaatcaatcaaaattacagcacacggggaaggacaccctgaaggctggcaaaaatgcagtcgagaggaggtcgaacagatgtaatggtaactcgggatttgaaggacagagtcgctatgtaccatcctctggaatgtccgccaaggaactctgtgaaaatgatgacctagcaaccagtttggttcttgatccctatttaggttttcaaacacacaaaatgaatactagcgcctttccttcgaggagctcaaggcatttttcaaaatctgacagtttttctcacaacaaccctgtgagatttaggcctattaaaggaaggcaggaagaactaaaggaagtaattgaacgttttaagaaagatgaacacttggagaaagccttcaaatgtttgacttcaggcgaatgggcacggcactattttctcaacaagaataaaatgcaggagaaattattcaaagaacatgtatttatttatttgcgaatgtttgcaactgacagtggatttgaaatattgccatgtaatagatactcatcagaacaaaatggagccaaaatagttgcaacaaaagagtggaaacgaaatgacaaaatagaattactggtgggttgtattgccgaactttcagaaattgaggagaacatgctacttagacatggagaaaacgacttcagtgtcatgtactccacaaggaaaaactgtgctcaactctggctgggtcctgctgcgtttataaaccatgattgcagacctaattgtaagtttgtgtcaactggtcgagatacagcatgtgtgaaggctctaagagacattgaacctggagaagaaatttcttgttattatggagatgggttctttggagaaaataatgagttctgcgagtgttacacttgcgaaagacggggcactggtgcttttaaatccagagtgggactgcctgcgcctgctcctgttatcaatagcaaatatggactcagagaaacagataaacgtttaaataggcttaaaaagttaggtgacagcagcaaaaattcagacagtcaatctgtcagctctaacactgatgcagataccactcaggaaaaaaacaatgcaacttctaaccgaaaatcttcagttggcgtaaaaaagaatagcaagagcagaacgttaacgaggcaatctatgtcaagaattccagcttcttccaactctacctcatctaagctaactcatataaataattccagggtaccaaagaaactgaagaagcctgcaaagcctttactttcaaagataaaattgagaaatcattgcaagcggctggagcaaaagaatgcttcaagaaaactcgaaatgggaaacttagtactgaaagagcctaaagtagttctgtataaaaatttgcccattaaaaaagataaggagccagagggaccagcccaagccgcagttgccagcgggtgcttgactagacacgcggcgagagaacacagacagaatcctgtgagaggtgctcattcgcagggggagagctcgccctgcacctacataactcggcggtcagtgaggacaagaacaaatctgaaggaggcctctgacatcaagcttgaaccaaatacgttgaatggctataaaagcagtgtgacggaaccttgccccgacagtggtgaacagctgcagccagctcctgtgctgcaggaggaagaactggctcatgagactgcacaaaaaggggaggcaaagtgtcataagagtgacacaggcatgtccaaaaagaagtcacgacaaggaaaacttgtgaaacagtttgcaaaaatagaggaatctactccagtgcacgattctcctggaaaagacgacgcggtaccagatttgatgggtccccattctgaccagggtgagcacagtggcactgtgggcgtgcctgtgagctacacagactgtgctccttcacccgtcggttgttcagttgtgacatcagatagcttcaaaacaaaagacagctttagaactgcaaaaagtaaaaagaagaggcgaatcacaaggtatgatgcacagttaatcctagaaaataactctgggattcccaaattgactcttcgtaggcgtcatgatagcagcagcaaaacaaatgaccaagagaatgatggaatgaactcttccaaaataagcatcaagttaagcaaagaccatgacaacgataacaatctctatgtagcaaagcttaataatggatttaactcaggatcaggcagtagttctacaaaattaaaaatccagctaaaacgagatgaggaaaatagggggtcttatacagaggggcttcatgaaaatggggtgtgctgcagtgatcctctttctctcttggagtctcgaatggaggtggatgactatagtcagtatgaggaagaaagtacagatgattcctcctcttctgagggcgatgaagaggaggatgactatgatgatgactttgaagacgattttattcctcttcctccagctaagcgcttgaggttaatagttggaaaagactctatagatattgacatttcttcaaggagaagagaagatcagtctttaaggcttaatgcctaagctcttggtcttaacttgacctgggataactactttaaagaaataaaaaattccagtcaattattcctcaactgaaagtttagtggcagcacttctattgtcccttcacttatcagcatactattgtagaaagtgtacagcatactgactcaattcttaagtctgatttgtgcaaatttttatcgtactttttaaatagccttcttacgtgcaattctgagttagaggtaaagccctgttgtaaaataaaggctcaagcaaaattgtacagtgatagcaactttccacacaggacgttgaaaacagtaatgtggctacacagtttttttaactgtaagagcatcagctggctctttaatatatgactaaacaataatttaaaacaaatcatagtagcagcatattaagggtttctagtatgctaatatcaccagcaatgatctttggctttttgatttatttgctagatgtttcccccttggagttttgtcagtttcacactgtttgctggcccaggtgtactgtttgtggcctttgttaatatcgcaaaccattggttgggagtcagattggtttcttaaaaaaaaaaaaaaaatgacatacgtgacagctcacttttcagttcattatatgtacgagggtagcagtgtgtgggatgaggttcgatacagcgtatttattgcttgtcatgtaaattaaaaaccttgtatttaactcttttcaatccttttagataaaattgttctttgcaagaatgattggtgcttattttttcaaaaatttgctgtgaacaacgtgatgacaacaagcaacatttatctaatgaactacagctatcttaatttggttcttcaagttttctgttgcacttgtaaaatgctacaaggaatattaaaaaaatctattcactttaacttataatagtttatgaaataaaaacatgagtcacagcttttgttctgtggtaacctataaaaaaagtttgtctttgagattcaatgtaaagaactgaaaacaatgtatatgttgtaaatatttgtgtgttgtgagaaatttttgtcataagaaattaaaagaacttaccaggaaggtttttaagtttagaaatattcatgccaataaaataggaaattataaatatatagttttaagcactgcatcagtgggagttcttggcttatgttagtttatgttagtttattatgaaaacatcaaagatttttttgactatattatcagttaaacaaaaaggagtcagatttaatttgttttttgaagcactttgagaaattaattttaattaacttaatgagcaaatttttattactactttatgttcaataccaggttcttttcatttctctggattattttgcaaatcattggacagagaatttgggaatataaatctgtaacaggtgtttgacaccagtaggtctctttatttctgggaaatgtgtacctgtactttctgatatacagtgttcctaagtaaaaatcaattcaggggatttgtatagtgtctataggaaagtagcccatgtcttgaaatatgaaaaggaatctgaaggtcatgaaaagtccagtggagaaaatctcaatgcttactgttactactaattgattcctactagtttccaggtttggg

SEQ ID NO: 24 - protein, 885 aa, Homo sapiens
MKWLGESKNMVVNGRRNGGKLSNDHQQNQSKLQHTGKDTLKAGKNAVERRSNRCNGNSGFEGQSRYVPSSGMSAKELCENDDLATSLVLDPYLGFQTHKMNTSAFPSRSSRHFSKSDSFSHNNPVRFRPIKGRQEELKEVIERFKKDEHLEKAFKCLTSGEWARHYFLNKNKMQEKLFKEHVFIYLRMFATDSGFEILPCNRYSSEQNGAKIVATKEWKRNDKIELLVGCIAELSEIEENMLLRHGENDFSVMYSTRKNCAQLWLGPAAFINHDCRPNCKFVSTGRDTACVKALRDIEPGEEISCYYGDGFFGENNEFCECYTCERRGTGAFKSRVGLPAPAPVINSKYGLRETDKRLNRLKKLGDSSKNSDSQSVSSNTDADTTQEKNNATSNRKSSVGVKKNSKSRTLTRQSMSRIPASSNSTSSKLTHINNSRVPKKLKKPAKPLLSKIKLRNHCKRLEQKNASRKLEMGNLVLKEPKVVLYKNLPIKKDKEPEGPAQAAVASGCLTRHAAREHRQNPVRGAHSQGESSPCTYITRRSVRTRTNLKEASDIKLEPNTLNGYKSSVTEPCPDSGEQLQPAPVLQEEELAHETAQKGEAKCHKSDTGMSKKKSRQGKLVKQFAKIEESTPVHDSPGKDDAVPDLMGPHSDQGEHSGTVGVPVSYTDCAPSPVGCSVVTSDSFKTKDSFRTAKSKKKRRITRYDAQLILENNSGIPKLTLRRRHDSSSKTNDQENDGMNSSKISIKLSKDHDNDNNLYVAKLNNGFNSGSGSSSTKLKIQLKRDEENRGSYTEGLHENGVCCSDPLSLLESRMEVDDYSQYEEESTDDSSSSEGDEEEDDYDDDFEDDFIPLPPAKRLRLIVGKDSIDIDISSRRREDQSLRLNA

SEQ ID NO: 25 - DNA, 2711 bp, Homo sapiens
ggtgctgcggcccgcgccgccatcttggattttactctccatttttctctggaattatttttggtgattaattttctgggggggactgggacgcggggcccggcggcgcggccccgcatcgcagcggccgggcagcggggcctgggacgcgccccgaggaggagcggggcggcgcaggcggagagaacattgaaagtattctctaagctatttgaagagagtgactaaatgcacctgggtcaggctgtctgtgggtatgaagtggttgggagaatccaagaacatggtggtgaatggcaggagaaatggaggcaagttgtctaatgaccatcagcagaatcaatcaaaattacagcacacggggaaggacaccctgaaggctggcaaaaatgcagtcgagaggaggtcgaacagatgtaatggtaactcgggatttgaaggacagagtcgctatgtaccatcctctggaatgtccgccaaggaactctgtgaaaatgatgacctagcaaccagtttggttcttgatccctatttaggttttcaaacacacaaaatgaatactagcgcctttccttcgaggagctcaaggcatttttcaaaatctgacagtttttctcacaacaaccctgtgagatttaggcctattaaaggaaggcaggaagaactaaaggaagtaattgaacgttttaagaaagatgaacacttggagaaagccttcaaatgtttgacttcaggcgaatgggcacggcactattttctcaacaagaataaaatgcaggagaaattattcaaagaacatgtatttatttatttgcgaatgtttgcaactgacagtggatttgaaatattgccatgtaatagatactcatcagaacaaaatggagccaaaatagttgcaacaaaagagtggaaacgaaatgacaaaatagaattactggtgggttgtattgccgaactttcagaaattgaggagaacatgctacttagacatggagaaaacgacttcagtgtcatgtactccacaaggaaaaactgtgctcaactctggctgggtcctgctgcgtttataaaccatgattgcagacctaattgtaagtttgtgtcaactggtcgagatacagcatgtgtgaaggctctaagagacattgaacctggagaagaaatttcttgttattatggagatgggttctttggagaaaataatgagttctgcgagtgttacacttgcgaaagacggggcactggtgcttttaaatccagagtgggactgcctgcgcctgctcctgttatcaatagcaaatatggactcagagaaacagataaacgtttaaataggcttaaaaagttaggtgacagcagcaaaaattcagacagtcaatctgtcagctctaacactgatgcagataccactcaggaaaaaaacaatgcaagtaagtaagggagatttgataagcatatcttttaaaagtattttcacacaatttgctttataaagtgtgcttcagtagttttaaacttttaaatactcagagagactgggacttgtgagctttggctgcacttcaaggctctagacgtgatttgagtagaggcacagtctgtatcccatctctaacttcagtaccgtcctctagactatttttcttgaataccttggtaactggatatgagttcttcatcatatgttccaaggtcatcatatgttttaaacattttcaaggtgttagagactgtgatgatgtcgctaagtcctgcaagaagacaaaaggactgagtagaattaaattagactctatacattccagtgcctagccagtttgttagaaaagatgatggacttggggaattcatagcttctggccttaaggcttccaccttttcattgcttgctgacctttttcaaaacgaactgactcagttcagcagaccaccagtaccagactcagaattgtgatagaggagcattttgaacagtgccgtattgtgacatgctgtattggctactccagaaagtaggagtaaagatggaaaggagaaagaagcaacctctgagattccagtggtgtgtgggggcaagatctgatggaaactgaaaaagagaacgaagactaaacaaagagaaaggaaagagaagaaaccctaaatgggcaaaggaaagcacatcctgtttgcggagctttgaaatattggaaccatttctaattgctcctgtttttctgggtaacaccagttttctgtagttgccactaaagcagtagactcttgagtctcacttgtctctgagagagacagaagttagaaagttttgacttggcgattccgaaagtatgcctttgttggcacttaaatgtccagtgagacttcttggcaccttagagccctctgagatactgattattttaggttcttctccctactttcagatgttttcagcccaacactgggtgctctcttccactacagagaatcctgaagaaaagggaaggtgtttcccatgatggtgaatgtcactgccatgaattcctgaatctacctgctgctgggagtcagagtccaagcataacccgtgtagcataaaagcagcgctgtagccctattccagtctttttcgttaatgtccagagtgaacaacaagagttagtcaatcattaactgttgactgttgattctcataataaatgcagcataacgacaaaaaaaaaaaaaaaaaa

SEQ ID NO: 26 - protein, 393 aa, Homo sapiens
MKWLGESKNMVVNGRRNGGKLSNDHQQNQSKLQHTGKDTLKAGKNAVERRSNRCNGNSGFEGQSRYVPSSGMSAKELCENDDLATSLVLDPYLGFQTHKMNTSAFPSRSSRHFSKSDSFSHNNPVRFRPIKGRQEELKEVIERFKKDEHLEKAFKCLTSGEWARHYFLNKNKMQEKLFKEHVFIYLRMFATDSGFEILPCNRYSSEQNGAKIVATKEWKRNDKIELLVGCIAELSEIEENMLLRHGENDFSVMYSTRKNCAQLWLGPAAFINHDCRPNCKFVSTGRDTACVKALRDIEPGEEISCYYGDGFFGENNEFCECYTCERRGTGAFKSRVGLPAPAPVINSKYGLRETDKRLNRLKKLGDSSKNSDSQSVSSNTDADTTQEKNNASK

SEQ ID NO: 27 - DNA, 2318 bp, Homo sapiens
gagcagatgggaggtgcggcgacagtgtttgacgagagccgaaggaggctgtgggaggtgttggcggcggcggcgcgggcgcctgaggaggaggaggagaagcggatgagatcgtggggctcaccagcgtcccccatggcttctgagtagcgtgggagtggagtcagcaccaagccaggctccccgcgcctgccttgccctcacctgctcctgctctctgccagaggcagcatggtccgcagggcaccatggggcccgacagagtgacagcacgagaactgtgcgagaacgacgacctggccaccagcctcgtcctggacccctacctcggtttccgcacccataagatgaacgtcagccctgtgccccccctgcggcgacagcagcacctgcgctcagcgctggaaactttcctgaggcagcgggacctggaggctgcgtaccgggccctgacgctgggaggctggacggcccgctacttccagagccggggcccgcggcaggaggctgccctcaagacccacgtctatcgctacctccgtgccttcctgccggaaagtggctttaccatcctgccctgcacgcgctactccatggagaccaacggggccaagatcgtgtccactcgtgcttggaaaaagaatgagaagctggagctgctggtgggctgcattgcagagctgcgggaggcagatgaggggctgctgagggccggtgagaatgacttcagcatcatgtactcaacccgcaagcggagtgctcagctgtggctgggcccagccgccttcatcaaccatgactgcaaacccaactgcaagtttgtgcctgcagatgggaacgcagcctgcgtgaaggtgctccgggacattgagcctggggacgaggtgacatgcttctacggcgagggcttcttcggcgagaagaatgagcactgtgaatgccacacctgtgagaggaaaggtgaaggagctttccgaaccaggcctagggagcccgcgttgccaccacggcccctggacaagtaccagctgcgtgagaccaagcggcggctgcagcaaggcctggacagtggcagccgacagggcctgctgggccctcgggcctgcgtgcacccatccccgctgcgccgggacccattctgcgccgcctgccagcccctgcgcctgccagcctgcagcgcccgcccagacacctcacccctctggctccagtggctgcctcagccccagccccgagtgcggccccggaagcgccgacgcccccggccccggagggccccagtgctctccacccaccacgctgcccgcgtctccctgcaccgatggggaggctgtggcccccactgccgcctgcgaggagaggccctggtggccctgggccagcccccccacgcccgctgggcccctcagcaggactggcactgggcccggcgctatgggctgccttacgtggtgcgtgtggaccttcgtcgcctggccccagccccaccagctaccccagcccctgctgggaccccaggccccatcctgatcccgaagcaggccctcgccttcgcccccttctccccacccaagcgcctacggctggtggtcagccacggctccatcgacctggatgtcggcggtgaagagctgtgacaggccggacggggaggcccagcagggagagagggtctctctcctagctgctacccaggacctccagaaggagcccttggacctctgggagggagctgacccttgactccagcatagctctgaccctggaatggggttggtttggacacccccagggatctgagccctgaccctttgtgactgctgacccctgagccacccccactcccacagggagccccggccatttgctgccctccccacccctgccccagcctcaggactgcaggagccatccgcccccctcagccccttcctccccagggagcaaagccataaggggcaggggccaccccacggcatctccccagaagtacaggcctcaggaggaggtggaactgatgtagggggtggcactccccagagactgccctcacgaggggactgggttcgctctcagctctgcagctgtctgcggtggggggaaggttggggggtgtctggaggcatgttcccctcaccaccccccgtgggtctcagggaggccgggtgtgacctcatctttctcatggtgctatcctggtgctattggggtggggagctccctcccctcccccacaccagaaaggggtatgttgggggcttggaagcacttgaactttttattttattaaaaccttgttataagcagcaaaaaaaaaa

SEQ ID NO: 28 - protein, 462 aa, Homo sapiens
MGPDRVTARELCENDDLATSLVLDPYLGFRTHKMNVSPVPPLRRQQHLRSALETFLRQRDLEAAYRALTLGGWTARYFQSRGPRQEAALKTHVYRYLRAFLPESGFTILPCTRYSMETNGAKIVSTRAWKKNEKLELLVGCIAELREADEGLLRAGENDFSIMYSTRKRSAQLWLGPAAFINHDCKPNCKFVPADGNAACVKVLRDIEPGDEVTCFYGEGFFGEKNEHCECHTCERKGEGAFRTRPREPALPPRPLDKYQLRETKRRLQQGLDSGSRQGLLGPRACVHPSPLRRDPFCAACQPLRLPACSARPDTSPLWLQWLPQPQPRVRPRKRRRPRPRRAPVLSTHHAARVSLHRWGGCGPHCRLRGEALVALGQPPHARWAPQQDWHWARRYGLPYVVRVDLRRLAPAPPATPAPAGTPGPILIPKQALAFAPFSPPKRLRLVVSHGSIDLDVGGEEL

SEQ ID NO: 29 - RNA, 19 nt, Artificial Sequence
  An artificially synthesized target sequence for siRNA
gaguucugcgaguguuaca

SEQ ID NO: 30 - RNA, 19 nt, Artificial Sequence
  An artificially synthesized target sequence for siRNA
gaaauuauucaaagaacau

SEQ ID NO: 31 - RNA, 19 nt, Artificial Sequence
  An artificially synthesized target sequence for siRNA
ggaucugagcccugacccu

SEQ ID NO: 32 - RNA, 19 nt, Artificial Sequence
  An artificially synthesized target sequence for siRNA
gcauagcucugacccugga

SEQ ID NO: 33 - RNA, 19 nt, Artificial Sequence
  An artificially synthesized target sequence for siRNA
gcagcacgacuucuucaag

SEQ ID NO: 34 - DNA, 21 bp, Artificial Sequence
  An artificially synthesized sense strand sequence for siRNA
gugcgcugcuggugccaactt

SEQ ID NO: 35 - DNA, 21 bp, Artificial Sequence
  An artificially synthesized antisense strand sequence for siRNA
cuugaagaagucgugcugctt

SEQ ID NO: 36 - RNA, 19 nt, Artificial Sequence
  An artificially synthesized target sequence for siRNA
gugcgcugcuggugccaac

SEQ ID NO: 37 - RNA, 19 nt, Artificial Sequence
  An artificially synthesized sense strand sequence for siRNA
auccgcgcgauaguacgua

SEQ ID NO: 38 - RNA, 19 nt, Artificial Sequence
  An artificially synthesized antisense strand sequence for siRNA
uacguacuaucgcgcggau

SEQ ID NO: 39 - RNA, 19 nt, Artificial Sequence
  An artificially synthesized target sequence for siRNA
auccgcgcgauaguacgua

SEQ ID NO: 40 - RNA, 19 nt, Artificial Sequence
  An artificially synthesized sense strand sequence for siRNA
uuacgcguagcguaauacg

SEQ ID NO: 41 - RNA, 19 nt, Artificial Sequence
  An artificially synthesized antisense strand sequence for siRNA
cguauuacgcuacgcguaa

SEQ ID NO: 42 - RNA, 19 nt, Artificial Sequence
  An artificially synthesized target sequence for siRNA
uuacgcguagcguaauacg

SEQ ID NO: 43 - RNA, 19 nt, Artificial Sequence
  An artificially synthesized sense strand sequence for siRNA
uauucgcgcguauagcggu

SEQ ID NO: 44 - RNA, 19 nt, Artificial Sequence
  An artificially synthesized antisense strand sequence for siRNA
accgcuauacgcgcgaaua

SEQ ID NO: 45 - RNA, 19 nt, Artificial Sequence
  An artificially synthesized target sequence for siRNA
uauucgcgcguauagcggu

(19) United States
(12) Patent Application Publication
(10) Pub. No.: US 2013/0203625 A1
(43) Pub. Date: Aug. 8, 2013

(54) SUV420H1 AND SUV420H2 AS TARGET GENES FOR CANCER THERAPY AND DIAGNOSIS

(71) Applicants: Ryuji Hamamoto, Tokyo (JP); Yusuke Nakamura, Tokyo (JP); Takuya Tsunoda, Kanagawa (JP)

(72) Inventors: Ryuji Hamamoto, Tokyo (JP); Yusuke Nakamura, Tokyo (JP); Takuya Tsunoda, Kanagawa (JP)

(73) Assignee: OncoTherapy Science, Inc., Kanagawa (JP)

(21) Appl. No.: 13/817,812

(22) PCT Filed: Aug. 18, 2011
(86) PCT No.: PCT/JP2011/004620

§ 371 Date: Apr. 26, 2013

Related U.S. Application Data
(60) Provisional application No. 61/375,464, filed on Aug. 20, 2010.

(51) Int. Cl. G01N 33/50; C12Q 1/68; C12Q 1/48; G01N 33/68

(52) U.S. Cl. CPC G01N 33/5011; G01N 33/6872; C12Q 1/6897; C12Q 1/48
USPC 506/9; 435/7.1; 435/6.13; 435/15; 435/7.4

(57) ABSTRACT

The present invention relates to the roles played by the SUV420H1 and SUV420H2 genes in carcinogenesis and features a method for treating or preventing cancer by administering a double-stranded molecule against the SUV420H1 or SUV420H2 gene or a composition or vector containing such a double-stranded molecule. The present invention also features methods and kits for detecting or diagnosing cancer in a subject, including detecting an expression level of the SUV420H1 or SUV420H2 gene. The present invention further features methods and kits for assessing or determining the prognosis of a subject with cancer, including detecting the expression level of an SUV420H2 gene. Also, disclosed are methods of screening for candidate substances for treating or preventing cancer or inhibiting cancer cell growth, using as an index their effect on the expression or activity of SUV420H1 or SUV420H2.

### PRIORITY

The present application claims the benefit of U.S. Provisional Application No. 61/375,464, filed on Aug. 20, 2010, the entire contents of which are incorporated by reference herein.

### TECHNICAL FIELD

The present invention relates to the field of biological science, more specifically to the field of cancer research, cancer diagnosis and cancer therapy. In particular, the present invention relates to methods for detecting or diagnosing cancer, or assessing or determining the prognosis of cancer, particularly bladder cancer, cervical cancer, osteosarcoma, lung cancer, soft tissue tumor, breast cancer, chronic myelogenous leukemia (CML), esophageal cancer and gastric cancer as well as methods for treating and preventing disease progression in a subject with cancer, or preventing cancer in a subject, particularly bladder cancer, cervical cancer, osteosarcoma, lung cancer, soft tissue tumor, breast cancer, chronic myelogenous leukemia (CML), esophageal cancer and gastric cancer. Moreover, the present invention relates to methods for screening a candidate substance for either or both of treating and preventing cancer, particularly bladder cancer, cervical cancer, osteosarcoma, lung cancer, soft tissue tumor, breast cancer, chronic myelogenous leukemia (CML), esophageal cancer and gastric cancer.

### BACKGROUND ART

The eukaryotic genome is packaged into chromatin that is an array of nucleosomes in each of which 146 base pairs (bp) of DNA are wrapped around an octamer of core histone proteins −H2A, H2B, H3 and H4 (NPL 1-4). Histone post-translational modifications (PTMs) play key roles in regulating genomic processes, including gene transcription, chromatin assembly, DNA replication, recombination, and DNA repair (NPL 5-7). Diverse, context-specific regulation of these processes has been proposed to be facilitated by interacting regulatory factors that recognize specific histone PTMs or combinations of PTMs, individually or collectively, and direct distinct regulatory outcomes (NPL 8). Lysine residues targeted by methyltransferases (and demethylases) afford the greatest potential combinatorial and functional complexity among histone PTMs because the identity of the site, whether it is unmodified (0m) or mono- (1m), di- (2m), or trimethylated (3m), and the presence of additional PTMs at nearby sites can potentiate the binding of site-specific regulatory factors (NPL 9, 10).

SUV420H1 and SUV420H2 are histone methyltransferases that catalyze di- and trimethylation of histone H4K20 which is characteristic of pericentric heterochromatin. According to current models, H3K9me3 as a result of SUV39H activities stabilizes heterochromatin protein 1 (HP1) binding at heterochromatin (NPL 11, 12), and HP1 proteins then recruit the histone methyltransferases SUV420H1 and SUV420H2 which in turn, trimethylate H4K20 (NPL 13-15).

Previously, it was reported that SMYD3, a histone lysine methyltransferase, stimulates cell proliferation through its methyltransferase activity and plays a crucial role in human carcinogenesis (PL 1, NPL 16-20). Although the research for epigenetics including histone methylation has been advanced, the relationship between abnormal histone methylation (or demethylation) and human carcinogenesis has not been completely clarified yet.

### CITATION LIST

### Patent Literature

[PL1] WO2005/071102

### Non Patent Literature

[NPL1] Luger K, et al., Nature, 1997, 389, 251-260.
[NPL2] Wang G G, et al., Trends Mol Med 2007; 13:363-372.
[NPL3] Wang G G, et al., Trends Mol Med 2007; 13:373-380.
[NPL4] Esteller M, N Engl J Med 2008; 358:1148-1159.
[NPL5] Berger S L, Nature 2007; 447:407-412.
[NPL6] Downs J A, et al., Nature 2007; 447:951-958.
[NPL7] Groth A, et al., Cell 2007; 128:721-733.
[NPL8] Jenuwein T, et al., Science 2001; 293:1074-1080.
[NPL9] Martin C, et al., Nat Rev Mol Cell Biol 2005; 6:838-849.
[NPL10] Ruthenburg A J, et al., Mol Cell 2007; 25:15-30.
[NPL11] Bannister A J, et al., Nature 2001; 410:120-124.
[NPL12] Lachner M, et al., Nature 2001; 410:116-120.
[NPL13] Benetti R, et al., J Cell Biol 2007; 178:925-936.
[NPL14] Kourmouli N, et al., Biochem Biophys Res Commun 2005; 337:901-907.
[NPL15] Schotta G, et al., Genes Dev 2004; 18:1251-1262.
[NPL16] Hamamoto R, et al., Nat Cell Biol 2004; 6:731-40.
[NPL17] Hamamoto R, et al., Cancer Sci 2006; 97:113-8.
[NPL18] Kunizaki M, et al., Cancer Res 2007; 67:10759-65.
[NPL19] Silva F P, et al., Oncogene 2008; 27:2686-92.
[NPL20] Tsuge M, et al., Nat Genet. 2005; 37:1104-7.

### SUMMARY OF INVENTION

The present invention relates to SUV420H1 and SUV420H2, and the roles it plays in cancer carcinogenesis. As such, the present invention relates to novel compositions and methods for detecting, diagnosing, treating and/or preventing cancer, e.g. bladder cancer, cervical cancer, osteosarcoma, lung cancer, soft tissue tumor, breast cancer, chronic myelogenous luekemia, esophageal cancer and gastric cancer as well as methods for screening for useful substances for either or both of treating and preventing cancer.

In particular, the present invention arises from the discovery that SUV420H1 or SUV420H2 gene is overexpressed in cancer cells and double-stranded molecules composed of a sense strand and an antisense strand, wherein the sense strand includes a nucleotide sequence corresponding to a target sequence selected from the group consisting of SEQ ID NOs: 29, 30, 31 and 32 and the antisense strand includes a sequence which is complementary to the sense strand, wherein the sense and the antisense strands of the molecule hybridize to each other to form a double-stranded molecule, which inhibit SUV420H1 or SUV420H2 expression, are effective for inhibiting cellular growth of cancer cells.

Therefore, in one aspect, the present invention provides isolated double-stranded molecules, that when introduced into a cell expressing either or both of an SUV420H1 and SUV420H2 gene, inhibit the expression of an SUV420H1 or SUV420H2 gene as well as cell proliferation, the molecule including a sense strand and an antisense strand complementary thereto, and the strands hybridized to each other to form the double-stranded molecule. These double-stranded molecules may be utilized in an isolated state or encoded in vectors and expressed from the vectors. Accordingly, in another aspect, the present invention provides vectors encoding the double-stranded molecules and host cells carrying the vectors.

In another aspect, the present invention provides methods for inhibiting cancer cell growth or treating cancer, particularly cancers including bladder cancer, cervical cancer, osteosarcoma, lung cancer, soft tissue tumor, breast cancer, chronic myelogenous luekemia, esophageal cancer and gastric cancer, by administering the double-stranded molecules or vectors of the present invention to a subject in need thereof. Such methods encompass administering to a subject a composition containing one or more of the double-stranded molecules or vectors of the present invention.

In another aspect, the present invention provides compositions for treating cancers, including bladder cancer, cervical cancer, osteosarcoma, lung cancer, soft tissue tumor, breast cancer, chronic myelogenous luekemia, esophageal cancer and gastric cancer, such compositions containing at least one of the double-stranded molecules or vectors of the present invention.

In yet another aspect, the present invention provides methods of detecting or diagnosing cancer in a subject by determining an expression level of SUV420H1 or SUV420H2 in a subject-derived biological sample. An increase in the expression level of the gene as compared to a normal control level of the gene indicates the presence of cancer in the subject or that the subject suffers from cancer, particularly cancers including bladder cancer, cervical cancer, osteosarcoma, lung cancer, soft tissue tumor, breast cancer, chronic myelogenous luekemia, esophageal cancer and gastric cancer.

The present invention also relates to the discovery that an expression level of SUV420H2 gene correlates to poor prognosis of cancer. Therefore, in another aspect, present invention provides methods for assessing or determining the prognosis of a subject with cancer, such methods including the steps of determining the expression level of the SUV420H2 gene, comparing it to a pre-determined reference expression level and assessing or determining the prognosis of the subject based on the comparison.

In a further aspect, the present invention provides methods of screening for candidate substances for either or both of treating and preventing cancer. Such substances bind with SUV420H1 or SUV420H2 polypeptide, reduce the biological activity of the SUV420H1 or SUV420H2 polypeptide, reduce the expression level of SUV420H1 or SUV420H2 gene, or reduce the expression or activity of a reporter gene serving as a surrogate for the SUV420H1 or SUV420H2 gene.

In a further aspect, the present invention provides a kit for detecting or diagnosing cancer, or assessing or determining the prognosis of cancer, which comprises a reagent for detecting an mRNA, protein or biological activity of SUV420H1 or SUV420H2.

More specifically, the present invention provides the following [1] to [28]:

[1] A method of detecting or diagnosing cancer in a subject, comprising determining an expression level of an SUV420H1 or SUV420H2 gene in a subject-derived biological sample, wherein an increase of said level compared to a normal control level of said gene indicates the presence of cancer in said subject, or that said subject suffers from cancer, wherein the expression level is determined by any one of a method selected from the group consisting of:

(a) detecting an mRNA of an SUV420H1 or SUV420H2 gene;

(b) detecting a protein encoded by an SUV420H1 or SUV420H2 gene; and

(c) detecting a biological activity of a protein encoded by an SUV420H1 or SUV420H2 gene;

[2] The method of [1], wherein said increase is at least 10% greater than said normal control level;

[3] The method of [1], wherein the subject-derived biological sample comprises a biopsy specimen, sputum, blood, pleural effusion or urine;

[4] A kit for detecting or diagnosing cancer, which comprises a reagent selected from the group consisting of:

(a) a reagent for detecting an mRNA of an SUV420H1 or SUV420H2 gene;
(b) a reagent for detecting a protein encoded by an SUV420H1 or SUV420H2 gene; and
(c) a reagent for detecting a biological activity of a protein encoded by an SUV420H1 or SUV420H2 gene;

[5] The kit of [4], wherein the reagent is a probe or primer set that bind to the mRNA of the SUV420H1 or SUV420H2 gene;

[6] The kit of [4], wherein the reagent is an antibody against the protein encoded by the SUV420H1 or SUV420H2 gene, or a fragment thereof;

[7] A method of screening for a candidate substance for treating or preventing cancer, or inhibiting cancer cell growth, said method comprising the steps of:

(a) contacting a test substance with an SUV420H1 or SUV420H2 polypeptide;
(b) detecting the binding activity between the polypeptide and the test substance; and
(c) selecting the test substance that binds to the polypeptide;

[8] A method of screening for a candidate substance for treating or preventing cancer or inhibiting cancer cell growth, said method comprising the steps of:

(a) contacting a test substance with a cell expressing an SUV420H1 or SUV420H2 gene;
(b) detecting the expression level of the SUV420H1 or SUV420H2 gene in the cell; and
(c) selecting the test substance that reduces the expression level of the SUV420H1 or SUV420H2 gene in comparison with the expression level in the absence of the test substance;

[9] A method of screening for a candidate substance for treating or preventing cancer or inhibiting cancer cell growth, said method comprising the steps of:

(a) contacting a test substance with an SUV420H1 or SUV420H2 polypeptide;
(b) detecting a biological activity of the polypeptide of step (a); and
(c) selecting the test substance that suppresses the biological activity of the polypeptide in comparison with the biological activity detected in the absence of the test substance;

[10] The method of [9], wherein the biological activity is cell proliferation enhancing activity or methyltransferase activity or anti-apoptotic activity;

[11] A method of screening for a candidate substance for treating or preventing cancer or inhibiting cancer cell growth, said method comprising the steps of:

(a) contacting a test substance with a cell into which a vector comprising the transcriptional regulatory region of an SUV420H1 or SUV420H2 gene and a reporter gene that is expressed under the control of the transcriptional regulatory region has been introduced,
(b) measuring the expression or activity of said reporter gene; and
(c) selecting the test substance that reduces the expression or activity level of said reporter gene, in comparison with the level in the absence of the test substance;

[12] An isolated double-stranded molecule that, when introduced into a cell, inhibits the expression of an SUV420H1 or SUV420H2 gene as well as cell proliferation, the molecule comprising a sense strand and an antisense strand complementary thereto, the strands hybridized to each other to form the double-stranded molecule, the sense strand comprising the nucleotide sequence corresponding to a target sequence selected from the group consisting of SEQ ID NOs:29, 30, 31 and 32;

[13] The double-stranded molecule of [12], wherein the sense strand hybridizes with antisense strand at the target sequence to form the double-stranded molecule having between 19 and 25 nucleotide pairs in length;

[14] The double-stranded molecule of [12], which consists of a single polynucleotide comprising both the sense and antisense strands linked by an intervening single-strand;

[15] The double-stranded molecule of [14], which has the general formula 5′-[A]-[B]-[A′]-3′ or 5′-[A′]-[B]-[A]-3′, wherein [A] is the sense strand comprising a nucleotide sequence corresponding to a target sequence selected from the group consisting of SEQ ID NOs: 29, 30, 31 and 32, [B] is an intervening single-strand consisting of 3 to 23 nucleotides, and [A′] is an antisense strand comprising a complementary sequence to [A];

[16] A vector encoding the double-stranded molecule of any one of [12] to [15];

[17] Vectors comprising each of a combination of a polynucleotide comprising a sense strand nucleic acid and an antisense strand nucleic acid, wherein said sense strand nucleic acid comprises a nucleotide sequence corresponding to SEQ ID NO: 29, 30, 31 or 32 and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing SUV420H1 or SUV420H2 gene, inhibit the cell proliferation;

[18] A method of either or both of treating and preventing cancer, or inhibiting cancer cell growth in a subject, comprising administering to the subject a pharmaceutically effective amount of a double-stranded molecule against an SUV420H1 or SUV420H2 gene or a vector encoding the double-stranded molecule, wherein the double-stranded molecule, when introduced into a cell, inhibits the expression of an SUV420H1 or SUV420H2 gene as well as cell proliferation, the molecule comprising a sense strand and an antisense strand complementary thereto, the strands hybridized to each other to form the double-stranded molecule;

[19] The method of [18], wherein the double-stranded molecule is that of any one of [12] to [15];

[20] A composition for either or both of treating and preventing cancer, or inhibiting cancer cell growth, which comprises a pharmaceutically effective amount of a double-stranded molecule against an SUV420H1 or SUV420H2 gene or a vector encoding the double-stranded molecule, wherein the double-stranded molecule, when introduced into a cell, inhibits expression of an SUV420H1 or SUV420H2 gene as well as cell proliferation, the molecule comprising a sense strand and an antisense strand complementary thereto, the strands hybridized to each other to form the double-stranded molecule, and a pharmaceutically acceptable carrier;

[21] The composition of [20], wherein the double-stranded molecule is that of any one of [12] to [15];

22. A method for monitoring, assessing or determining the prognosis of a subject with cancer, which method comprises the steps of:

(a) detecting an expression level of an SUV420H2 gene in a subject-derived biological sample;
(b) comparing the expression level detected in step (a) to a control level; and
(c) assessing or determining the prognosis of the patient based on the comparison of step (b);

[23] The method of [22], wherein the control level is a good prognosis control level and an increase of the expression level as compared to the control level is correlated with poor prognosis.

[24] The method of [22], wherein the expression level is determined by a method selected from the group consisting of:

(a) detecting an mRNA of an SUV420H2 gene;
(b) detecting a protein encoded by an SUV420H2 gene; and
(c) detecting a biological activity of a protein encoded by an SUV420H2 gene;

[25] The method of [22], wherein the subject-derived biological sample comprises a biopsy specimen;

[26] A kit for monitoring, assessing or determining the prognosis of a subject with cancer, which comprises a reagent selected from the group consisting of:

(a) a reagent for detecting an mRNA of an SUV420H2 gene;
(b) a reagent for detecting a protein encoded by an SUV420H2 gene; and
(c) a reagent for detecting a biological activity of a protein encoded by an SUV420H2 gene;

[27] The kit of [26], wherein the reagent is a probe or primer set that bind to the mRNA of the SUV420H2 gene; and

[28] The kit of [26], wherein the reagent is an antibody against the protein encoded by the SUV420H2 gene, or a fragment thereof.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention.

These and other objects and features of the invention will become more fully apparent when the following detailed description is read in conjunction with the accompanying figures and examples. However, it will also be understood that both the foregoing summary of the present invention and the following detailed description are of a exemplified embodiments, and not restrictive of the present invention or other alternate embodiments of the present invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments that follows:

FIG. 1 depicts the elevated SUV420H1/H2 expressions in bladder cancer in British and Japanese patients. A: SUV420H1 and SUV420H2 gene expressions in normal and tumor bladder tissues in British cases. Expression levels of SUV420H1/H2 were analyzed by quantitative real-time PCR, and the result is shown by box-whisker plot (median 50% boxed). Relative mRNA expression shows the value normalized by GAPDH and SDH expressions. Mann-Whitney U-test was used for statistical analysis. B: Comparison of SUV420H1/H2 expression between normal and tumor bladder tissues in Japanese patients. Signal intensity of each sample was analyzed by cDNA microarray, and the result is shown by box-whisker plot (median 50% boxed). Mann-Whitney U-test was used for statistical analysis.

FIG. 1C C: Expression levels of SUV420H2 in 16 normal tissues and bladder tumor tissues. Data were normalized by GAPDH and SDH expressions, and relative SUV420H2 expression shows the ratio compared to the value in normal bladder tissue (1=SAEC expression).

FIG. 2 depicts the elevated SUV420H1/H2 expressions in various types of human cancer. A: comparison of SUV420H1 expression between normal and tumor tissues in cervical cancer, osteosarcoma, lung cancer (SCLC) and soft tissue tumor. Signal intensity of each sample was analyzed by cDNA microarray, and the result is shown by box-whisker plot (median 50% boxed). Mann-Whitney U test was used for the statistical analysis.

FIG. 2B B: Comparison of SUV420H2 expression between normal and tumor tissues in breast cancer, chronic myelogenous leukemia (CML), esophageal, gastric cancer and lung cancer (SCLC and NSCLC). Signal intensity of each sample was analyzed by cDNA microarray, and the result is shown by box-whisker plot (median 50% boxed). Mann-Whitney U test was used for the statistical analysis.

FIG. 2C-D C: Representative cases for strong positive, weakly positive and negative SUV420H2 staining in lung cancer and normal lung tissues on the tissue microarray. D: Kaplan-Meier estimates of overall survival time of patients with NSCLC (P=0.0023, log-rank test).

FIG. 3 depicts the expression of SUV420H1 and SUV420H2 in normal and tumor bladder and lung cancer cell lines, and the suppression of endogenous expression of SUV420H1 and SUV420H by two SUV420H1-specific siRNAs. A,B: Expression of SUV420H1(A) and SUV420H2(B) in 3 normal cell lines, 14 bladder cancer cell lines and 5 lung cancer cell lines. Expression levels were analyzed by quantitative real-time PCR, and relative mRNA expression shows the value normalized by GAPDH and SDH expressions.

FIG. 3C-D C,D: Quantitative real-time PCR showing suppression of endogenous expression of SUV420H1 (C) and SUV420H2 (D) by two SUV420H1-specific siRNAs (siSUV420H1#1 and #2) and two SUV420H2-specific siRNAs (siSUV420H2#1 and #2) in A549 and SBC5 cells. siEGFP was used as a control. mRNA expression levels were normalized by GAPDH and SDH expressions, and values are relative to siEGFP (siEGFP=1).

FIG. 4 depicts the effects of SUV420H1 and SUV420H2 siRNA knockdown on the viability of bladder cancer cell lines and lung cancer cell lines and on cell cycle kinetics. A,B: Effects of SUV420H1 (A) and SUV420H2 (B) siRNA knockdown on the viability of two bladder cancer cell lines (SW780 and RT4) and three lung cancer cell lines (A549, LC319 and SBC5). Relative cell number shows the value normalized to siEGFP-treated cells. Results are the mean+/−SD in three independent experiments. P-values were calculated using Student's t-test (*, P<0.05; **, P<0.01; ***, P<0.001).

FIG. 4C-D C: Effect of siSUV420H2 on cell cycle kinetics in A549 cells. Cell cycle distribution was analyzed by flow cytometry after staining with propidium iodide as described in Materials and Methods. D: Numerical analysis of the FACS result, classifying cells by cell cycle status. The proportion of T-REx-SUV420H2 cells in S and G₂/M phases is slightly higher than control cells (T-REx-Mock and T-REx-CAT). Mean+/−SD of three independent experiments. Fisher's PLSD Post-Hoc test was used to calculate P values (*, P<0.05; **, P<0.01).

FIG. 4E-F E: Colony formation assay of SW780, A549 and SBC5 cells 72 hours after siSUV420H2 treatment. F: Effect of siSUV420H2 on cell cycle kinetics in SBC5 cell. Cell cycle distribution was analyzed by flow cytometry after coupled staining with fluorescein isothiocyanate (FITC)-conjugated anti-BrdU and 7-amino-actinomycin D (7-AAD).

FIG. 5 depicts the effect of SUV420H2 knockdown-dependent apoptosis induction of cancer cells. A: Western blotting using anti-PARP1 and anti-Cleaved Caspase-3 antibodies. Anti-GAPDH antibody was used as an internal control. B: TUNEL assay using Fluorescent system. Apoptotic cells were stained by Alexa Fluor (registered trademark) 488 and nuclei were counterstained by Propidium Indide; Alexa Fluor™594.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, GenBank Accession or other sequence, patent or patent application mentioned in this specification is specifically incorporated by reference herein in its entirety. However, nothing herein is to be construed as an admission that the invention is not entitled to antedate such disclosure by virtue of prior invention.

Unless otherwise defined, all technical and scientific terms used herein have the same meaning as commonly understood by one of ordinary skill in the art to which the present invention belongs. In case of conflict, the present specification, including definitions, will control. In addition, the materials, methods, and examples are illustrative only and not intended to be limiting.

### DEFINITION

The words “a”, “an”, and “the” as used herein mean “at least one” unless otherwise specifically indicated.

The terms “isolated” and “purified” used in relation with a substance (e.g., polypeptide, antibody, polynucleotide, etc.) indicates that the substance is substantially free from at least one substance that can also be included in the natural source. Thus, an isolated or purified antibody refers to antibodies that are substantially free of cellular material for example, carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced.

Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, in some embodiments it is also substantially free of culture medium, which includes preparations of polypeptide with culture medium less than about 20%, 10%, or 5% of the volume of the protein preparation. When the polypeptide is produced by chemical synthesis, in some embodiments it is substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In one embodiment, proteins including antibodies of the present invention are isolated or purified.

In the context of the present invention, the phrase “SUV420H1 gene” or “SUV420H2 gene” encompass polynucleotides that encode the human SUV420H1 or SUV420H2 gene or any of the functional equivalents of the human SUV420H1 or SUV420H2 gene. The SUV420H1 or SUV420H2 gene can be obtained from nature as naturally occurring polynucleotides via conventional cloning methods or through chemical synthesis based on the selected nucleotide sequence. Methods for cloning genes using cDNA libraries and such are well known in the art.

To the extent that the methods and compositions of the present invention find utility in the context of “prevention” and “prophylaxis”, such terms are interchangeably used herein to refer to any activity that reduces the burden of mortality or morbidity from disease. Prevention and prophylaxis can occur “at primary, secondary and tertiary prevention levels”. While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

To the extent that certain embodiments of the present invention encompass the treatment and/or prophylaxis of cancer and/or the prevention of postoperative recurrence, such methods may include any of the following steps: the surgical removal of cancer cells, the inhibition of the growth of cancerous cells, the involution or regression of a tumor, the induction of remission and suppression of occurrence of cancer, the tumor regression, and the reduction or inhibition of metastasis. Effective treatment and/or the prophylaxis of cancer decreases mortality and improves the prognosis of individuals having cancer, decreases the levels of tumor markers in the blood, and alleviates detectable symptoms accompanying cancer. A treatment may also deemed “efficacious” if it leads to clinical benefit such as, reduction in expression of the SUV420H1 or SUV420H2 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, for example, an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those modified after translation in cells (e.g., hydroxyyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine sulfoxide, methionine methyl sulfonium). The phrase “amino acid mimetic” refers to chemical compounds that have different structures but similar functions to general amino acids.

Amino acids can be referred to herein by their commonly known three letter symbols or the one-letter symbols recommended by the IUPAC-IUB Biochemical Nomenclature Commission.
The terms “gene”, “polynucleotide”, “oligonucleotide”, “nucleic acid”, and “nucleic acid molecule” are used interchangeably unless otherwise specifically indicated and are similarly to the amino acids referred to by their commonly accepted single-letter codes.
The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, for example, a nutrient broth or gel in which an organism has been propagated, which contains cellular components, for example, proteins or polynucleotides.

Unless otherwise defined, the terms “cancer” refers to cancers over-expressing the SUV420H1gene or SUV420H2 gene, such as bladder cancer, cervical cancer, osteosarcoma, lung cancer, soft tissue tumor, breast cancer, chronic myelogenous leukemia (CML), esophageal cancer and gastric cancer.

Genes and Proteins:

The SUV420H1 and SUV420H2 are histone methyltransferases that catalyze di- and trimethylation of histone H4K20 which is a characteristic of pericentric heterochromatin. According to current models, H3K9me3 which are a result of SUV39H activity stabilizes heterochromatin protein 1 (HP1) binding at heterochromatin, and HP1 proteins then recruit the histone methyltransferases SUV420H1 and SUV420H2 which in turn, trimethylate H4K20. SUV420H1 and SUV420H2 are alternatively spliced transcript variants.

The nucleic acid sequences of the above mentioned genes and the amino acid sequences of the polypeptides encoded by them are known in the art. For example, the exemplary amino acid sequences of SUV420H1 and SUV420H2 polypeptide include, but are not limited to, the amino acid sequences shown in SEQ ID NOs: 24 and 26, for SUV420H1, and SEQ ID No:28 for SUV420H2 and they are also available via GenBank accession numbers, NP_{—}060105.3 and NP_{—}057112.3 for SUV420H1, and NP_{—}116090.2 for SUV420H2, respectively. Thus, the exemplary nucleic acid sequences of the SUV420H1gene and the SUV420H2 gene may contain nucleic acid sequences encoding amino acid sequences shown in SEQ ID NOs: 24 and 26 for SUV420H1, and SEQ ID NO: 28 for SUV420H2, respectively. The examples of such nucleic acid sequences include nucleic acid sequences shown in SEQ ID NOs: 23 and 25 for SUV420H1, and SEQ ID NO: 27 for SUV420H2, but are not limited to. These nucleic acid sequence data are also available via GenBank accession numbers, NM_{—}017635.3 and NM_{—}016028.4 for SUV420H1, and NM_{—}032701.3 for SUV420H2, respectively.

According to one aspect of the present invention, functional equivalents of a polypeptide are also considered to be the “polypeptide”. Herein, a “functional equivalent” of a polypeptide is a polypeptide that has a biological activity equivalent to the polypeptide. Namely, any polypeptide that retains a biological ability of the polypeptide may be used as such a functional equivalent in the present invention. Such functional equivalents include those wherein one or more amino acids are substituted, deleted, added, or inserted to the natural occurring amino acid sequence of the polypeptide. Alternatively, functional equivalents may be composed of an amino acid sequence having at least about 80% homology (also referred to as sequence identity) to the amino acid sequence of the polypeptide, at least about 90% to 95% homology, or about 96%, 97%, 98% or 99% homology. The homology of a particular polynucleotide or polypeptide can be determined by following the algorithm in “Wilbur and Lipman, Proc Natl Acad Sci USA 80: 726-30 (1983)”. In other embodiments, a functional equivalent may be a polypeptide encoded by a polynucleotide that hybridizes to the polynucleotide having the natural occurring nucleotide sequence of the gene under a stringent condition.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a functional equivalent to that of the polypeptide, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times the level of background, or 10 times the level of background. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a hybridization condition for isolating a polynucleotide encoding a functional equivalent of a polypeptide can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and incubating at 68 degrees C. for 1 hour or longer. The following washing step can be conducted, for example, in a low stringent condition. An exemplary low stringent condition may include 42 degrees C., 2×SSC, 0.1% SDS, and 50 degrees C., 2×SSC, 0.1% SDS. High stringency conditions may also be used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acids in a polypeptide do not influence the function of the polypeptide. In fact, mutated or modified polypeptides, having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a polypeptide results in a polypeptide with similar functions, are acceptable in the context of the instant invention. Thus, the polypeptides of the present invention may have an amino acid sequence wherein one, two or even more amino acids are added, inserted, deleted, and/or substituted in an originally disclosed reference sequence.

So long as the biological activity of the polypeptide is maintained, the number of amino acid mutations is not particularly limited. However, it is typical to alter 5% or less of the amino acid sequence. Accordingly, in one embodiment, the number of amino acids to be mutated in such a mutant is generally 30 amino acids or less, 20 amino acids or less, 10 amino acids or less, 5 or 6 amino acids or less, or 3 or 4 amino acids or less.

An amino acid residue to be mutated may be mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified polypeptides are included in functional equivalents of a polypepitde. However, the present invention is not restricted thereto and functional equivalents of a polypeptide may include non-conservative modifications, so long as at least one biological activity of the polypeptide is retained. Furthermore, the modified polypeptides do not exclude polymorphic variants, interspecies homologues, and those encoded by alleles of these polypeptides.

An example of a polypeptide modified by addition of one or more amino acid residues is a fusion protein of the SUV420H1 or SUV420H2 polypeptide. Fusion proteins can be made by techniques well known to a person skilled in the art, for example, by linking the DNA encoding the SUV420H1 or SUV420H2 gene with a DNA encoding another peptide or protein, so that the frames match, inserting the fusion DNA into an expression vector and expressing it in a host. The “other” component of the fusion protein is typically a small epitope composed of several to a dozen amino acids. There is no restriction as to the peptides or proteins fused to the SUV420H1 or SUV420H2 polypeptide so long as the resulting fusion protein retains any one of the objective biological activities of the SUV420H1 or SUV420H2 polypeptide. Exemplary fusion proteins contemplated by the instant invention include fusions of the SUV420H1 or SUV420H2 polypeptide and other small peptides or proteins such as FLAG (Hopp T P, et al., Biotechnology 6: 1204-10 (1988)), a polyhistidine (His-tag) such as 6×His containing six His (histidine) residues or 10×His containing 10 His residues, Influenza aggregate or agglutinin (HA), human c-myc fragment, Vesicular stomatitis virus glycoprotein (VSV-GP), p18HIV fragment, T7 gene 10 protein (T7-tag), human simple herpes virus glycoprotein (HSV-tag), E-tag (an epitope on monoclonal phage), SV40T antigen fragment, lck tag, alpha-tubulin fragment, B-tag, Protein C fragment, and the like. Other examples of proteins that can be fused to a protein of the invention include GST (glutathione-S-transferase), Influenza agglutinin (HA), immunoglobulin constant region, beta-galactosidase, MBP (maltose-binding protein), and such.

Other examples of modified proteins contemplated by the present invention include polymorphic variants, interspecies homologues, and those encoded by alleles of these proteins.

Also, in the context of the present invention, a gene encompasses polynucleotides that encode such functional equivalents of the polypeptide. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

Double-Stranded Molecule:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g., double-stranded chimera of DNA and RNA (dsD/R-NA) or small hairpin chimera of DNA and RNA (shD/R-NA)). Herein, “double-stranded molecule” is also referred to as “double-stranded nucleic acid “,” double-stranded nucleic acid molecule”, “double-stranded polynucleotide”, “double-stranded polynucleotide molecule”, “double-stranded oligonucleotide” and “double-stranded oligonucleotide molecule”.

As used herein, the term “target sequence” refers to a nucleotide sequence within the mRNA or cDNA sequence of a target gene, which will result in suppress of translation of the whole mRNA of the target gene if a double-stranded nucleic acid molecule containing the sequence is introduced into a cell expressing the gene. A nucleotide sequence within the mRNA or cDNA sequence of a target gene can be determined to be a target sequence when a double-stranded molecule comprising a sequence corresponding to the target sequence inhibits expression of the gene in a cell expressing the gene. When a target sequence is shown by cDNA sequence, a sense strand sequence of a double-stranded cDNA, i.e., a sequence that mRNA sequence is converted into DNA sequence, is used for defining a target sequence. A double-stranded molecule is composed of a sense strand that has a sequence corresponding to a target sequence and an antisense strand that has a complementary sequence to the target sequence, and the antisense strand hybridizes with the sense strand at the complementary sequence to form a double-stranded molecule. Herein, the phrase “corresponding to” means converting a target sequence according to the kind of nucleotides that constitute a sense strand of a double-stranded molecule. For example, when a target sequence is shown in a DNA sequence and a sense strand of a double-stranded molecule has an RNA region, base “t”s within the RNA region are replaced with base “u”s. On the other hand, when a target sequence is shown in an RNA sequence and a sense strand of a double-stranded molecule has a DNA region, base “u”s within the DNA region are replaced with “t”s. For example, when a target sequence is the RNA sequence shown in SEQ ID NO: 29, 30, 31 or 32 and the 3′ side half region of the sense strand of the double-stranded molecule is composed of DNA, “a sequence corresponding to a target sequence” is

(for SEQ ID NO: 29)
″5′-GAGUUCUGCGAGTGTTACA-3′′′,
(for SEQ ID NO: 30)
″5′-GAAAUUAUUCAAAGAACAT-3′′′,
(for SEQ ID NO: 31)
″5′-GGAUCUGAGCCCTGACCCT-3′′′
or
(for SEQ ID NO: 32)
″5′-GCAUAGCUCUGACCCTGGA-3′′′.

Also, a complementary sequence to a target sequence for an antisense strand of a double-stranded molecule can be defined according to the kind of nucleotides that constitute the antisense strand. For example, when a target sequence is the RNA sequence shown in SEQ ID NO: 29, 30, 31 or 32 and the 5′ side region of the antisense strand of the double-stranded molecule is composed of DNA, “a complementary sequence to a target sequence is

(for SEQ ID NO: 29)
″5′-TGTAACACUCGCAGAACUC-3′′′,
(for SEQ ID NO: 30)
″5′-ATGTTCUUU-GAAUAAUUUC-3′′′,
(for SEQ ID NO:31)
″5′-AGGGTCAGGGCUCAGAUCC-3′′′
or
(for SEQ ID NO: 32)
″5′-TCCAGGGTCAGAGCUAUGC-3′′′.

On the other hand, for example, when a double-stranded molecule is composed of RNA, the sequence corresponding to a target sequence shown in SEQ ID NO: 29, 30, 31 or 32 is the ribonucleotide sequence shown in SEQ ID NO: 29, 30, 31 or 32 and the complementary sequence to the target sequence is the ribonucleotide sequence shown in SEQ ID NO: 29, 30, 31 or 32.

A double-stranded molecule may have one or two 3′ overhang(s) having 2 to 5 nucleotides in length (e.g., uu) and/or a loop sequence that links a sense strand and an antisense strand to form hairpin structure, in addition to a sequence corresponding to a target sequence and complementary sequence thereto.

As used herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a part of sense nucleic acid sequence of the target gene (also referred to as “sense strand”), a part of antisense nucleic acid sequence of the target gene (also referred to as “antisense strand”) or both (nucleotide “t” is replaced with “u” in an siRNA). The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another that have annealed together via the complementary sequences to form a double-stranded RNA molecule. The nucleotide sequence of the two strands may include not only the “sense” or “antisense” RNAs selected from a protein coding sequence of target gene sequence, but also a nucleotide sequence selected from non-coding region of the target gene.

The term “shRNA”, as used herein, refers to an siRNA having a stem-loop structure, composed of the first and second regions complementary to one another, i.e., sense and antisense strands. The degree of complementarity and orientation of the regions is sufficient such that base pairing occurs between the regions, the first and second regions is joined by a loop region, and the loop results from a lack of base pairing between nucleotides (or nucleotide analogs) within the loop region. The loop region of an shRNA is a single-stranded region intervening between the sense and antisense strands and may also be referred to as an “intervening single-strand”.

As used herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double-stranded molecule may contain RNA and DNA. Standard techniques of introducing siD/R-NA into the cell are used. The siD/R-NA includes a part of sense nucleic acid sequence of the target gene (also referred to as “sense strand”), a part of antisense nucleic acid sequence of the target gene (also referred to as “antisense strand”) or both (nucleotide “t” is replaced with “u” in RNA). The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of the first and second regions complementary to one another, i.e., sense and antisense strands. The degree of complementarity and orientation of the regions is sufficient such that base pairing occurs between the regions, the first and second regions are joined by a loop region, and the loop results from a lack of base pairing between nucleotides (or nucleotide analogs) within the loop region. The loop region of an shD/R-NA is a single-stranded region intervening between the sense and antisense strands and may also be referred to as an “intervening single-strand”.

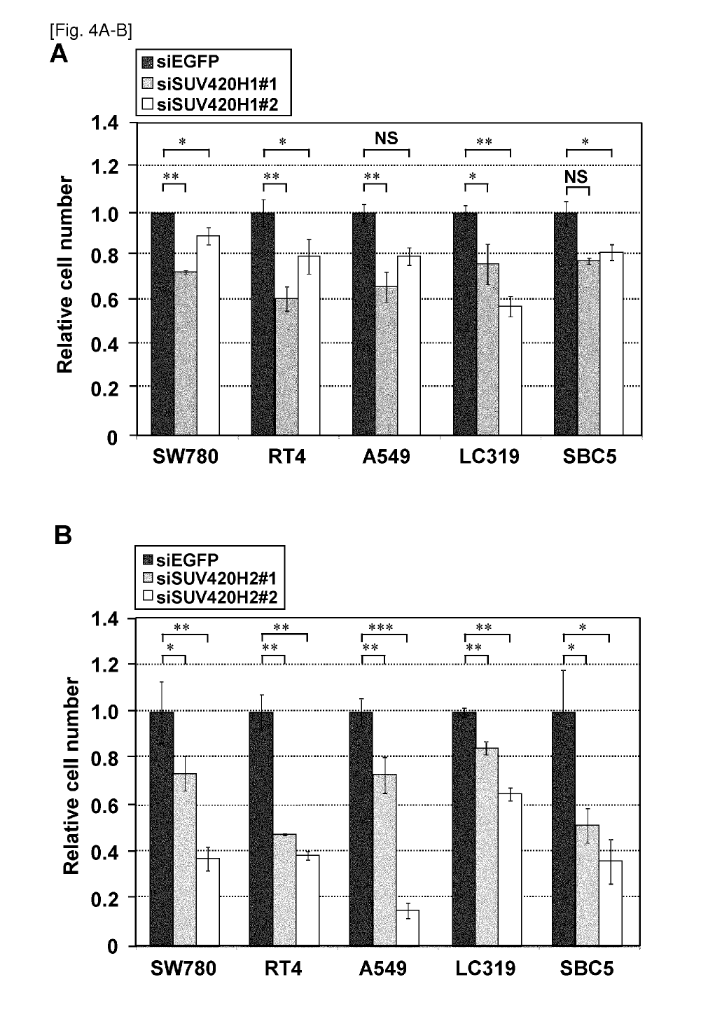

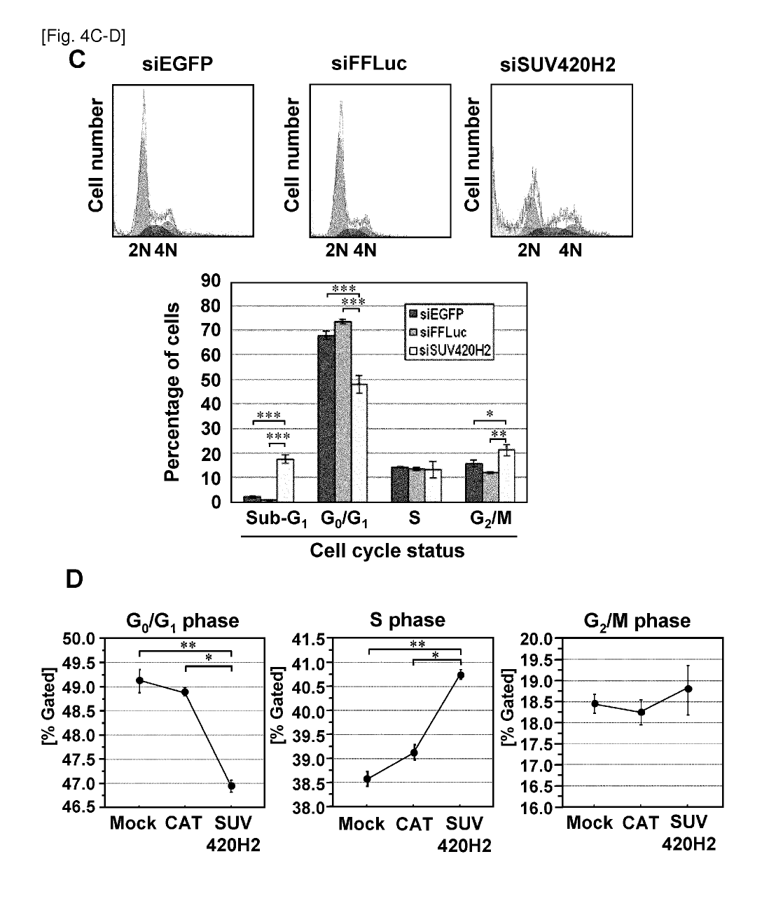

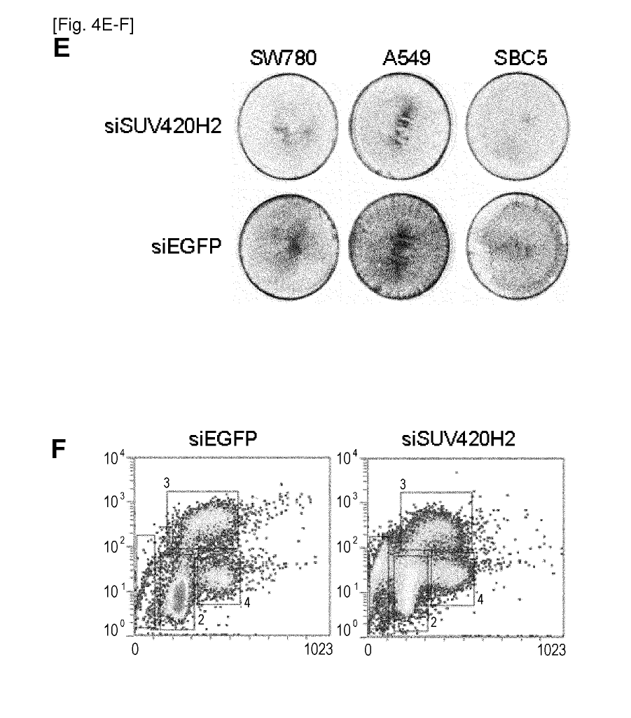

In one embodiment, the present invention provides a double-stranded molecule against SUV420H1 or SUV420H2, of which the antisense strand hybridizes to the SUV420H1 or SUV420H2 mRNA, induces degradation of the SUV420H1 or SUV420H2 mRNA by associating with the normally single-stranded mRNA transcript of the gene, thereby interfering with translation and thus, inhibiting expression of the protein. As demonstrated herein, the expression of SUV420H1 or SUV420H2 in cancer cell lines, which overexpress SUV420H1 or SUV420H2 gene, is inhibited by dsRNAs against SUV420H1 or SUV420H2 gene, and consequently, the growth of those cancer cell lines is suppressed (FIGS. 4A and 4B). Therefore, the present invention provides isolated double-stranded molecules that are capable of inhibiting the expression of the SUV420H1 or SUV420H2 gene as well as cell growth when introduced into a cell expressing the gene. The double-stranded molecules of the present invention are useful for inhibiting cancer cell growth relating to the overexpression of the SUV420H1 or SUV420H2 gene, therefore, they may provide new methods for treating cancers. For example, the double-stranded molecules of the present invention are suitable for treating cancers such as bladder cancer, cervical cancer, osteosarcoma, lung cancer (e.g. SCLC or NSCLC), soft tissue tumor, breast cancer, chronic myelogenous leukemia, esophageal cancer and gastric cancer, which overexpress either or both of SUV420H1 and SUV420H2 gene.

The target sequences of the double-stranded molecule against SUV420H1 or SUV420H2 gene include, for example, nucleotide sequences selected from among SEQ ID NOs: 29, 30, 31 and 32.

The target sequences for SUV420H1 include, for example,

(SEQ ID NO: 29)
5′-GAGUUCUGCGAGUGUUACA-3′
or
(SEQ ID NO: 30)
5′-GAAAUUAUUCAAAGAACAU-3′.

Also, the target sequences for SUV420H2 include, for example,

(SEQ ID NO: 31)
5′-GGAUCUGAGCCCUGACCCU-3′
or
(SEQ ID NO: 32)
5′-GCAUAGCUCUGACCCUGGA-3′.

Specifically, the present invention provides the following double-stranded molecules [1] to [18]:

[1] An isolated double-stranded molecule that, when introduced into a cell expressing either or both of an SUV420H1 and SUV420H2 gene, inhibits the expression of the SUV420H1 or SUV420H2 gene and cell proliferation, wherein the double-stranded molecule contains a sense strand and an antisense strand complementary thereto, hybridized to each other to form the double-stranded molecule, wherein the sense strand contains a nucleotide sequence corresponding to a part of SUV420H1 or SUV420H2 gene sequence;

[2] The double-stranded molecule of [1], wherein the double-stranded molecule acts on the mRNA of the SUV420H1 or SUV420H2 gene, matching a target sequence selected from among SEQ ID NOs:29, 30, 31 and 32;

[3] The double-stranded molecule of [1] or [2], wherein the sense strand contains a nucleotide sequence corresponding to a target sequence selected from among SEQ ID NOs: 29, 30, 31 and 32;

[4] The double-stranded molecule of any one of [1] to [3], wherein the sense strand hybridizes with the antisense strand at the target sequence to form the double-stranded molecule having a length of less than about 100 nucleotide pairs in length;

[5] The double-stranded molecule of any one of [1] to [4], wherein the sense strand hybridizes with the antisense strand at the target sequence to form the double-stranded molecule having a length of less than about 75 nucleotide pairs in length;

[6] The double-stranded molecule of [5], wherein the sense strand hybridizes with the antisense strand at the target sequence to form the double-stranded molecule having a length of less than about 50 nucleotide pairs in length;

[7] The double-stranded molecule of [6] wherein the sense strand hybridizes with the antisense strand at the target sequence to form the double-stranded molecule having a length of less than about 25 nucleotide pairs in length;

[8] The double-stranded molecule of [7], wherein the sense strand hybridizes with the antisense strand at the target sequence to form the double-stranded molecule having a length of between about 19 and about 25 nucleotide pairs in length;

[9] The double-stranded molecule of any one of [1] to [8], composed of a single polynucleotide having both the sense and antisense strands linked by an intervening single-strand;

[10] The double-stranded molecule of [9], having the general formula 5′-[A]-[B]-[A′]-3′ or 5′-[A′]-[B]-[A]-3′, wherein [A] is the sense strand containing a nucleotide sequence corresponding to a target sequence selected from among SEQ ID NOs:29, 30, 31 and 32, [B] is the intervening single-strand composed of 3 to 23 nucleotides, and [A′] is the antisense strand containing a sequence complementary to [A];

[11] The double-stranded molecule of any one of [1] to [10], composed of RNA;

[12] The double-stranded molecule of any one of [1] to [10], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking the 3′-end of the antisense strand, or both of a region flanking the 5′-end of the sense strand and a region flanking the 3′-end of the antisense strand are RNA;

[17] The double-stranded molecule of [16], wherein the flanking region is composed of 9 to 13 nucleotides; and

[18] The double-stranded molecule of any one of [1] to [17], wherein the molecule contains one or two 3′ overhang(s).

The double-stranded molecule of the present invention will be described in more detail below.

Methods for designing double-stranded molecules having the ability to inhibit target gene expression in cells are known (See, for example, U.S. Pat. No. 6,506,559, herein incorporated by reference in its entirety). For example, a computer program for designing siRNAs is available from the Ambion website (www.ambion.com/techlib/misc/siRNA_finder.html).

The computer program selects target nucleotide sequences for double-stranded molecules based on the following protocol.

Selection Of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Any other algorithms developed for designing siRNA may be also used for designing target sequences of the double-stranded molecules of the present invention.

In the present invention, nucleotide sequences shown in SEQ ID NOs:29, 30, 31 and 32. are demonstrated to be suitable for target sequences of the double-stranded molecules of the present invention.

Double-stranded molecules targeting the above-mentioned target sequences were respectively examined and it was confirmed that they possessed ability to suppress the growth of cells expressing the SUV420H1 and SUV420H2 gene. Therefore, one embodiment of the present invention provides double-stranded molecules targeting the nucleotide sequence selected from the group consisting of SEQ ID NO:29, 30, 31 and 32. for an SUV420H1 or SUV420H2 gene. The double-stranded molecule of the present invention may be directed to a single target SUV420H1 or SUV420H2 gene sequence or may be directed to a plurality of target SUV420H1 or SUV420H2 gene sequences.

A double-stranded molecule of the present invention targeting the SUV420H1 or SUV420H2 gene includes isolated polynucleotides that contain any of the target sequences selected from the SUV420H1 or SUV420H2 gene sequence and/or complementary sequences to the target sequence. Examples of polynucleotides targeting an SUV420H1 or SUV420H2 gene include those containing the sequence corresponding to SEQ ID NO: 29, 30, 31 and 32, and/or complementary sequences to these nucleotide sequences.

In an embodiment, a double-stranded molecule is composed of two polynucleotides, one polynucleotide has a sequence corresponding to a target sequence, i.e., sense strand, and another polynucleotide has a complementary sequence to the target sequence, i.e., antisense strand. The sense strand polynucleotide and the antisense strand polynucleotide hybridize to each other to form a double-stranded molecule. Examples of such double-stranded molecules include dsRNA and dsD/R-NA . In an another embodiment, a double-stranded molecule is composed of a polynucleotide that has both a sequence corresponding to a target sequence, i.e., sense strand, and a complementary sequence to the target sequence, i.e., antisense strand. Generally, the sense strand and the antisense strand are linked by an intervening strand, and hybridize to each other to form a hairpin loop structure. Examples of such double-stranded molecule include shRNA and shD/R-NA. In some embodiments, double-stranded molecules targeting the SUV420H1 or SUV420H2 gene may have a sequence selected from among SEQ ID NOs:29, 30, 31 and 32. as a target sequence. Accordingly, examples of the double-stranded molecules of the present invention include polynucleotides that hybridize to each other at a sequence corresponding to SEQ ID NO: 29, 30, 31 or 32 and a complementary sequence thereto, and a polynucleotide that has a sequence corresponding to SEQ ID NO: 29, 30, 31 or 32 and a complementary sequence thereto.

In other words, a double-stranded molecule of the present invention comprises a sense strand polynucleotide having a nucleotide sequence of the target sequence and an anti-sense strand polynucleotide having a nucleotide sequence complementary to the target sequence, and both of polynucleotides hybridize to each other to form the double-stranded molecule. In the double-stranded molecule comprising the polynucleotides, a part of the polynucleotide of either or both of the strands may be RNA, and when the target sequence is defined with a DNA sequence, the nucleotide “t” within the target sequence and complementary sequence thereto is replaced with “u”. Alternatively, a part of the polynucleotide of either or both of the strands may be DNA, and when the target sequence is defined with an RNA sequence, the nucleotide “u” within the target sequence and complementary sequence thereto is replaced with “t”.

In one embodiment of the present invention, such a double-stranded molecule of the present invention comprises a stem-loop structure, composed of the sense and antisense strands. The sense and antisense strands may be joined by a loop. Accordingly, the present invention also provides the double-stranded molecule comprising a single polynucleotide containing both the sense strand and the antisense strand linked or flanked by an intervening single-strand.

However, the present invention is not limited to these examples, and minor modifications in the aforementioned nucleic acid sequences are acceptable so long as the modified molecule retains the ability to suppress the expression of an SUV420H1 or SUV420H2 gene. Herein, the phrase “minor modification” as used in connection with a nucleic acid sequence indicates one, two or several substitutions, deletions, additions or insertions of nucleotide(s) to the sequence. In the context of the present invention, the term “several” as applied to nucleotide substitutions, deletions, additions and/or insertions may mean 3 to 7, 3 to 5, 3 or 4, or 3 nucleic acid residues.

According to the present invention, a double-stranded molecule of the present invention can be tested for its ability to inhibit the SUV420H1 or SUV420H2 gene expression using the methods utilized in the Examples. In the Examples herein below, double-stranded molecules composed of sense strands of some portions of mRNA of the SUV420H1 or SUV420H2 gene and antisense strands complementary thereto were tested in vitro for their ability to decrease production of an SUV420H1 or SUV420H2 gene product in various cancer cell lines (e.g., using SW780, RT4, A549,LC319 and SBC-5) according to standard methods. For example, reduction in the SUV420H1 or SUV420H2 gene product in cells transfected with the candidate double-stranded molecule as compared to that in cells transfected with no oligonucleotide or control siRNA (e.g., siRNA against EGFP) can be detected by, e.g., RT-PCR using primers for an SUV420H1 or SUV420H2 mRNA such as the primers provided under Example: “Quantitative Real-time PCR”. Candidate target sequences which decrease the production of the SUV420H1 or SUV420H2 gene product in vitro cell-based assays can then be tested for their inhibitory effects on cell growth. Target sequences which inhibit cell growth in an in vitro cell-based assay may then be tested for their in vivo ability using animals with cancer, e.g., nude mouse xenograft models, to confirm decreased production of the SUV420H1 or SUV420H2 product and decreased cancer cell growth.

When the polynucleotide contained in double-stranded molecule is RNA or derivatives thereof, base “t” should be replaced with “u” in the nucleotide sequences. Thus, as used herein, the phrase “a sequence corresponding to a target sequence” refers to a nucleotide sequence in which base “t”s of the target sequence are replaced with “u”s in RNA or derivatives thereof, or a nucleotide sequence in which base “u”s of the target sequence are replaced with “t”s in DNA or derivatives thereof. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other in the same manner. Generally, complementary polynucleotide sequences hybridize under appropriate conditions to form stable duplexes containing few or no mismatches. Furthermore, the isolated double-stranded molecule of the present invention can form a double-stranded molecule or hairpin loop structure by the hybridization of the sense strand and antisense strand. In one embodiment, such double-stranded molecules contain no more than 1 mismatch for every 10 matches. In another embodiment, where the strands of the duplex are fully complementary, such double-stranded molecules contain no mismatches.

The polynucleotide may be less than 4562 or 2711 nucleotides in length for SUV420H1, or less than 2318 nucleotides in length for SUV420H2. For example, the polynucleotide may be less than 500, 200, 100, 75, 50, or 25 nucleotides in length. The isolated polynucleotides of the present invention are useful for forming double-stranded molecules against the SUV420H1 or SUV420H2 gene or preparing template DNAs encoding the double-stranded molecules. When the polynucleotides are used for forming double-stranded molecules, the polynucleotide may be longer than 19 nucleotides, longer than 21 nucleotides, or have a length of between about 19 and 25 nucleotides. Accordingly, the present invention provides the double-stranded molecules comprising a sense strand and an antisense strand, wherein the sense strand comprises a nucleotide sequence corresponding to a target sequence. In some embodiments, the sense strand hybridizes with antisense strand at the target sequence to form the double-stranded molecule having between 19 and 25 nucleotide pairs in length.

The double-stranded molecule serves as a guide for identifying homologous sequences in mRNA for the RISC complex, when the double-stranded molecule is introduced into cells. The identified target RNA is cleaved and degraded by the nuclease activity of Dicer, through which the double-stranded molecule eventually decreases or inhibits production (expression) of the polypeptide encoded by the RNA. Thus, a double-stranded molecule of the present invention can be defined by its ability to generate a single-strand that specifically hybridizes to the mRNA of the SUV420H1 or SUV420H2 gene under stringent conditions. Herein, the portion of the mRNA that hybridizes with the single-strand generated from the double-stranded molecule is referred to as “target sequence” or “target nucleic acid” or “target nucleotide”. In the present invention, the nucleotide sequence of the “target sequence” can be shown using not only the RNA sequence of the mRNA, but also the DNA sequence of cDNA synthesized from the mRNA, or the genomic sequence of one or more exons.

The double-stranded molecules of the present invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increase or decrease affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced with deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the present invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on either or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. In some embodiments, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule may contain as much DNA as possible, whereas to induce inhibition of the target gene expression, the molecule may be required to contain RNA within a range to induce sufficient inhibition of the expression.

As an example of a chimera type double-stranded molecule, an upstream partial region (i.e., a region flanking to the target sequence or complementary sequence thereof within the sense or antisense strands) of the double-stranded molecule is RNA. In some embodiments, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in some embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention may include following combinations. sense strand:

5′-[- - - DNA - - - ]-3′

3′-(RNA)-[DNA]-5′

:antisense strand,

sense strand:

5′-(RNA)-[DNA]-3′

3′-(RNA)-[DNA]-5′

:antisense strand, and

sense strand:

5′-(RNA)-[DNA]-3′

3′-( - - - RNA - - - )-5′

:antisense strand.

The upstream partial region may be a domain composed of 9 to 13 nucleotides counted from the terminus of the target sequence or complementary sequence thereto within the sense or antisense strands of the double-stranded molecules. Moreover, examples of such chimera type double-stranded molecules include those having a strand length of 19 to 21 nucleotides in which at least the upstream half region (5′ side region for the sense strand and 3′ side region for the antisense strand) of the double-stranded molecule is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes a sense strand containing a sequence corresponding to the target sequence and an antisense containing a complementary sequence corresponding to the target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′ or 5′-[A′]-[B]-[A]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotide sequences of SEQ ID NOs: 29 and 30 for SUV420H1, and SEQ ID NOs: 31 and 32 for SUV420H2.

The present invention is not limited to these examples, and the target sequence in [A] may be modified sequences from these examples so long as the double-stranded molecule retains the ability to suppress the expression of the targeted SUV420H1 or SUV420H2 gene. The region [A] hybridizes to [A′] to form a loop composed of the region [B]. The intervening single-stranded portion [B], i.e., loop sequence may be 3 to 23 nucleotides in length. The loop sequence, for example, can be selected from among the following sequences (www.ambion.com/techlib/tb/tb_{—}506.html). Furthermore, a loop sequence consisting of 23 nucleotides also provides active siRNA (Jacque J M et al., Nature 2002 Jul. 25, 418(6896): 435-8, Epub 2002 Jun. 26):

### CCC, CCACC, or CCACACC: Jacque J M et al., Nature 2002 Jul. 25, 418(6896): 435-8, Epub 2002 Jun. 26;

### UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; or

### UUCAAGAGA: Dykxhoorn D M et al., Nat Rev Mol Cell Biol 2003 Jun., 4(6): 457-67.

Exemplary double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence of SEQ ID NO: 29)
GAGUUCUGCGAGUGUUACA-[B]-UGUAACACUCGCAGAACUC;
(for target sequence of SEQ ID NO: 30)
GAAAUUAUUCAAAGAACAU-[B]-AUGUUCUUUGAAUAAUUUC.
(for target sequence of SEQ ID NO: 31)
GGAUCUGAGCCCUGACCCU-[B]-AGGGUCAGGGCUCAGAUCC.
(for target sequence of SEQ ID NO: 32)
GCAUAGCUCUGACCCUGGA-[B]-UCCAGGGUCAGAGCUAUGC.

Furthermore, in order to enhance the inhibition activity of the double-stranded molecules, several nucleotides can be added to 3′end of the sense strand and/or antisense strand of the target sequence, as 3′ overhangs. Examples of nucleotides consisting a 3′ overhang include “t” and “u”, but are not limited thereto. The number of nucleotides to be added may be at least 1, or 2, and generally 2 to 10, or 2 to 5. The added nucleotides form (a) single strand(s) at the 3′ end of the sense strand and/or antisense strand of the double-stranded molecule. In cases where double-stranded molecules consists of a single polynucleotide to form a hairpin loop structure, a 3′ overhang sequence may be added to the 3′ end of the single polynucleotide.

The method for preparing the double-stranded molecule is not particularly limited and includes chemical synthetic methods known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of at least about 3:7, about 4:6, or as a substantially equimolar amount (i.e., a molar ratio of about 5:5). Next, the mixture is heated to a temperature at which double-stranded molecules dissociate and then is gradually cooled down. The annealed double-stranded polynucleotide can be purified by generally employed methods known in the art. Examples of purification methods include methods utilizing agarose gel electrophoresis. Remaining single-stranded polynucleotides may be optionally removed by, e.g., degradation with appropriate enzyme.

Alternatively, the double-stranded molecules may be transcribed intracellularly by cloning the coding sequence into a vector containing a regulatory sequence that directs the expression of the double-stranded molecule in an adequate cell (e.g., an RNA pol III transcription unit from the small nuclear RNA (snRNA) U6 or the human H1 RNA promoter) adjacent to the coding sequence. The regulatory sequences flanking the coding sequences of double-stranded molecule may be identical or different, such that their expression can be modulated independently, or in a temporal or spatial manner. Details of vectors which are capable of producing the double-stranded molecules are described bellow.

Vectors Containing a Double-Stranded Molecule of the Present Invention:

Also included in the present invention are vectors containing one or more of the double-stranded molecules described herein, and a cell containing such a vector.

Specifically, the present invention provides the following vector of [1] to [11].

[1] A vector, encoding a double-stranded molecule that, when introduced into a cell expressing either or both of an SUV420H1 and SUV420H2 gene, inhibits the expression of the SUV420H1 or SUV420H2 gene and cell proliferation, such molecules composed of a sense strand and an antisense strand complementary thereto, hybridized to each other to form the double-stranded molecule.

[2] The vector of [1], wherein the double-stranded molecule acts on mRNA of SUV420H1 or SUV420H2, matching a target sequence selected from among SEQ ID NOs: 29, 30, 31 and 32;

[3] The vector of [1] or [2], wherein the sense strand contains a nucleotide sequence corresponding to a target sequence selected from among SEQ ID NOs: 29, 30, 31 and 32;

[4] The vector of any one of [1] to [3], encoding the double-stranded molecule, wherein the sense strand of the double-stranded molecule hybridizes with the antisense strand at the target sequence to form the double-stranded molecule having a length of less than about 100 nucleotide pairs in length;

[5] The vector of [4], encoding the double-stranded molecule, wherein the sense strand of the double-stranded molecule hybridizes with the antisense strand at the target sequence to form the double-stranded molecule having a length of less than about 75 nucleotide pairs in length;

[6] The vector of [5], encoding the double-stranded molecule, wherein the sense strand of the double-stranded molecule hybridizes with the antisense strand at the target sequence to form the double-stranded molecule having a length of less than about 50 nucleotide pairs in length;

[7] The vector of [6] encoding the double-stranded molecule, wherein the sense strand of the double-stranded molecule hybridizes with the antisense strand at the target sequence to form the double-stranded molecule having a length of less than about 25 nucleotide pairs in length;

[8] The vector of [7], encoding the double-stranded molecule, wherein the sense strand of the double-stranded molecule hybridizes with the antisense strand at the target sequence to form the double-stranded molecule having between about 19 and about 25 nucleotide pairs in length;

[9] The vector of any one of [1] to [8], wherein the double-stranded molecule is composed of a single polynucleotide having both the sense and antisense strands linked by an intervening single-strand;

[10] The vector of [9], encoding the double-stranded molecule having the general formula 5′-[A]-[B]-[A′]-3′ or 5′-[A′]-[B]-[A]-3′, wherein [A] is the sense strand containing a sequence corresponding to a target sequence selected from among SEQ ID NOs: 29, 30, 31 and 32, [B] is the intervening single-strand composed of 3 to 23 nucleotides, and [A′] is the antisense strand containing a sequence complementary to [A]; and

[11] The vector of any one of [1] to [10], wherein the double-stranded molecule contains one or two 3′ overhang(s).

A vector of the present invention may encode a double-stranded molecule of the present invention in an expressible form. Herein, the phrase “in an expressible form” indicates that the vector, when introduced into a cell, will express the molecule. In one embodiment, the vector includes regulatory elements necessary for expression of the double-stranded molecule. Accordingly, the expression vector may encode the nucleic acid sequences of the double-stranded molecules of the present invention and be adapted for expression of said double-stranded molecules. Such vectors of the present invention may be used for producing the present double-stranded molecules, or directly as an active ingredient for treating cancer.

Vectors of the present invention can be produced, for example, by cloning the sequence encoding the double-stranded molecule into an expression vector so that regulatory sequences are operatively-linked to the coding sequences of the double-stranded molecule in a manner to allow expression (by transcription of the DNA molecule) of both strands (Lee N S et al., Nat Biotechnol 2002 May, 20(5): 500-5). For example, an RNA molecule that is the antisense strand to mRNA is transcribed by a first promoter (e.g., a promoter sequence flanking to the 3′ end of the cloned DNA) and an RNA molecule that is the sense strand to the mRNA is transcribed by a second promoter (e.g., a promoter sequence flanking to the 5′ end of the cloned DNA). After transcribed, the sense and antisense strands hybridize to each other in vivo to generate double-stranded molecule constructs for silencing of the gene. Alternatively, two vector constructs respectively encoding the sense and antisense strands of the double-stranded molecule may be utilized to respectively express the sense and antisense strands and then form a double-stranded molecule. Furthermore, the cloned sequence may encode a construct having a secondary structure (e.g., hairpin); namely, a single transcript of a vector contains both the sense and complementary antisense sequences of the target gene.

The present invention contemplates a vector that includes each or both of a combination of polynucleotides, including a sense strand nucleic acid and an antisense strand nucleic acid, wherein the antisense strand includes a nucleotide sequence which is complementary to said sense strand, wherein the transcripts of said sense strand and said antisense strand hybridize to each other to form said double-stranded molecule, and wherein said vector, when introduced into a cell expressing the SUV420H1 or SUV420H2gene, inhibits expression of said gene.

The vectors of the present invention may also be equipped so as to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the double-stranded molecule and thereby suppresses the proliferation of the cell. Another example of a useable vector includes Bacille Calmette Guerin (BCG) vectors. BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods Of Inhibiting Cancer Cell Growth and Treating Cancer Using Double-Stranded Molecules:

The present invention provides methods for inhibiting cancer cell growth, e.g., bladder cancer, cervical cancer, osteosarcoma, lung cancer, soft tissue tumor, breast cancer, chronic myelogenous leukemia (CML), esophageal cancer and gastric cancer cell growth, by inducing dysfunction of an SUV420H1 or SUV420H2 gene via inhibiting the expression of SUV420H1 or SUV420H2. The SUV420H1 or SUV420H2 gene expression can be inhibited by any of the aforementioned double-stranded molecules of the present invention which specifically target of the SUV420H1 or SUV420H2 gene or the vectors of the present invention that can express any of the double-stranded molecules.

The ability of the present double-stranded molecules and vectors to inhibit cell growth of cancerous cell indicates that they can be used in methods for treating cancer, as well as treating or preventing a post-operative, secondary, or metastatic recurrence thereof. Thus, the present invention provides methods to treat patients with cancer associated with SUV420H1 or SUV420H2 overexpression, for example, bladder cancer, cervical cancer, osteosarcoma, lung cancer, soft tissue tumor, breast cancer, chronic myelogenous leukemia (CML), esophageal cancer or gastric cancer, by administering a double-stranded molecule against the SUV420H1 or SUV420H2 gene or a vector expressing the molecule. The therapeutic method of the present invention may be carried out without adverse effect because that those genes are hardly expressed in normal organs.

Specifically, the present invention provides the following methods of [1] to [40]:

[1] A method of either or both of treating and preventing cancer, or inhibiting cancer cell growth in a subject, comprising administering to a subject a pharmaceutically effective amount of a double-stranded molecule against an SUV420H1 or SUV420H2 gene or a vector encoding the double-stranded molecule, wherein the double-stranded molecule, when introduced into a cell expressing either or both of the SUV420H1 and SUV420H2 gene, inhibits the expression of the SUV420H1 or SUV420H2 gene as well as cell proliferation, the molecule comprising a sense strand and an antisense strand complementary thereto, the strands hybridized to each other to form the double-stranded molecule;

[2] The method of [1], wherein the double-stranded molecule acts at mRNA which matches a target sequence selected from among SEQ ID NOs: 29, 30, 31 and 32;

[3] The method of [1] or [2], wherein the sense strand contains the nucleotide sequence corresponding to a target sequence selected from among SEQ ID NOs: 29, 30, 31 and 32;

[4] The method of any one of [1] to [3], wherein the cancer is selected from the group consisting of bladder cancer, cervical cancer, osteosarcoma, lung cancer, soft tissue tumor, breast cancer, chronic myelogenous leukemia (CML), esophageal cancer and gastric cancer;

[5] The method of [4], wherein the cancer is selected from the group consisting of bladder cancer, cervical cancer, osteosarcoma, lung cancer and soft tissue tumor, when the double-stranded molecule against the SUV420H1 gene is administered to the subject;

[6] The method of [4], wherein the cancer is selected from the group consisting of bladder cancer, breast cancer, chronic myelogenous leukemia (CML), esophageal cancer, gastric cancer and lung cancer, when the double-stranded molecule against the SUV420H2 gene is administered to the subject;

[7] The method of [5], wherein the lung cancer is small-cell lung cancer (SCLC) when the double-stranded molecule against the SUV420H1 gene is administered to the subject;

[8] The method of any one of [1] to [7], wherein multiple types of the double-stranded molecules are administered;

[9] The method of any one of [1] to [3], wherein the sense strand of the double-stranded molecule hybridizes with the antisense strand at the target sequence to form the double-stranded molecule having less than about 100 nucleotide pairs in lengths;

[10] The method of [9], wherein the sense strand of the double-stranded molecule hybridizes with the antisense strand at the target sequence to form the double-stranded molecule having less than about 75 nucleotide pairs in lengths;

[11] The method of [10], wherein the sense strand of the double-stranded molecule hybridizes with the antisense strand at the target sequence to form the double-stranded molecule having less than about 50 nucleotide pairs in lengths;

[12] The method of [11], wherein the sense strand of the double-stranded molecule hybridizes with the antisense strand at the target sequence to form the double-stranded molecule having less than about 25 nucleotide pairs in lengths;

[13] The method of [12], wherein the sense strand of the double-stranded molecule hybridizes with the antisense strand at the target sequence to form the double-stranded molecule having between about 19 and about 25 nucleotide pairs in length;

[14] The method of any one of [1] to [13], wherein the double-stranded molecule is composed of a single polynucleotide containing both the sense strand and the antisense strand linked by an intervening single-strand;

[15] The method of [14], wherein the double-stranded molecule has the general formula 5′-[A]-[B]-[A′]-3′ or 5′-[A′]-[B]-[A]-3, wherein [A] is the sense strand containing a sequence corresponding to a target sequence selected from among SEQ ID NOs: 29, 30, 31 and 32, [B] is the intervening single strand composed of 3 to 23 nucleotides, and [A′] is the antisense strand containing a sequence complementary to [A];

[16] The method any one of [1] to [15], wherein the double-stranded molecule is an RNA;

[17] The method any one of [1] to [15], wherein the double-stranded molecule contains both DNA and RNA;

[18] The method of [17], wherein the double-stranded molecule is a hybrid of a DNA polynucleotide and an RNA polynucleotide;

[19] The method of [18] wherein the sense and antisense strand polynucleotides are composed of DNA and RNA, respectively;

[20] The method of [17], wherein the double-stranded molecule is a chimera of DNA and RNA;

[21] The method of [20], wherein a region flanking the 3′-end of the antisense strand, or both of a region flanking the 5′-end of sense strand and a region flanking the 3′-end of antisense strand are composed of RNA;

[22] The method of [21], wherein the flanking region is composed of 9 to 13 nucleotides;

[23] The method of any one of [1] to [22], wherein the double-stranded molecule contains one or two 3′ overhang(s);

[24] The method of any one of [1] to [23], wherein the double-stranded molecule is contained in a composition which includes, in addition to the molecule, a transfection-enhancing agent and pharmaceutically acceptable carrier.

[25] The method of [1], wherein the double-stranded molecule is encoded by a vector;

[26] The method of [25], wherein the double-stranded molecule encoded by the vector acts at mRNA which matches a target sequence selected from among SEQ ID NO: 29, 30, 31 and 32.

[27] The method of [25] or [26], wherein the sense strand of the double-stranded molecule encoded by the vector contains the sequence corresponding to a target sequence selected from among SEQ ID NOs: 29, 30, 31 and 32.

[28] The method of any one of [25] to [27], wherein the cancer is selected from the group consisting of bladder cancer, cervical cancer, osteosarcoma, lung cancer, soft tissue tumor, breast cancer, chronic myelogenous leukemia (CML), esophageal cancer and gastric cancer;

[29] The method of [28], wherein the cancer is selected from the group consisting of bladder cancer, cervical cancer, osteosarcoma, lung cancer and soft tissue tumor, when a vector that encodes a double-stranded molecule against the SUV420H1 gene is administered to the subject;

[30] The method of [28], wherein the cancer is selected from the group consisting of bladder cancer, breast cancer, chronic myelogenous leukemia (CML), esophageal cancer, gastric cancer and lung cancer, when a vector that encodes a double-stranded molecule against the SUV420H2 gene is administered to the subject;

[31] The method of [29], wherein the lung cancer is SCLC when the vector that encodes the double-stranded molecule against the SUV420H1 gene is administered to the subject

[32] The method of any one of [25] to [31], wherein multiple types of the double-stranded molecules are administered;

[33] The method of any one of [25] to [32], wherein the sense strand of the double-stranded molecule encoded by the vector has a length of less than about 100 nucleotide pairs in length;

[34] The method of [33], wherein the sense strand of the double-stranded molecule hybridizes with the antisense strand at the target sequence to form the double-stranded molecule having less than about 75 nucleotide pairs in length;

[35] The method of [34], wherein the sense strand of the double-stranded molecule hybridizes with the antisense strand at the target sequence to form the double-stranded molecule having less than about 50 nucleotide pairs in length;

[36] The method of [35], wherein the sense strand of the double-stranded molecule hybridizes with the antisense strand at the target sequence to form the double-stranded molecule having has a length of less than about 25 nucleotide pairs in length;

[37] The method of [36], wherein the sense strand of the double-stranded molecule hybridizes with the antisense strand at the target sequence to form the double-stranded molecule having between about 19 and about 25 nucleotide pairs in length;

[38] The method of any one of [25] to [37], wherein the double-stranded molecule encoded by the vector is composed of a single polynucleotide containing both the sense strand and the antisense strand linked by an intervening single-strand;

[39] The method of [38], wherein the double-stranded molecule encoded by the vector has the general formula 5′-[A]-[B]-[A′]-3′ or 5′-[A′]-[B]-[A]-3′, wherein [A] is the sense strand containing a sequence corresponding to a target sequence selected from among SEQ ID NOs: 29, 30, 31 and 32, [B] is a intervening single-strand is composed of 3 to 23 nucleotides, and [A′] is the antisense strand containing a sequence complementary to [A]; and

[40] The method of any one of [25] to [39], wherein the double-stranded molecule encoded by the vector is contained in a composition which includes, in addition to the molecule, a transfection-enhancing agent and pharmaceutically acceptable carrier.

The method of the present invention will be described in more detail below.

The growth of cells expressing an SUV420H1 or SUV420H2 gene may be inhibited by contacting the cells with a double-stranded molecule against the SUV420H1 or SUV420H2 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kinds of cancer cells may be suppressed according to the present method so long as the cells express or over-express the target gene of the double-stranded molecule of the present invention. Exemplary cancer cells include bladder cancer cells, cervical cancer cells, osteosarcoma cells, lung cancer cells, soft tissue tumor cells, breast cancer cells, chronic myelogenous leukemia (CML) cells, esophageal cancer cells and gastric cancer cells. For example, the double-stranded molecules against the SUV420H1 gene or vectors encoding them may be used to inhibit the growth of bladder cancer cells, cervical cancer cells, osteosarcoma cells, lung cancer cells (in particular, SCLC cells) or soft tissue tumor cells. For example, the double-stranded molecule against the SUV420H2 gene or vectors encoding them may be used to inhibit the growth of bladder cancer cells, breast cancer cells, chronic myelogenous leukemia (CML) cells, esophageal cancer cells, gastric cancer cells or lung cancer cells. Lung cancer may be NSCLC or SCLC. Likewise, NSCLC includes adenocarcinoma, squamous cell carcinoma (SCC) and large-cell carcinoma.

Thus, patients (subjects) suffering from or at risk of developing disease related to SUV420H1 or SUV420H2 may be treated by administering at least one of the present double-stranded molecules, at least one vector expressing at least one of the molecules or at least one composition containing at least one of the molecules or vectors. For example, patients (subjects) with bladder cancer, cervical cancer, osteosarcoma, lung cancer, soft tissue tumor, breast cancer, chronic myelogenous leukemia (CML), esophageal cancer or gastric cancer may be treated by the therapeutic methods of the present invention. In typical embodiments, the double-stranded molecules against he SUV420H1 gene or the vectors encoding them may be administered to patients with bladder cancer, cervical cancer, osteosarcoma, lung cancer (in particular, SCLC) or soft tissue tumor. In other typical embodiments, the double-stranded molecule against the SUV420H2 gene or the vectors encoding them may be administered to patients with bladder cancer, breast cancer, chronic myelogenous leukemia (CML), esophageal cancer, gastric cancer s or lung cancer. The type of cancer may be identified by standard methods according to the particular type of tumor to be diagnosed. Bladder cancer, cervical cancer, osteosarcoma, lung cancer, soft tissue tumor, breast cancer, chronic myelogenous leukemia (CML), esophageal cancer or gastric cancer may be diagnosed, for example, with known tumor markers, such as Carcinoembryonic antigen (CEA), CYFRA and pro-GRP as a lung cancer marker, and TPA as a bladder cancer marker, or with Chest X-Ray and/or Sputum Cytology. In some embodiments, patients treated by the methods of the present invention are selected by detecting the expression level of SUV420H1 or SUV420H2 gene in a biological sample from the patient (subject) by conventional methods such as RT-PCR or immunoassay. In some embodiments, before the treatment of the present invention, the biopsy specimen from the patient (subject) may be confirmed for SUV420H1 or SUV420H2 gene over-expression by methods known in the art, for example, immunohistochemical analysis or RT-PCR.

According to the present method to inhibit cancer cell growth and thereby treating cancer, when administering plural kinds of the double-stranded molecules (or vectors expressing or compositions containing the same), each of the molecules may have different structures but acts at mRNA which matches the same target sequence. Alternatively plural kinds of the double-stranded molecules may acts at mRNA which matches different target sequence of same gene or acts at mRNA which matches different target sequence of different gene. For example, the method may utilize double-stranded molecules directed to one, two or more target sequence of SUV420H1 or SUV420H2 gene. Alternatively, the method may utilize the double-stranded molecules directed to target sequences of SUV420H1 or SUV420H2 gene and other genes.

For inhibiting cancer cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is deemed “efficacious” if it leads to a clinical benefit such as, reduction in expression of an SUV420H1 or SUV420H2 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness may be determined in association with any known method for diagnosing or treating the particular tumor type.

It is understood that the double-stranded molecule of the present invention may degrade the SUV420H1 or SUV420H2 mRNA in substoichiometric amounts. Without wishing to be bound by any theory, it is believed that the double-stranded molecule of the present invention may cause degradation of the target mRNA in a catalytic manner. Thus, compared to standard cancer therapies, significantly less double-stranded molecule needs to be delivered at or near the site of cancer to exert therapeutic effect.

One skilled in the art can readily determine an effective amount of the double-stranded molecule of the present invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the present invention is an intercellular concentration at or near the cancer site of from about 1 nanomolar (nM) to about 100 nM, from about 2 nM to about 50 nM, or from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer expressing either or both of SUV420H1 and SUV420H2; for example bladder cancer, cervical cancer, osteosarcoma, lung cancer, soft tissue tumor, breast cancer, chronic myelogenous leukemia (CML), esophageal cancer or gastric cancer. In particular, a double-stranded molecule containing a target sequence selected from the mRNA sequence of SUV420H1 or SUV420H2 may be used for the treatment of bladder cancer, cervical cancer, osteosarcoma, lung cancer, soft tissue tumor, breast cancer, chronic myelogenous leukemia (CML), esophageal cancer or gastric cancer. Lung cancer may be NSCLC or SCLC. Likewise, NSCLC includes adenocarcinoma, squamous cell carcinoma (SCC) and large-cell carcinoma.

For treating cancer, the double-stranded molecule of the present invention can also be administered to a subject in combination with a pharmaceutical agent different from the double-stranded molecule. Alternatively, the double-stranded molecule of the present invention can be administered to a subject in combination with another therapeutic method designed to treat cancer. For example, the double-stranded molecule of the present invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery substance, or as a recombinant plasmid or viral vector which expresses the double-stranded molecule.

Suitable delivery substances for administration in conjunction with the double-stranded molecule include the Minis Transit TKO lipophilic substance; lipofectin; lipofectamine; cellfectin; or polycations (e.g., polylysine), or liposomes. In some embodiments of the present invention, the delivery substances are liposomes.

Liposomes can aid in the delivery of the double-stranded molecule to a particular tissue, such as lung tumor tissue, and can also increase the blood half-life of the double-stranded molecule. Liposomes suitable for use in the present invention may be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

The liposomes encapsulating the double-stranded molecule of the present invention may include a ligand molecule that can deliver the liposome to the cancer site. Ligands may include those which bind to receptors prevalent in tumor or vascular endothelial cells, such as monoclonal antibodies that bind to tumor antigens or endothelial cell surface antigens.

In some embodiments, the liposomes encapsulating the double-stranded molecule of the present invention are modified so as to avoid clearance by the mononuclear macrophage and reticuloendothelial systems, for example, by having opsonization-inhibiting moieties bound to the surface of the structure. In one embodiment, a liposome may include both opsonization-inhibiting moieties and a ligand.

Opsonization-inhibiting moieties for use in preparing liposomes are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, stealth liposomes target tissue characterized by such microvasculature defects, for example, solid tumors, which will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes modified with opsonization-inhibiting moieties can deliver the double-stranded molecule of the present invention to tumor cells.

Opsonization-inhibiting moieties suitable for modifying liposomes include water-soluble polymers with a molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization-inhibiting polymer can be a block copolymer of PEG and either a polyamino acid, polysaccharide, polyamidoamine, polyethyleneamine, or polynucleotide. The opsonization-inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments, the opsonization-inhibiting moiety is a PEG, PPG, or derivatives thereof. Liposomes modified with PEG or PEG-derivatives are sometimes called “PEGylated liposomes”.

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Vectors expressing a double-stranded molecule of the present invention are discussed above. Such vectors expressing at least one double-stranded molecule of the present invention can also be administered directly or in conjunction with a suitable delivery substance, including the Mirus Transit LT1 lipophilic substance; lipofectin; lipofectamine; cellfectin; polycations (e.g., polylysine) or liposomes. Methods for delivering recombinant viral vectors, which express a double-stranded molecule of the present invention, to an area of cancer in a patient are within the skill of the art.

The double-stranded molecule of the present invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant including a porous, non-porous, or gelatinous material); and inhalation. Generally, injections or infusions of the double-stranded molecule or vector are given at or near the site of cancer.

The double-stranded molecule of the present invention can be administered in a single dose or in multiple doses. Where the administration of the double-stranded molecule of the present invention is by infusion, the infusion can be a single sustained dose or can be delivered by multiple infusions. Generally, the double-stranded molecule is injected directly into the tissue at or near the site of cancer. In some embodiments of the present invention, multiple injections of the double-stranded molecule into the tissue at or near the site of cancer are utilized.

One skilled in the art can also readily determine an appropriate dosage regimen for administering the double-stranded molecule of the present invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, or from about seven to about ten days. In a typical dosage regimen, the double-stranded molecule may be injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

In the present invention, a cancer overexpressing SUV420H1 or SUV420H2 can be treated with at least one active ingredient selected from the group consisting of:

(a) a double-stranded molecule of the present invention,

(b) DNA encoding thereof, and

(c) a vector encoding thereof.

Accordingly, in one embodiment, the present invention provides a method of (i) diagnosing whether a subject has the cancer to be treated, and/or (ii) selecting a subject for cancer treatment, which method includes the steps of:

a) determining the expression level of SUV420H1 or SUV420H2 in cancer cells or tissue(s) obtained from a subject who is suspected to have the cancer to be treated;

b) comparing the expression level of SUV420H1 or SUV420H2 with a normal control level;

c) diagnosing the subject as having the cancer to be treated, if the expression level of SUV420H1 or SUV420H2 is increased as compared to the normal control level; and

d) selecting the subject for cancer treatment, if the subject is diagnosed as having the cancer to be treated, in step c).

Alternatively, such a method includes the steps of:

a) determining the expression level of SUV420H1 or SUV420H2 in cancer cells or tissue(s) obtained from a subject who is suspected to have the cancer to be treated;

b) comparing the expression level of SUV420H1 or SUV420H2 with a cancerous control level;

c) diagnosing the subject as having the cancer to be treated, if the expression level of SUV420H1 or SUV420H2 is similar or equivalent to the cancerous control level; and

d) selecting the subject for cancer treatment, if the subject is diagnosed as having the cancer to be treated, in step c).

Cancer includes, but is not limited to, bladder cancer, cervical cancer, osteosarcoma, lung cancer, soft tissue tumor, breast cancer, chronic myelogenous leukemia (CML), esophageal cancer or gastric cancer. Accordingly, prior to the administration of the double-stranded molecule of the present invention as active ingredient, the methods may include a step to confirm whether the expression level of SUV420H1 or SUV420H2 in the cancer cells or tissues to be treated is elevated as compared with normal cells of the same organ. Thus, in one embodiment, the present invention provides a method for treating a cancer (over)expressing SUV420H1 or SUV420H2, which method may include the steps of:

i) determining the expression level of SUV420H1 or SUV420H2 in cancer cells or tissue(s) obtained from a subject with the cancer to be treated;

ii) comparing the expression level of SUV420H1 or SUV420H2 with normal control; and

iii) administrating at least one component selected from the group consisting of

(a) a double-stranded molecule of the present invention,

(b) DNA encoding thereof, and

(c) a vector encoding thereof,

to a subject with a cancer overexpressing SUV420H1 or SUV420H2 compared with normal control. Alternatively, the present invention also provides a pharmaceutical composition comprising at least one component selected from the group consisting of:

(a) a double-stranded molecule of the present invention,
(b) DNA encoding thereof, and
(c) a vector encoding thereof,
for use in administrating to a subject having a cancer overexpressing SUV420H1 or SUV420H2. In other words, the present invention further provides a method for identifying a subject to be treated with:
(a) a double-stranded molecule of the present invention,
(b) DNA encoding thereof, or
(c) a vector encoding thereof,
which method may include the step of determining an expression level of SUV420H1 or SUV420H2 in subject-derived cancer cells or tissue(s), wherein an increase of the level compared to a normal control level of the gene indicates that the subject has cancer which may be treated with a double-stranded molecule of the present invention.

The method of treating a cancer of the present invention will be described in more detail below.

A subject to be treated by the present method is typically a mammal. Exemplary mammals include, but are not limited to, e.g., human, non-human primate, mouse, rat, dog, cat, horse, and cow.

According to the present invention, the expression level of SUV420H1 or SUV420H2 in cancer cells or tissues obtained from a subject is determined. The expression level can be determined at the transcription (nucleic acid) product level, using methods known in the art. For example, hybridization methods (e.g., Northern hybridization), a chip or an array, probes, RT-PCR can be used to determine the transcription product level of SUV420H1 or SUV420H2.

Alternatively, the translation product may be detected for the treatment of the present invention. For example, the quantity of observed protein (SEQ ID NO: 24 and 26, for SUV420H1, SEQ ID No:28 for SUV420H2) may be determined.

As another method to detect the expression level of SUV420H1 or SUV420H2 gene based on its translation product, the intensity of staining may be measured via immunohistochemical analysis using an antibody against the SUV420H1 or SUV420H2 protein. Namely, in this measurement, strong staining indicates increased presence/level of the protein and, at the same time, high expression level of SUV420H1 or SUV420H2 gene.

Methods for detecting or measuring the SUV420H1 or SUV420H2 polypeptide and/or polynucleotide encoding thereof can be exemplified as described above (Method of detecting or diagnosing cancer).

Compositions Containing Double-Stranded Molecules:

In addition to the above, the present invention also provides pharmaceutical compositions that include at least one of the double-stranded molecules of the present invention or the vectors coding for the molecules.

In the context of the present invention, the term “composition” is used to refer to a product that include the specified ingredients in the specified amounts, as well as any product that results, directly or indirectly, from combination of the specified ingredients in the specified amounts. Such terms, when used in relation to the modifier “pharmaceutical” (as in “pharmaceutical composition”), are intended to encompass products including a product that includes the active ingredient(s), and any inert ingredient(s) that make up the carrier, as well as any product that results, directly or indirectly, from combination, complexation or aggregation of any two or more of the ingredients, or from dissociation of ones or more of the ingredients, or from other types of reactions or interactions of one or more of the ingredients. Accordingly, in the context of the present invention, the term “pharmaceutical composition” refers to any product made by admixing a molecule or compound of the present invention and a pharmaceutically or physiologically acceptable carrier.

The phrase “pharmaceutically acceptable carrier” or “physiologically acceptable carrier”, as used herein, means a pharmaceutically or physiologically acceptable material, composition, substance or vehicle, including but not limited to, a liquid or solid filler, diluent, excipient, solvent or encapsulating material.

The term “active ingredient” herein refers to a substance in composition that is biologically or physiologically active. Particularly, in the context of pharmaceutical composition, the term “active ingredient” refers to a substance that shows an objective pharmacological effect. For example, in case of pharmaceutical compositions for use in the treatment or prevention of cancer, active ingredients in the agents or compositions may lead to at least one biological or physiologically action on cancer cells and/or tissues directly or indirectly. Preferably, such action may include reducing or inhibiting cancer cell growth, damaging or killing cancer cells and/or tissues, and so on. Before being formulated, the “active ingredient” may also be referred to as “bulk”, “drug substance” or “technical product”.

Specifically, the present invention provides the following compositions [1] to [40]:

[1] A composition for either or both of treating and preventing cancer, and inhibiting cancer cell growth, wherein the cancer cell and the cancer expresses an SUV420H1 or SUV420H2 gene, including a pharmaceutically effective amount of an isolated double-stranded molecule against the SUV420H1 or SUV420H2 gene or pharmaceutically acceptable salt thereof, or a vector encoding the double-stranded molecule, which molecule is composed of a sense strand and an antisense strand complementary thereto, hybridized to each other to form the double-stranded molecule, wherein the double-stranded molecule, when introduced into a cell expressing either or both of the SUV420H1 and SUV420H2 gene, inhibits the expression of the SUV420H1 or SUV420H2 gene as well as cell proliferation, and pharmaceutically acceptable carrier;

[2] The composition of [1], wherein the double-stranded molecule acts at mRNA which matches a target sequence selected from among SEQ ID NOs: 29, 30, 31 and 32;

[3] The composition of [1] or [2], wherein the double-stranded molecule, wherein the sense strand contains a nucleotide sequence corresponding to a target sequence selected from among SEQ ID NOs: 29, 30, 31 and 32;

[4] The composition of any one of [1] to [3], wherein the cancer is selected from the group consisting of bladder cancer, cervical cancer, osteosarcoma, lung cancer, soft tissue tumor, breast cancer, chronic myelogenous leukemia (CML), esophageal cancer and gastric cancer;

[5] The composition of [4], wherein the cancer is selected from the group consisting of bladder cancer, cervical cancer, osteosarcoma, lung cancer and soft tissue tumor, when the double-stranded molecule against the SUV420H1 gene is included in the composition;

[6] The composition of [4], wherein the cancer is selected from the group consisting of bladder cancer, breast cancer, chronic myelogenous leukemia (CML), esophageal cancer, gastric cancer and lung cancer, when the double-stranded molecule against the SUV420H2 gene is included in the composition;

[7] The composition of [5], wherein the lung cancer is SCLC;

[8] The composition of any one of [1] to [7], wherein the composition contains multiple types of the double-stranded molecules;

[9] The composition of any one of [1] to [8], wherein the sense strand of the double-stranded molecule hybridizes with the antisense strand at the target sequence to form the double-stranded molecule having less than about 100 nucleotide pairs in length;

[10] The composition of [9], wherein the sense strand of the double-stranded molecule hybridizes with the antisense strand at the target sequence to form the double-stranded molecule having less than about 75 nucleotide pairs in length;

[11] The composition of [10], wherein the sense strand of the double-stranded molecule hybridizes with the antisense strand at the target sequence to form the double-stranded molecule having less than about 50 nucleotide pairs in length;

[12] The composition of [11], wherein the sense strand of the double-stranded molecule hybridizes with the antisense strand at the target sequence to form the double-stranded molecule having less than about 25 nucleotide pairs in length;

[13] The composition of [12], wherein the sense strand of the double-stranded molecule hybridizes with the antisense strand at the target sequence to form the double-stranded molecule having between about 19 and about 25 nucleotide pairs in length;

[14] The composition of any one of [1] to [13], wherein the double-stranded molecule is composed of a single polynucleotide containing the sense strand and the antisense strand linked by an intervening single-strand;

[15] The composition of [14], wherein the double-stranded molecule has the general formula 5′-[A]-[B]-[A′]-3′ or 5′-[A′]-[B]-[A]-3′, wherein [A] is the sense strand sequence containing a nucleotide sequence corresponding to a target sequence selected from among SEQ ID NOs: 29, 30, 31 and 32, [B] is the intervening single-strand consisting of 3 to 23 nucleotides, and [A′] is the antisense strand containing a nucleotide sequence complementary to [A];

[16] The composition of any one of [1] to [15], wherein the double-stranded molecule is an RNA;

[17] The composition of any one of [1] to [15], wherein the double-stranded molecule is DNA and/or RNA;

[18] The composition of [17], wherein the double-stranded molecule is a hybrid of a DNA polynucleotide and an RNA polynucleotide;

[19] The composition of [18], wherein the sense and antisense strand polynucleotides are composed of DNA and RNA, respectively;

[20] The composition of [17], wherein the double-stranded molecule is a chimera of DNA and RNA;

[21] The composition of [20], wherein a region flanking the 3′-end of the antisense strand, or both of a region flanking the 5′-end of sense strand and a region flanking the 3′-end of antisense strand are composed of RNA;

[22] The composition of [21], wherein the flanking region is composed of 9 to 13 nucleotides;

[23] The composition of any one of [1] to [22], wherein the double-stranded molecule contains one or two 3′ overhang(s);

[24] The composition of any one of [1] to [23], wherein the composition includes a transfection-enhancing agent;

[25] The composition of [1], wherein the double-stranded molecule is encoded by a vector;

[26] The composition of [25], wherein the double-stranded molecule encoded by the vector acts at mRNA which matches a target sequence selected from among SEQ ID NOs: 29, 30, 31 and 32;

[27] The composition of [25] or [26], wherein the sense strand of the double-stranded molecule encoded by the vector contains the nucleotide sequence corresponding to a target sequence selected from among SEQ ID NOs: 29, 30, 31 and 32;

[28] The composition of any one of [25] to [27], wherein the cancer is selected from the group consisting of bladder cancer, cervical cancer, osteosarcoma, lung cancer, soft tissue tumor, breast cancer, chronic myelogenous leukemia (CML), esophageal cancer and gastric cancer;

[29] The composition of [28], wherein the cancer is selected from the group consisting of bladder cancer, cervical cancer, osteosarcoma, lung cancer and soft tissue tumor, when the double-stranded molecule against the SUV420H1 gene encoded by the vector is included in the composition;

[30] The composition of [28], wherein the cancer is selected from the group consisting of bladder cancer, breast cancer, chronic myelogenous leukemia (CML), esophageal cancer, gastric cancer and lung cancer, when the double-stranded molecule against the SUV420H2 gene encoded by the vector is included in the composition;

[31] The composition of [29], wherein the lung cancer is SCLC;

[32] The composition of any one of [25] to [28], wherein the composition contains the vector encodes multiple types of double-stranded molecules or multiple types of vectors, each of the vectors encoding a different double-stranded molecule;

[33] The composition of any one of [25] to [32], wherein the sense strand of the double-stranded molecule hybridizes with the antisense strand at the target sequence to form the double-stranded molecule having less than about 100 nucleotide pairs in length;

[34] The composition of [33], wherein the sense strand of the double-stranded molecule hybridizes with the antisense strand at the target sequence to form the double-stranded molecule having less than about 75 nucleotide pairs in length;

[35] The composition of [34], wherein the sense strand of the double-stranded molecule hybridizes with the antisense strand at the target sequence to form the double-stranded molecule having less than about 50 nucleotide pairs in length;

[36] The composition of [35], wherein the sense strand of the double-stranded molecule hybridizes with the antisense strand at the target sequence to form the double-stranded molecule having less than about 25 nucleotide pairs in length;

[37] The composition of [36], wherein the sense strand of the double-stranded molecule hybridizes with the antisense strand at the target sequence to form the double-stranded molecule having between about 19 and about 25 nucleotide pairs in length;

[38] The composition of any one of [25] to [37], wherein the double-stranded molecule encoded by the vector is composed of a single polynucleotide containing both the sense strand and the antisense strand linked by an intervening single-strand;

[39] The composition of [38], wherein the double-stranded molecule has the general formula 5′-[A]-[B]-[A′]-3′ or 5′-[A′]-[B]-[A]-3′, wherein [A] is the sense strand containing a nucleotide sequence corresponding to a target sequence selected from among SEQ ID NOs: 29, 30, 31 and 32, [B] is a intervening single-strand composed of 3 to 23 nucleotides, and [A′] is the antisense strand containing a nucleotide sequence complementary to [A];

[40] The composition of any one of [25] to [39], wherein the composition includes a transfection-enhancing agent; and

Additional details of the compositions of the present invention are described below.

Compositions of the present invention may be formulated as pharmaceutical compositions, according to techniques known in the art. Compositions of the present invention may be characterized as being at least sterile and pyrogen-free. As used herein, “pharmaceutical compositions” include formulations for human and veterinary use. Thus, the compositions may be used as pharmaceuticals for humans and other mammals, such as mice, rats, guinea-pigs, rabbits, cats, dogs, sheep, pigs, cattle, monkeys, baboons, and chimpanzees.

In the context of the present invention, suitable pharmaceutical formulations of the present invention include those suitable for oral, rectal, nasal, topical (including buccal, sub-lingual, and transdermal), vaginal or parenteral (including intramuscular, subcutaneous and intravenous) administration, or for administration by inhalation or insufflation. Other formulations include implantable devices and adhesive patches that release a therapeutic agent. When desired, the above-described formulations may be adapted to give sustained release of the active ingredient.

Methods for preparing the compositions of the present invention are within the skill in the art, for example as described in Remington's Pharmaceutical Science, 17th ed., Mack Publishing Company, Easton, Pa. (1985), the entire disclosure of which is herein incorporated by reference.

The compositions of the present invention contain at least one of the double-stranded molecules of the present invention or vectors encoding them (e.g., 0.1 to 90% by weight), or pharmaceutically acceptable salts of the molecules, mixed with a pharmaceutically acceptable carrier. Typical pharmaceutically acceptable carrier are water, buffered water, normal saline, 0.4% saline, 0.3% glycine, hyaluronic acid and the like.

According to the present invention, the composition may contain multiple types of the double-stranded molecules, each of the molecules may be directed to different target sequences of SUV420H1 or SUV420H2, or target sequences of SUV420H1 or SUV420H2 and other genes. For example, the composition may contain double-stranded molecules directed to one, two or more target sequences of SUV420H1 or SUV420H2. Alternatively, for example, the composition may contain double-stranded molecules directed to a target sequence of SUV420H1 or SUV420H2 and double-stranded molecules directed to target sequences of other genes.

Furthermore, the present composition may contain a vector coding for one or more double-stranded molecules. For example, the vector may encode one, two or several kinds of the double-stranded molecules of the present invention. Alternatively, the present composition may contain multiple vectors, each of the vectors coding for a different double-stranded molecule.

Moreover, the double-stranded molecules of the present invention may be contained as liposomes encapsulating the molecules in the present composition. See under the item of “Methods of inhibiting cancer cell growth and treating cancer using double-stranded molecules” for details of liposomes.

Compositions of the present invention may also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Compositions of the present invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, solid compositions for oral administration can include any of carriers and excipients listed above and 10-95%, or 25-75%, of one or more double-stranded molecule of the present invention. Compositions for aerosol (inhalational) administration can include 0.01-20% by weight, or 1-10% by weight, of one or more double-stranded molecule of the present invention encapsulated in a liposome as described above, and propellant. For intranasal delivery, compositions may include carriers such as lecithin.

In addition to the above, the present composition may contain other pharmaceutical active ingredients so long as they do not inhibit the in vivo function of the double-stranded molecules of the present invention. For example, the composition may contain chemotherapeutic agents conventionally used for treating cancers.

The pharmaceutical compositions may also contain other active ingredients such as antimicrobial agents, immunosuppressants or preservatives. Furthermore, it should be understood that, in addition to the ingredients particularly mentioned above, the formulations of this invention may include other agents conventional in the art having regard to the type of formulation in question; for example, those suitable for oral administration may include flavoring agents.

Alternatively, the present invention further provides the double-stranded nucleic acid molecules of the present invention for use in treating a cancer expressing either or both of the SUV420H1 and SUV420H2 gene.

In another embodiment, the present invention also provides the use of the double-stranded molecules of the present invention in manufacturing a pharmaceutical composition or medicament for treating a cancer characterized by the expression of SUV420H1 or SUV420H2. For example, the present invention relates to the use of double-stranded molecule inhibiting the expression of an SUV420H1 or SUV420H2 gene in a cell, which molecule includes a sense strand and an antisense strand complementary thereto, hybridized to each other to form the double-stranded molecule, and targets a nucleotide sequence selected from among SEQ ID NOs: 29, 30, 31 and 32, for manufacturing a pharmaceutical composition for treating cancer expressing either or both of SUV420H1 and SUV420H2, such as bladder cancer, cervical cancer, osteosarcoma, lung cancer, soft tissue tumor, breast cancer, chronic myelogenous leukemia (CML), esophageal cancer and gastric cancer.

Alternatively, the present invention further provides a method or process for manufacturing a pharmaceutical composition for treating cancer characterized by the expression of either or both of SUV420H1 and SUV420H2, wherein the method or process includes a step for formulating a pharmaceutically or physiologically acceptable carrier with a double-stranded nucleic acid molecule inhibiting the expression of SUV420H1 or SUV420H2 in a cell, which over-expresses the gene, which molecule includes a sense strand and an antisense strand complementary thereto, hybridized to each other to form the double-stranded molecule and targets a nucleotide sequence selected from among SEQ ID NOs: 29, 30, 31 and 32 as active ingredients.

In another embodiment, the present invention also provides a method or process for manufacturing a pharmaceutical composition for treating cancer characterized by the expression of either or both of SUV420H1 and SUV420H2, wherein the method or process includes a step for admixing an active ingredient with a pharmaceutically or physiologically acceptable carrier, wherein the active ingredient is a double-stranded nucleic acid molecule inhibiting the expression of SUV420H1 or SUV420H2 in a cell, which over-expresses the gene, which molecule includes a sense strand and an antisense strand complementary thereto, hybridized to each other to form the double-stranded molecule and targets a nucleotide sequence selected from among SEQ ID NOs: 15, 17, 19 and 21.

### Method of Detecting or Diagnosing Cancer:

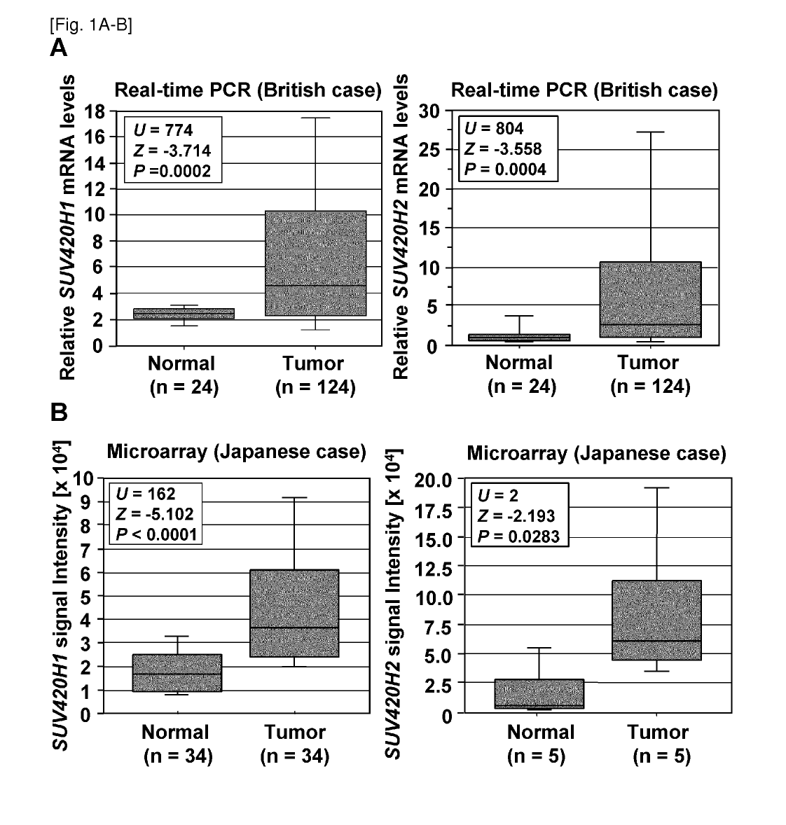

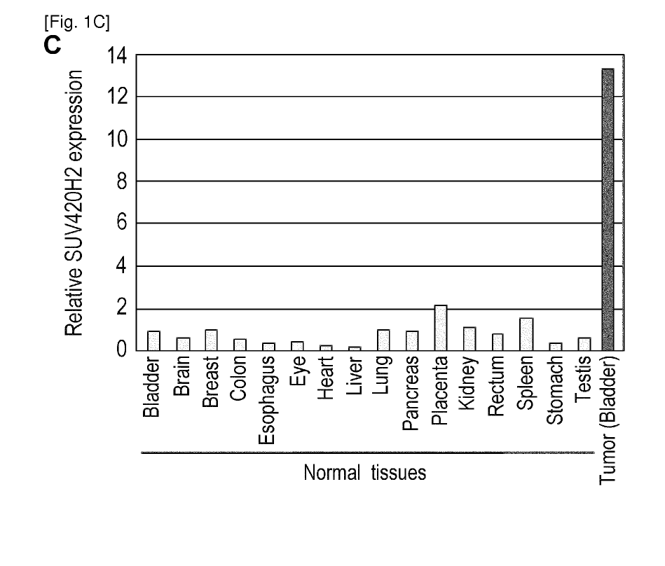

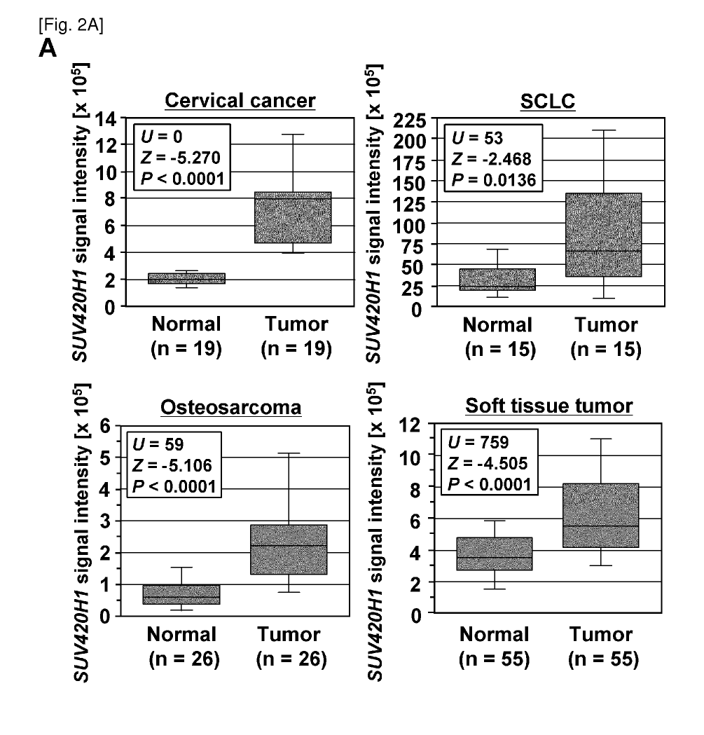

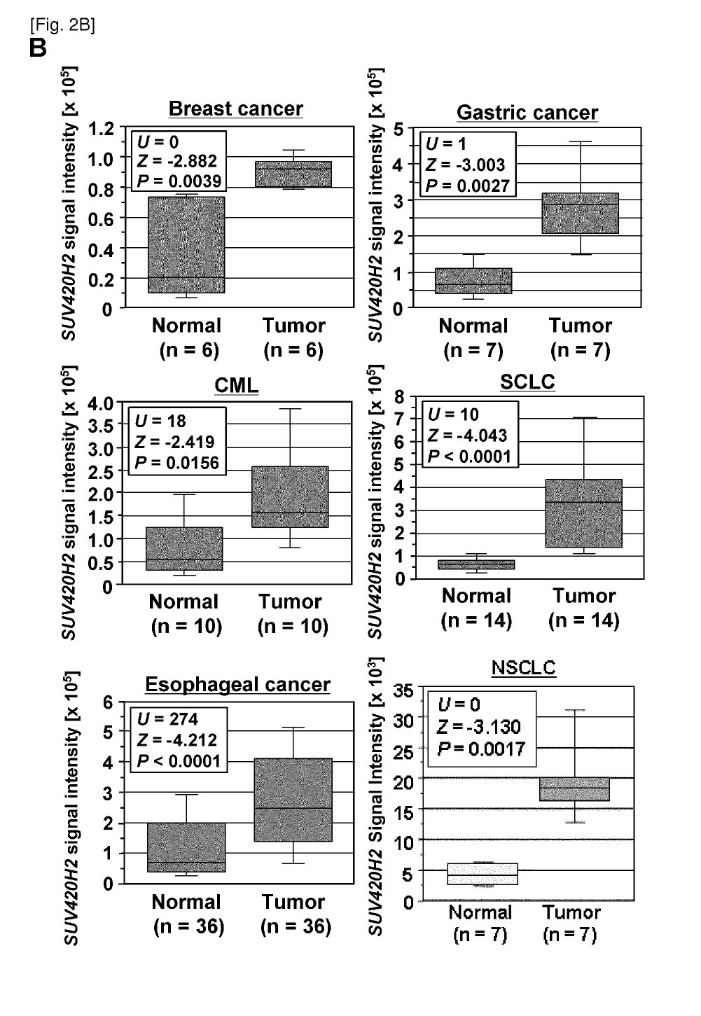

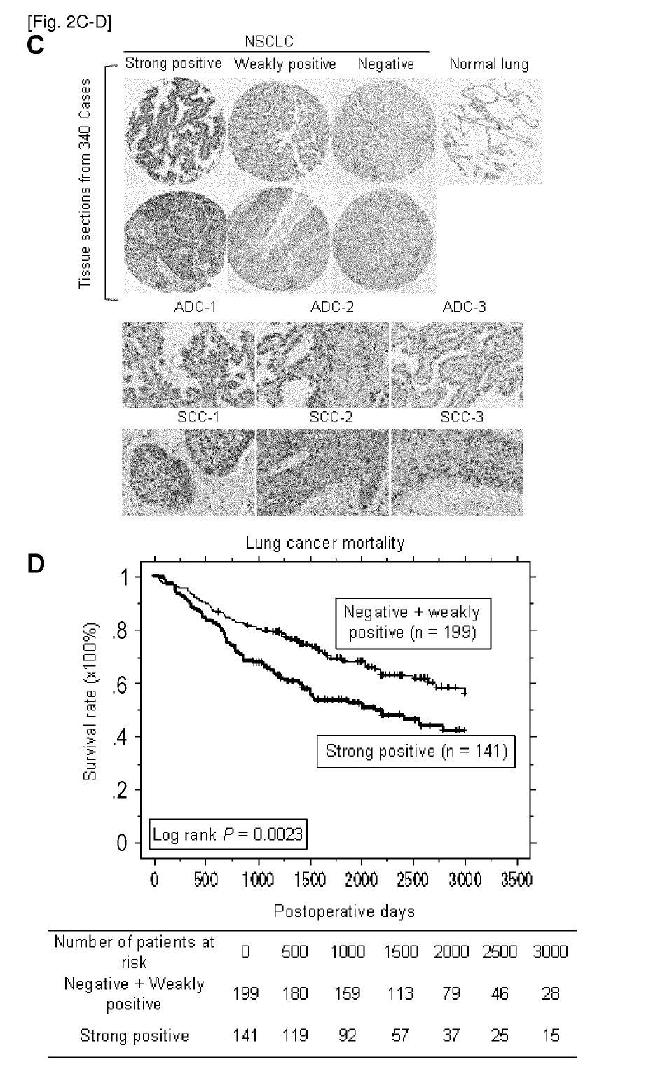

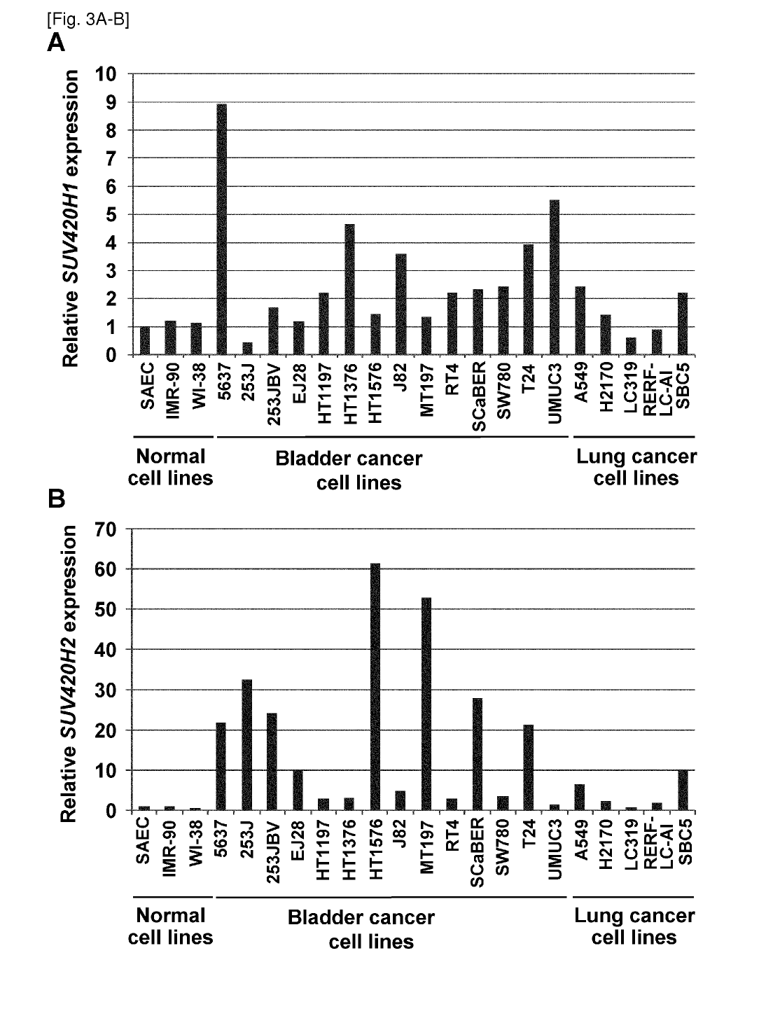

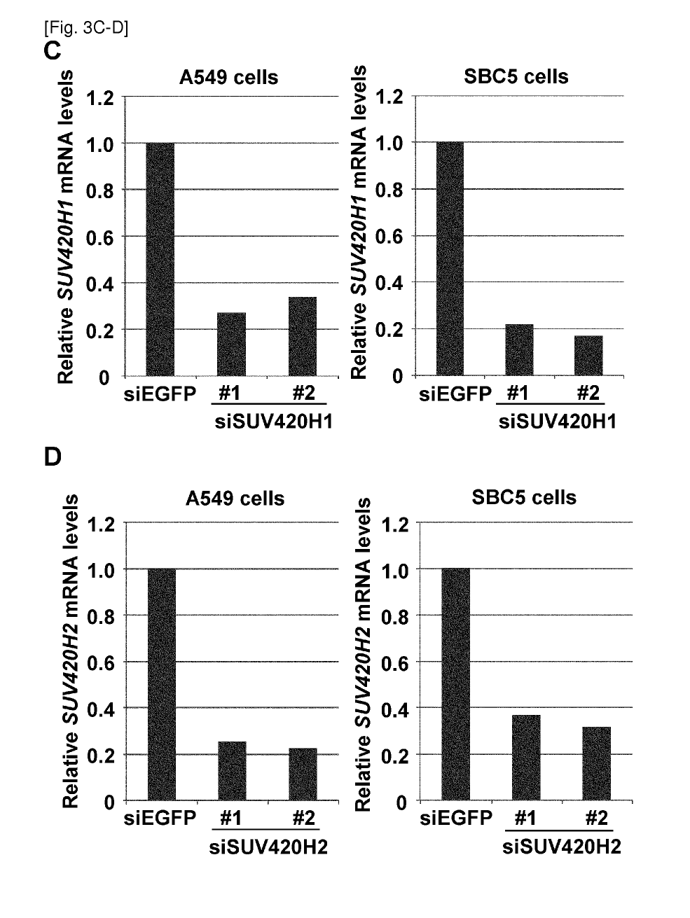

The expressions of SUV420H1 was found to be specifically elevated in bladder cancer (FIGS. 1A and 1B), cervical cancer, osteosarcoma, lung cancer (FIG. 2A), bladder cancer cell lines and lung cancer cell lines (FIG. 3A). The expression of SUV420H2 was found to be specifically elevated in bladder cancer (FIGS. 1A and 1B), breast cancer, chronic myelogenous leukemia (CML), esophageal cancer, gastric cancer, lung cancer (FIG. 2B), bladder cancer cell lines and lung cancer cell lines (FIG. 3B). Therefore, the genes identified herein as well as their transcription and translation products find diagnostic utility as markers for cancer, e.g. bladder cancer, cervical cancer, osteosarcoma, lung cancer, soft tissue tumor, breast cancer, chronic myelogenous leukemia (CML), esophageal cancer and gastric cancer. Also, by measuring the expression level of SUV420H1 or SUV420H2 in a subject-derived biological sample, cancer, e.g. bladder cancer, cervical cancer, osteosarcoma, lung cancer, soft tissue tumor, breast cancer, chronic myelogenous leukemia (CML), esophageal cancer and gastric cancer, can be diagnosed. Specifically, the present invention provides a method for detecting or diagnosing cancer, e.g. bladder cancer, cervical cancer, osteosarcoma, lung cancer, soft tissue tumor, breast cancer, chronic myelogenous leukemia (CML), esophageal cancer and gastric cancer by determining the expression level of SUV420H1 or SUV420H2 in a subject-derived biological sample. Lung cancers that can be diagnosed by the present method include SCLC and NSCLC. Likewise, NSCLC includes adenocarcinoma, squamous cell carcinoma (SCC) and large-cell carcinoma.

According to the present invention, an intermediate result for examining the condition of a subject may be provided. Such intermediate result may be combined with additional information to assist a doctor, nurse, or other practitioner to diagnose that a subject suffers from the disease. That is, the present invention provides a diagnostic marker SUV420H1 or SUV420H2 for examining cancer. Alternatively, the present invention provides a method for detecting or identifying cancer cells in a subject-derived tissue, for example bladder tissue sample, uterine tissue sample, bone tissue sample, lung tissue sample, soft tissue sample, breast tissue sample, myeloid tissue (bone-marrow tissue) sample, esophageal tissue sample and gastric tissue sample, said method including the step of determining the expression level of the SUV420H1 or SUV420H2 gene in a subject-derived tissue, wherein an increase in said expression level as compared to a normal control level of said gene indicates the presence or suspicion of cancer cells in the tissue. Such result may be combined with additional information to assist a doctor, nurse, or other healthcare practitioner in diagnosing a subject as afflicted with the disease. In other words, the present invention may provide a doctor with useful information to diagnose disease a subject as afflicted with the disease. For example, according to the present invention, when there is doubt regarding the presence of cancer cells in the tissue obtained from a subject, clinical decisions can be reached by considering the expression level of the SUV420H1 or SUV420H2 gene, plus a different aspect of the disease including tissue pathology, levels of known tumor marker(s) in blood, and clinical course of the subject, etc. For example, some well-known diagnostic lung tumor markers in blood are TAP, ACT, BFP, CA19-9, CA50, CA72-4, CA130, CEA, KMO-1, NSE, SCC, SP1, Span-1, TPA, CSLEX, SLX, STN and CYFRA, bladder tumor markers in blood are BTA, TAP and so on. Namely, in this particular embodiment of the present invention, the outcome of the gene expression analysis serves as an intermediate result for further diagnosis of a subject's disease state.

Specifically, the present invention provides the following methods [1] to [13]:

[1] A method of detecting or diagnosing cancer in a subject, comprising determining an expression level of SUV420H1 or SUV420H2 gene in a subject-derived biological sample, wherein an increase of said level compared to a normal control level of said gene indicates that said subject suffers from or is at risk of developing cancer, wherein the expression level is determined by any one of method selected from the group consisting of:

(a) detecting the mRNA of SUV420H1 or SUV420H2;

(b) detecting the protein encoded by the SUV420H1 or SUV420H2 gene; and

(c) detecting the biological activity of the protein encoded by the SUV420H1 or SUV420H2 gene.

[2] The method of [1], wherein the expression level is at least 10% greater than the normal control level;

[3] The method of [1] or [2], wherein the cancer is selected from the group consisting of bladder cancer, cervical cancer, osteosarcoma, lung cancer, soft tissue tumor, breast cancer, chronic myelogenous leukemia (CML), esophageal cancer and gastric cancer;

[4] The method of [3], wherein the cancer is selected from the group consisting of bladder cancer, cervical cancer, osteosarcoma, lung cancer and soft tissue tumor when the expression level of the SUV420H1 gene is determined;

[5] The method of [3], wherein the cancer is selected from the group consisting of bladder cancer, breast cancer, chronic myelogenous leukemia (CML), esophageal cancer, gastric cancer and lung cancer when the expression level of the SUV420H2 gene is determined;

[6] The method of [4], wherein the lung cancer is SCLC;

[7] The method of any one of [1] to [6], wherein the expression level is determined by detecting hybridization of a probe to the mRNA of the gene;

[8] The method of any one of [1] to [6], wherein the expression level is determined by detecting the binding of an antibody against the protein encoded by the gene;

[9] The method of any one of [1] to [8], wherein the subject-derived biological sample includes a biopsy specimen, sputum, blood, pleural effusion or urine;

[10] The method of any one of [1] to [9], wherein the subject-derived biological sample includes an epithelial cell;

[11] The method of [10], wherein the subject-derived biological sample includes a cancer cell; and

[12] The method of Mt wherein the subject-derived biological sample includes a cancerous epithelial cell.

[13] The method of [3], wherein when the cancer is bladder cancer, the subject-derived biological sample is a bladder tissue sample derived from the subject, when the cancer is cervical cancer, the subject-derived biological sample is an uterine tissue sample derived from the subject, when the cancer is osteosarcoma, the subject-derived biological sample is bone tissue derived from the subject, when the cancer is lung cancer, the subject-derived biological sample is a lung tissue sample derived from the subject, when the cancer is a soft tissue tumor, the subject-derived biological sample is a soft tissue sample derived from the subject, when the cancer is breast cancer, the subject-derived biological sample is a breast tissue sample derived from the subject, when the cancer is chronic myelogenous leukemia (CML), the subject-derived biological sample is a myeloid tissue (bone-marrow tissue) sample derived from the subject, when the cancer is esophageal cancer, the subject-biological sample is esophageal tissue derived from the subject, and when the cancer is gastric cancer, the subject-derived biological sample is a gastric tissue sample derived from the subject.

The method of diagnosing cancer (e.g., bladder cancer, cervical cancer, osteosarcoma, lung cancer, soft tissue tumor, breast cancer, chronic myelogenous leukemia (CML), esophageal cancer and gastric cancer) will be described in more detail below.

A subject to be diagnosed by the present method is typically a mammal. Exemplary mammals include, but are not limited to, e.g., human, non-human primate, mouse, rat, dog, cat, horse, and cow.

In some embodiments of the present invention, a biological sample is collected from a subject to be diagnosed to perform the cancer diagnosis. Any biological material can be used as a biological sample for the determination so long as it includes or is suspected of including the transcription or translation products of the SUV420H1 or SUV420H2 gene. The biological samples may include, but are not limited to, bodily tissues desired for diagnosis or suspected to be cancerous, and fluids, such as a biopsy specimen, blood, sputum, pleural effusion and urine. For example, the biological sample may contain a cell population including an epithelial cell, or a cancerous epithelial cell or an epithelial cell derived from tissue suspected to be cancerous. Further, if necessary, the cell may be purified from the obtained bodily tissues and fluids, and then used as the biological sample.

For example, according to the present invention, suitable cancers for diagnosis or detection include bladder cancer, cervical cancer, osteosarcoma, lung cancer, soft tissue tumor, breast cancer, chronic myelogenous leukemia (CML), esophageal cancer and gastric cancer. In order to diagnose or detect theses cancers, a subject-derived biological sample may be collected from following organs:

bladder: for bladder cancer,

uterine: for cervical cancer

bone: for osteosarcoma

lung: for lung cancer

soft tissue: for soft tissue tumor

breast: for breast cancer

myeloid (bone-marrow): for chronic myelogenous leukemia (CML)

esophagus: for esophageal cancer

stomach: for gastric cancer

According to methods of the present invention, the expression level of SUV420H1 or SUV420H2 in a subject-derived biological sample is determined and then correlated to a particular healthy or disease state by comparison to that in a control sample. The expression level can be determined at the transcription product (nucleic acid) level, using methods known in the art. For example, the mRNA of SUV420H1 or SUV420H2 may be quantified using probes by hybridization methods (e.g., Northern hybridization). The detection may be carried out on a chip or an array. An array may be used for detecting the expression level of a plurality of genes (e.g., various cancer specific genes) including SUV420H1 or SUV420H2. Those skilled in the art can prepare such probes utilizing the sequence information of the SUV420H1 gene (e.g., SEQ ID NO: 23 and 25; GenBank accession number: NM_{—}017635.3 and NM_{—}016028.4) and the SUV420H2 gene (e.g., SEQ ID NO: 27; GenBank accession number: NM_{—}032701.3). For example, the cDNA of SUV420H1 or SUV420H2 may be used as the probes. If necessary, the probe may be labeled with a suitable label, such as dyes, fluorescent labels and isotopes, and the expression level of the gene may be detected as the intensity of the hybridized labels.

Furthermore, the transcription product of SUV420H1 or SUV420H2 may be quantified using primers by amplification-based detection methods (e.g., RT-PCR). Such primers can also be prepared based on the available sequence information of the genes. For example, the primers or probes used in the Example (SEQ ID NOs: 5, 6, 7, 8, 9, 10, 11 and 12) may be employed for the detection by RT-PCR, but the present invention is not restricted thereto.

Specifically, a probe or primer used for the present method hybridizes under stringent, moderately stringent, or low stringent conditions to the mRNA of SUV420H1 or SUV420H2. As also defined above, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of SUV420H1 or SUV420H2 protein may be determined. A method for determining the quantity of the protein as the translation product includes immunoassay methods that use an antibody specifically recognizing the protein or a fragment thereof. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to an SUV420H1 or SUV420H2 protein. Methods to prepare these kinds of antibodies for the detection of proteins are well known in the art, and any method may be employed in the present invention to prepare such antibodies and equivalents thereof.

As another method to detect the expression level of an SUV420H1 or SUV420H2 gene based on its translation product, the intensity of staining may be observed via immunohistochemical analysis using an antibody against an SUV420H1 or SUV420H2 protein, or a fragment thereof. Namely, the observation of strong staining indicates increased presence of the protein and at the same time high expression level of an SUV420H1 or SUV420H2 gene.

Furthermore, the quantity of SUV420H1 or SUV420H2 protein can be determined by measuring the biological activity of the SUV420H1 or SUV420H2 protein, such as histone methylation. As described above, SUV420H1 and SUV420H2 are histone methyltransferases that catalyze di- and trimethylation of histone H4K20 which is characteristic of pericentric heterochromatin. Therefore, histone methyltransferase activity is useful for quantification of SUV420H1 or SUV420H2 protein based on its biological activity. The methylation level of histone (especially, histone H4K20) can be determined by methods well known in the art.

Alternatively, cell proliferation enhancing activity may be used as a biological activity of SUV420H1 or SUV420H2 protein. According to the present invention, inhibiting the expression of SUV420H1 or SUV420H2 gene leads to suppression cell growth in bladder cancer and lung cancer cells, therefore, SUV420H1 or SUV420H2 protein promotes cell proliferation. For determining the cell proliferation enhancing activity of SUV420H1 or SUV420H2 protein, the cell is cultured in the presence of a biological sample, and then by detecting the speed of proliferation, or by measuring the cell cycle or the colony forming ability, the cell proliferation enhancing activity of the biological sample can be determined.

In the context of the present invention, methods for detecting or identifying cancer in a subject or cancer cells in a subject-derived biological sample begin with a determination of SUV420H1 or SUV420H2 gene expression level. Once determined, using any of the aforementioned techniques, this value is as compared to a control level.

In the context of the present invention, the phrase “control level” refers to the expression level of a test gene detected in a control sample and encompasses both a normal control level and a cancer control level. The phrase “normal control level” refers to a level of gene expression detected in a normal healthy individual or in a population of individuals known not to be suffering from cancer. A normal individual is one with no clinical symptom of bladder cancer, cervical cancer, osteosarcoma, lung cancer, soft tissue tumor, breast cancer, chronic myelogenous leukemia (CML), esophageal cancer and/or gastric cancer. A normal control level can be determined using a normal cell obtained from a non-cancerous tissue. A “normal control level” may also be the expression level of a test gene detected in a normal healthy tissue or cell of an individual or population known not to be suffering from bladder cancer, cervical cancer, osteosarcoma, lung cancer, soft tissue tumor, breast cancer, chronic myelogenous leukemia (CML), esophageal cancer and/or gastric cancer. On the other hand, the phrase “cancer control level” refers to an expression level of a test gene detected in the cancerous tissue or cell of an individual or population suffering from bladder cancer, cervical cancer, osteosarcoma, lung cancer, soft tissue tumor, breast cancer, chronic myelogenous leukemia (CML), esophageal cancer and/or gastric cancer. An increase in the expression level of SUV420H1 or SUV420H2 detected in a subject-derived sample as compared to a normal control level indicates that the subject (from which the sample has been obtained) suffers from or is at risk of developing bladder cancer, cervical cancer, osteosarcoma, lung cancer, soft tissue tumor, breast cancer, chronic myelogenous leukemia (CML), esophageal cancer and/or gastric cancer. In the context of the present invention, the subject-derived biological sample may be any tissues obtained from test subjects, e.g., patients suspected of having cancer. For example, tissues may include epithelial cells. More particularly, tissues may be epithelial cells collected from a suspected cancerous area. Alternatively, the expression level of SUV420H1 or SUV420H2 in a sample can be compared to a cancer control level of SUV420H1 or SUV420H2 gene. A similarity between the expression level of a sample and the cancer control level indicates that the subject (from which the sample has been obtained) suffers from or is at risk of developing cancer. When the expression levels of other cancer-related genes are also measured and compared, a similarity in the gene expression pattern between the sample and the reference that is cancerous indicates that the subject is suffering from or at a risk of developing cancer.

The control level may be determined at the same time as the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of an SUV420H1 or SUV420H2 gene in samples from subjects whose disease state are known. Furthermore, the control level can be from a database of expression patterns from previously tested cells. Moreover, according to an aspect of the present invention, the expression level of the SUV420H1 or SUV420H2 gene in a biological sample may be compared to multiple control levels, which control levels are determined from multiple reference samples. In one embodiment, the methods of the present invention use a control level determined from a reference sample derived from a tissue type similar to that of the patient-derived biological sample. Moreover, a standard value of the expression levels of the SUV420H1 or SUV420H2 gene in a population with a known disease state may be used. The standard value may be obtained by any method known in the art. For example, a range of mean+/−2 S.D. or mean+/−3 S.D. may be used as standard value.

To improve the accuracy of the diagnosis, the expression level of other cancer-associated genes, for example, genes known to be differentially expressed in bladder cancer, cervical cancer, osteosarcoma, lung cancer, soft tissue tumor, breast cancer, chronic myelogenous leukemia (CML), esophageal cancer and/or gastric cancer may also be determined, in addition to the expression level of the SUV420H1 or SUV420H2 gene. Furthermore, in the case where the expression levels of multiple cancer-related genes are compared, a similarity in the gene expression pattern between the sample and the reference that is cancerous indicates that the subject is suffering from or at a risk of developing bladder cancer, cervical cancer, osteosarcoma, lung cancer, soft tissue tumor, breast cancer, chronic myelogenous leukemia (CML), esophageal cancer and/or gastric cancer.

In the context of the present invention, gene expression levels are deemed to be “altered” or “increased” when the gene expression changes or increases by, for example, 10%, 25%, or 50% from, or at least 0.1 fold, at least 0.2 fold, at least 0.5 fold, at least 2 fold, at least 5 fold, or at least 10 fold or more compared to a control level. Accordingly, the expression level of bladder cancer, cervical cancer, osteosarcoma, lung cancer, soft tissue tumor, breast cancer, chronic myelogenous leukemia (CML), esophageal cancer and/or gastric cancer marker genes, including the SUV420H1 and SUV420H2 gene, in a biological sample can be considered to be increased if it increases from a control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

Method for Assessing the Prognosis of Cancer:

The present invention further relates to the discovery that SUV420H2 expression is significantly associated with poorer prognosis of patients. Thus, the present invention also provides a method for determining or assessing the prognosis of a subject with cancer by determining the expression level of the SUV420H2 gene in a subject-derived biological sample; comparing the determined expression level to a control level; and assessing or determining the prognosis of the subject based on the comparison. In typical embodiments, the prognosis of the subject with lung cancer (e.g., NSCLC) is assessed or determined by the method of the present invention.

In addition, the expression level of the SUV420H2 gene before and after a treatment can be compared according to the present method to assess the efficacy of the treatment and/or monitor disease status (e.g., progression, regression, or remission). Specifically, the expression level detected in a subject-derived biological sample after a treatment (i.e., post-treatment level) may be compared to the expression level detected in a biological sample obtained prior to treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the formation of tumor or retards, prevents, or alleviates at least one clinical symptom of the disease. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice.

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The phrase “assessing (or determining) the prognosis” refer to the ability of predicting, forecasting or correlating a given detection or measurement with a future outcome of cancer of the subject (e.g., malignancy, likelihood of curing cancer, survival, and the like). For example, a determination of the expression level of the SUV420H2 gene over time enables a predicting of an outcome for the subject (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer progression, particularly cancer recurrence, metastatic spread and disease relapse. The present method for assessing or determining prognosis is intended to be used clinically in making decisions concerning treatment modalities, including therapeutic intervention, diagnostic criteria such as disease staging, and disease monitoring and surveillance for metastasis or recurrence of neoplastic disease.

In the context of the present invention, subject-derived biological samples may be any samples derived from the subject to be assessed so long as they include or are suspected of including the transcription or translation product of the SUV420H2 gene. In typical embodiments, subject-derived biological samples include lung cells (cells obtained from lung). For example, a lung tissue sample collected from a cancerous area can be used as a subject-derived biological sample. Biological samples may be cells purified from tissue collected from test subjects. Alternatively, subject-derived biological samples may include bodily fluids such as sputum, blood, serum, or plasma. The biological samples may be obtained from a subject at various time points, including before, during, and/or after a treatment.

According to the present invention, the higher expression level of the SUV420H2 gene determined in a subject-derived biological sample, the poorer prognosis for post-treatment remission, recovery, and/or survival and the higher likelihood of poor clinical outcome. Thus, according to the present method, the “control level” used for comparison may be, for example, the expression level of the SUV420H2 gene detected before any kind of treatment in an individual or a population of individuals who showed good or positive prognosis of cancer, after the treatment, which herein is referred to as “good prognosis control level”. Alternatively, the “control level” may be the expression level of the SUV420H2 gene detected before any kind of treatment in an individual or a population of individuals who showed poor or negative prognosis of cancer, after the treatment, which herein will be referred to as “poor prognosis control level”. The “control level” may be a single expression pattern derived from a single reference population or from a plurality of expression patterns. Thus, the control level may be determined based on the expression level of the SUV420H2 gene detected before any kind of treatment in a patient of cancer, or a population of the subjects whose disease state (good or poor prognosis) is known. In some embodiment, the standard value of the expression levels of the SUV420H2 gene in a subject group with a known disease state or prognosis may be used. The standard value may be obtained by any method known in the art. For example, a range of mean+/−2 S.D. or mean+/−3S.D. may be used as standard value.

The control level may be determined at the same time with the test biological sample by using a sample(s) previously collected and stored before any kind of treatment from cancer subject(s) (control or control group) whose disease state (good prognosis or poor prognosis) are known.

Alternatively, the control level may be determined by a statistical method based on the results obtained by analyzing the expression level of the SUV420H2 gene in samples previously collected and stored from a control group. Furthermore, the control level can be from a database of expression patterns from previously tested cells. Moreover, in an aspect of the present invention, the expression level of the

SUV420H2 gene in a subject-derived biological sample may be compared to multiple control levels, such as control levels determined in multiple reference samples. Generally, a control level determined in a reference sample derived from a tissue type similar to that of the subject-derived biological sample.

According to the present invention, a similarity between the expression level of the SUV420H2 gene determined in a subject-derived biological sample and a good prognosis control level indicates a favorable prognosis or good prognosis of the subject. Likewise, an increase in the expression level of the SUV420H2 gene determined in a subject-derived biological sample as compared to the good prognosis control level indicates less favorable, poor prognosis for post-treatment remission, recovery, survival, and/or clinical outcome. On the other hand, a decrease in the expression level of the SUV420H2 gene determined in a subject-derived biological sample as compared to a poor prognosis control level indicates a favorable prognosis or good prognosis of the subject. Likewise, a similarity between the two levels indicates less favorable, poor prognosis for post-treatment remission, recovery, survival, and/or clinical outcome. In the context of the present invention, a lung cancer cell(s) obtained from a subject who showed good, or poor prognosis of cancer after treatment is a preferable biological sample for good, or poor prognosis control level, respectively.

The expression level of the SUV420H2 gene in a subject-derived biological sample can be considered to be altered when the expression level differs from the control level by more than 1.0, 1.5, 2.0, 5.0, 10.0, or more fold.

The difference between the expression level determined in a test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression level of the SUV420H2 gene.

The expression level may be determined by detecting the gene products in a subject-derived biological sample using techniques well known in the art. The gene products detected by the present method include both the transcription and translation products, such as mRNA and protein.

For instance, the transcription product of the SUV420H2 gene can be detected by hybridization, e.g., Northern blot hybridization analyses, that use a SUV420H2 gene probe to the gene transcript. The detection may be carried out on a chip or an array. An array may be used for detecting the expression level of a plurality of genes including the SUV420H2 gene. As another example, amplification-based detection methods, such as reverse-transcription based polymerase chain reaction (RT-PCR) which use primers specific to the SUV420H2 gene may be employed for the detection (see Example). The SUV420H2 gene-specific probe or primers may be designed and prepared using conventional techniques by referring to the whole sequence of the SUV420H2 gene (SEQ ID NO: 27). For example, the primers (SEQ ID NOs: 9, 10, 11 and 12) used in the Example may be employed for the detection by RT-PCR, but the present invention is not restricted thereto.

Specifically, a probe or primer used for the present method hybridizes under stringent, moderately stringent, or low stringent conditions to the mRNA of the SUV420H2 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the assessment or determination of the prognosis. For example, the quantity of the SUV420H2 protein may be determined. A method for determining the quantity of the protein as the translation product includes immunoassay methods that use an antibody specifically recognizing the SUV420H2 protein, or a fragment thereof. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the SUV420H2 protein. Methods to prepare these kinds of antibodies for the detection of proteins are well known in the art, and any method may be employed in the present invention to prepare such antibodies and equivalents thereof.

As another method to detect the expression level of the SUV420H2 gene based on its translation product, the intensity of staining may be observed via immunohistochemical analysis using an antibody against SUV420H2 protein, or a fragment thereof. Namely, the observation of strong staining indicates increased presence of the SUV420H2 protein and at the same time high expression level of the SUV420H2 gene.

Furthermore, the quantity of SUV420H2 protein can be determined by measuring the biological activity of the SUV420H2 protein, such as histone methylation. As described above, SUV420H2 is a histone methyltransferase that catalyzes di- and trimethylation of histone H4K20. Therefore, histone methyltransferase activity is useful for quantification of SUV420H2 protein based on its biological activity. The methylation level of histone can be determined by methods well known in the art.

Furthermore, the SUV420H2 protein is known to have a cell proliferation enhancing activity. Therefore, the expression level of the SUV420H2 gene can be determined using such cell proliferation enhancing activity as an index. For example, cells that express SUV420H2 are prepared and cultured in the presence of a subject-derived biological sample, and then by detecting the speed of proliferation, or by measuring the cell cycle or the colony forming ability the cell proliferating activity of the subject-derived biological sample can be determined.

Moreover, in addition to the expression level of the SUV420H2 gene, the expression level of other prognostic gene markers, for example, genes which expression are known to be associated with cancer prognosis may also be determined to improve the accuracy of the assessment. Examples of such other lung cancer prognostic gene markers include those described in WO2009/028580 and WO 2005/090603, the contents of which are incorporated by reference herein.

Alternatively, according to the present invention, an intermediate result may also be provided in addition to other test results for assessing the prognosis of a subject. Such intermediate result may assist a doctor, nurse, or other practitioner to assess, determine, monitor or estimate the progress and/or prognosis of a subject. Additional information that may be considered, in combination with the intermediate result obtained by the present invention, to assess prognosis includes clinical symptoms and physical conditions of a subject.

In other words, the expression level of the SUV420H2 gene is useful prognostic marker for assessing, predicting or determining the prognosis of a subject suffering from cancer. Therefore, the present invention also provides a method for detecting prognostic marker for assessing, predicting or determining the prognosis of a subject suffering from cancer, which includes steps of:

a) detecting or determining an expression level of an SUV420H2 gene in a subject-derived biological sample, and

b) correlating the expression level detected or determined in step a) with the prognosis of the subject.

In particular, according to the present invention, an increased expression level as compared to the control level is indicative of potential or suspicion of poor prognosis (poor survival).

In the context of the present invention, the subject to be assessed for the prognosis of cancer may be mammals, including human, non-human primate, mouse, rat, dog, cat, horse, and cow.

A Kit for Diagnosing Cancer, Assessing or Determining the Prognosis of Cancer, or Monitoring the Efficacy of Cancer Therapy:

The present invention provides a kit for diagnosing or detecting cancer or predisposition for developing cancer. The present invention also provides a kit for assessing or determining the prognosis of cancer, or monitoring the efficacy of a cancer therapy. In the context of the present invention, examples of cancers include bladder cancer, cervical cancer, osteosarcoma, lung cancer, soft tissue tumor, breast cancer, chronic myelogenous leukemia (CML), esophageal cancer and gastric cancer. In typical embodiments, cancer to be diagnosed or detected is selected from the group consisting of bladder cancer, cervical cancer, osteosarcoma, lung cancer and soft tissue tumor, when the expression level of the SUV420H1 gene is determined. In typical embodiments, cancer to be diagnosed or detected is selected from the group consisting of bladder cancer, breast cancer, chronic myelogenous leukemia (CML), esophageal cancer, gastric cancer and lung cancer, when the SUV420H2 gene is determined. The lung cancer may be SCLC or NSCLC. In typical embodiments, cancer to be assessed or determined the prognosis is lung cancer.

Specifically, the kit includes at least one reagent for detecting the expression of the SUV420H1 or SUV420H2 gene in a patient-derived biological sample, which reagent may be selected from the group of:

(a) a reagent for detecting an mRNA of an SUV420H1 or SUV420H2gene;

(b) a reagent for detecting an SUV420H1 or SUV420H2 protein; and

(c) a reagent for detecting a biological activity of an SUV420H1 or SUV420H2 protein.

Suitable reagents for detecting mRNA of the SUV420H1 or SUV420H2 gene include nucleic acids that specifically bind to or identify the SUV420H1 or SUV420H2 mRNA, such as oligonucleotides which have a complementary sequence to a part of the SUV420H1 or SUV420H2 mRNA. These kinds of oligonucleotides are exemplified by primers and probes that are specific to the SUV420H1 or SUV420H2 mRNA. These kinds of oligonucleotides may be prepared based on methods well known in the art. If needed, the reagent for detecting the SUV420H1 or SUV420H2 mRNA may be immobilized on a solid matrix. Moreover, more than one reagent for detecting the SUV420H1 or SUV420H2 mRNA may be included in the kit.

A probe or primer of the present invention is typically a substantially purified oligonucleotide. The oligonucleotide typically includes a region of nucleotide sequence that hybridizes under stringent conditions to at least about 2000, 1000, 500, 400, 350, 300, 250, 200, 150, 100, 50, or 25 bases of consecutive sense strand nucleotide sequence of a nucleic acid including an SUV420H1 or SUV420H2 sequence, or an anti sense strand nucleotide sequence of a nucleic acid including an SUV420H1 or SUV420H2 sequence, or of a naturally occurring mutant of these sequences. In particular, for example, in one embodiment, an oligonucleotide having 5-50 nucleotides in length can be used as a primer for amplifying the genes, to be detected. In another embodiment, mRNA or cDNA of an SUV420H1 or SUV420H2 gene can be detected with an oligonucleotide probe or primer of a specific size, generally 15-30 bases in length. In some embodiments, the length of the oligonucleotide probe or primer can be selected from 15-25 bases. Assay procedures, devices, or reagents for the detection of gene by using such oligonucleotide probe or primer are well known (e.g. oligonucleotide microarray or PCR). In these assays, probes or primers can also include tag or linker sequences. Further, probes or primers can be modified with a detectable label or affinity ligand to be captured. Alternatively, in hybridization based detection procedures, a polynucleotide having a few hundred (e.g., about 100-200) bases to a few kilo (e.g., about 1000-2000) bases in length can also be used for a probe (e.g., northern blotting assay or cDNA microarray analysis).

On the other hand, suitable reagents for detecting the SUV420H1 or SUV420H2 protein include antibodies to the SUV420H1 or SUV420H2 protein, or fragments thereof. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the SUV420H1 or SUV420H2 protein. Methods to prepare these kinds of antibodies for the detection of proteins are well known in the art, and any method may be employed in the present invention to prepare such antibodies and equivalents thereof. Furthermore, the antibody or fragment thereof may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the SUV420H1 or SUV420H2 protein may be included in the kit.

Furthermore, the biological activity can be determined by, for example, measuring methyltransferase activity, or the cell proliferation enhancing activity due to the expressed SUV420H1 or SUV420H2 protein in a subject-derived biological sample.

For example, the methyltransferase activity in a subject-derived biological sample can be determined by incubating the biological sample with a substrate capable of being methylated such as histone, and then, detecting residual methylated histone using antibody against methylated histone. Thus, the present kit may include histone (especially histone H4) and anti-methylated histone antibody. Examples of such antibodies include antibodies that bind to the methylated lysine 20 of histone H4.

On the other hand, cell proliferation enhancing activity can be determined by cultivating cells in the presence of the biological sample and then detecting the speed of proliferation, or measuring the cell cycle or the colony forming ability. Thus, the present kit can include medium and one or more containers for cultivation of cells. The kit may contain more than one of the aforementioned reagents. Furthermore, the kit may include a solid matrix and reagent for binding a probe against the SUV420H1 or SUV420H2 gene, or antibody against the SUV420H1 or SUV420H2 protein, or fragment thereof, a medium and container for culturing cells, positive and negative control samples, and a secondary antibody for detecting an antibody against the SUV420H1 or SUV420H2 protein. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be included in a container with a label. Suitable containers include bottles, vials, and test tubes. The containers may be formed from a variety of materials, such as glass or plastic.

According to an aspect of the present invention, the kit of the present invention for diagnosing cancer may further include either of positive or negative controls sample, or both. The positive control sample of the present invention may be established bladder cancer cell lines, cervical cancer cell lines, osteosarcoma cell lines, lung cancer cell lines, soft tissue tumor cell lines, breast cancer cell lines, chronic myelogenous leukemia (CML) cell lines, esophageal cancer cell lines, and/or gastric cancer cell lines. In a preferred embodiment, such cell lines are selected from the group consisting of:

bladder cancer cell lines such as 5637 253J, 253J-BV, EJ28, HT1197, HT1376, HT1576, J82, MT197, RT4, SCaBER, SW780, T24, UMUC3, and the like;

lung cancer cell lines such as A549, H2170, LC319, RERF-LC-AI, SBC5, and the like;

Alternatively, the SUV420H1 or SUV420H2 positive control samples may also be a clinical bladder cancer tissue(s), cervical cancer tissue(s), osteosarcoma tissue(s), lung cancer tissue(s), soft tissue tumor tissue(s), breast cancer tissue(s), chronic myelogenous leukemia (CML) tissue(s), esophageal cancer tissue(s), and/or gastric cancer tissue(s) obtained from a bladder cancer patient(s), cervical cancer patient(s), osteosarcoma patient(s), lung cancer patient(s), soft tissue tumor patient(s), breast cancer patient(s), chronic myelogenous leukemia (CML) patient(s), esophageal cancer patient(s), and/or gastric cancer cell lines cancer patient(s). Alternatively, positive control samples may be prepared by determined a cut-off value and preparing a sample containing an amount of an SUV420H1 or SUV420H2 mRNA or protein more than the cut-off value. Herein, the phrase “cut-off value” refers to the value dividing between a normal range and a cancerous range. For example, one skilled in the art may be determine a cut-off value using a receiver operating characteristic (ROC) curve. The present kit may include an SUV420H1 or SUV420H2 standard sample providing a cut-off value amount of an SUV420H1 or SUV420H2 mRNA or polypeptide. On the contrary, negative control samples may be prepared from non-cancerous cell lines or non-cancerous tissues such as a normal bladder tissue(s), cervical tissue(s), bone tissues, lung tissue(s), soft tissue(s), breast tissue(s), bone marrow, esophageal tissue(s), and/or gastric tissue(s), or may be prepared by preparing a sample containing an SUV420H1 or SUV420H2 mRNA or protein less than cut-off value.

As an embodiment of the present invention, when the reagent is a probe against the SUV420H1 or SUV420H2 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of SUV420H1 or SUV420H2 mRNA present in the sample. The detection sites may be configured in any suitably detectable shape and are typically in the shape of a bar or dot spanning the width of a test strip.

The kit of the present invention may further include a positive control sample, negative control sample and/or SUV420H1 or SUV420H2 standard sample. The positive control sample of the present invention may be prepared by collecting SUV420H1 or SUV420H2 positive tissue samples and then those SUV420H1 or SUV420H2 level are assayed. Alternatively, a purified SUV420H1 or SUV420H2 protein or polynucleotide may be added to SUV420H1 or SUV420H2 free sample to form the positive sample or the SUV420H1 or SUV420H2 standard.

Screening for an Anti-Cancer Substance:

In the context of the present invention, substances to be identified through the present screening methods may be any substance or composition including several substances. Furthermore, the test substance exposed to a cell or protein according to the screening methods of the present invention may be a single substance or a combination of substances. When a combination of substances is used in the methods, the substances may be contacted sequentially or simultaneously.

Alternatively, the present invention provides a method of evaluating the therapeutic effect of a test substance on treating or preventing cancer or inhibiting cancer cell growth.

Any test substances, for example, cell extracts, cell culture supernatant, products of fermenting microorganisms, extracts from marine organisms, plant extracts, purified or crude proteins, peptides, non-peptide substances, synthetic micromolecular substances (including nucleic acid constructs, such as antisense RNA, siRNA, Ribozymes, and aptamers etc.) and natural substances can be used in the screening methods of the present invention. The test substance of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-substance” library method and (5) synthetic library methods using affinity chromatography selection. The biological library methods using affinity chromatography selection may be limited to peptide or nucleic acid libraries, while the other four approaches are applicable to peptide, non-peptide oligomer or small molecule libraries of substances (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of substances may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the substance screened by any of the present screening methods is converted by addition, deletion and/or replacement, is included in the substances obtained by the screening methods of the present invention.

Furthermore, when the screened test substance is a protein, for obtaining a DNA encoding the protein, either the whole amino acid sequence of the protein may be determined to deduce the nucleic acid sequence coding for the protein, or a partial amino acid sequence of the obtained protein may be analyzed to prepare an oligo DNA as a probe based on the sequence, and cDNA libraries may be screened with the probe to obtain a DNA encoding the protein. Alternatively, the DNA encoding the protein may be obtained by searching a database of protein or nucleic acid sequences such as Genbank. The obtained DNA may be confirmed by determining its usefulness in preparing the test substance which is a candidate for treating or preventing cancer.

Test substances useful in the screenings described herein can also be antibodies that specifically bind to an SUV420H1 or SUV420H2 protein or partial peptides thereof that lack the biological activity of the original proteins in vivo.

Although the construction of test substance libraries is well known in the art, herein below, additional guidance in identifying test substances and construction libraries of such substances for the present screening methods are provided.

In one aspect of the present invention, suppression of the expression level and/or biological activity of SUV420H1 or SUV420H2 leads to suppression of the growth of cancer cells. Therefore, when a substance suppresses the expression and/or activity of SUV420H1 or SUV420H2, such suppression is indicative of a potential therapeutic effect in a subject. In the context of the present invention, a potential therapeutic effect refers to a clinical benefit with a reasonable expectation. Examples of such clinical benefit include but are not limited to;

(a) reduction in expression of the SUV420H1 or SUV420H2 gene,

(b) a decrease in size, prevalence, growth, or metastatic potential of the cancer in the subject,

(c) preventing cancers from forming, or

(d) preventing or alleviating a clinical symptom of cancer.

(i) Molecular Modeling:

Construction of test substance libraries is facilitated by knowledge of the properties sought, and/or the molecular structure of SUV420H1 or SUV420H2 protein. One approach to preliminary screening of test substances suitable for further evaluation is computer modeling of the interaction between the test substance and SUV420H1 or SUV420H2 protein.

Computer modeling technology allows the visualization of the three-dimensional atomic structure of a selected molecule and the rational design of new substances that will interact with the molecule. The three-dimensional construct typically depends on data from x-ray crystallographic analysis or NMR imaging of the selected molecule. The molecular dynamics requires force field data. The computer graphics systems enable prediction of how a new substance will link to the target molecule and allow experimental manipulation of the structures of the substance and target molecule to perfect binding specificity. Prediction of what the molecule-substance interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMM and QUANTA programs, Polygen Corporation, Waltham, Mass. CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test substances” may be screened using the methods of the present invention to identify test substances for treating or preventing cancer, such as bladder cancer, cervical cancer, osteosarcoma, lung cancer, soft tissue tumor, breast cancer, chronic myelogenous leukemia (CML), esophageal cancer and gastric cancer.

(ii) Combinatorial Chemical Synthesis:

Combinatorial libraries of test substances may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(iii) Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 10⁶-10⁸ chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are exemplary. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists. Aptamers are macromolecules composed of nucleic acid that bind tightly to a specific molecular target. Tuerk and Gold (Science. 249:505-510 (1990)) discloses the SELEX (Systematic Evolution of Ligands by Exponential Enrichment) method for selection of aptamers. In the SELEX method, a large library of nucleic acid molecules (e.g., 10¹⁵ different molecules) can be used for screening.

Screening For a Substance Binding to SUV420H1 or SUV420H2 Polypeptide:

In the present invention, over-expression of SUV420H1 was detected in bladder cancer (FIGS. 1A and 1B), cervical cancer, osteosarcoma, and lung cancer (FIG. 2A), in spite of low expression in corresponding normal organs. Alternatively, over-expression of SUV420H2 was detected in bladder cancer (FIGS. 1A and 1B), breast cancer, chronic myelogenous leukemia (CML), esophageal cancer, gastric cancer, and lung cancer (FIG. 2B), in spite of low expression in corresponding normal organs.

Therefore, using the SUV420H1 or SUV420H2 gene or proteins encoded by the gene, the present invention provides a method of screening for a substance that binds to SUV420H1 or SUV420H2 polypeptide. Due to the expression of SUV420H1 or SUV420H2 in cancer such as bladder cancer, cervical cancer, osteosarcoma, lung cancer, soft tissue tumor, breast cancer, chronic myelogenous leukemia (CML), esophageal cancer and gastric cancer, substances that bind to SUV420H1 or SUV420H2 polypeptide may suppress the proliferation of cancer cells over-expressing either or both of SUV420H1 and SUV420H2 (e.g., bladder cancer cells, cervical cancer cells, osteosarcoma cells, lung cancer cells, soft tissue tumor cells, breast cancer cells, chronic myelogenous leukemia (CML) cells, esophageal cancer cells or gastric cancer cells), and thus be useful for treating or preventing cancer associating with either or both of SUV420H1 and SUV420H2 overexpression (e.g., bladder cancer, cervical cancer, osteosarcoma, lung cancer, soft tissue tumor, breast cancer, chronic myelogenous leukemia (CML), esophageal cancer or gastric cancer.). Therefore, the present invention also provides a method for screening for a substance that suppresses the proliferation of cancer cells overexpressing either or both of SUV420H1 and SUV420H2 (e.g., bladder cancer cells, cervical cancer cells, osteosarcoma cells, lung cancer cells, soft tissue tumor cells, breast cancer cells, chronic myelogenous leukemia (CML) cells, esophageal cancer cells and gastric cancer cells), and a method for screening for a substance for treating or preventing cancer associating with either or both of SUV420H1 and SUV420H2 overexpression (e.g., bladder cancer, cervical cancer, osteosarcoma, lung cancer, soft tissue tumor, breast cancer, chronic myelogenous leukemia (CML), esophageal cancer and gastric cancer) using the SUV420H1 or SUV420H2 polypeptide.

When SUV420H1 is targeted, the cancer may be selected from the group consisting of bladder cancer, cervical cancer, osteosarcoma, lung cancer and soft tissue tumor.

When SUV420H2 is targeted, the cancer may be selected from the group consisting of bladder cancer, breast cancer, chronic myelogenous leukemia (CML), esophageal cancer, gastric cancer and lung cancer. The lung cancer includes SCLC and NSCLC. Likewise, NSCLC includes adenocarcinoma, squamous cell carcinoma (SCC) and large-cell carcinoma.

Specifically, in an embodiment of the present method of screening for a candidate substance for treating or preventing cancer, or inhibiting cancer cell growth, the method may include the steps of:

(a) contacting a test substance with an SUV420H1 or SUV420H2 polypeptide or a fragment thereof;

(b) detecting the binding activity between the polypeptide or fragment thereof and the test substance; and

(c) selecting the test substance that binds to the polypeptide or a fragment as a candidate substance for treating or preventing cancer.

In another embodiment, the present invention may also provide a method of evaluating the therapeutic effect of a test substance on treating or preventing cancer, or inhibiting cancer cell growth, the method includes the steps;

(a) contacting a test substance with an SUV420H1 or SUV420H2 polypeptide or a fragment thereof;

(b) detecting the binding activity between the polypeptide or fragment thereof and the test substance; and

(c) correlating the potential therapeutic effect of the test substance with the binding activity detected in the step (b), wherein the potential therapeutic effect is shown when the test substance that binds to the polypeptide or a fragment is a candidate substance for treating or preventing cancer.

In the present invention, the therapeutic effect may be correlated with the binding activity to SUV420H1 or SUV420H2 polypeptide or a functional fragment thereof. For example, when the test substance binds to an SUV420H1 or SUV420H2 polypeptide or a functional fragment thereof, the test substance may identified or selected as the candidate substance having the therapeutic effect. Alternatively, when the test substance does not bind to SUV420H1 or SUV420H2 polypeptide or a functional fragment thereof, the test substance may identified as the substance having no significant therapeutic effect.

The method of the present invention will be described in more detail below.

The SUV420H1 or SUV420H2 polypeptide to be used for screening may be a recombinant polypeptide or a protein derived from the nature or a partial peptide thereof. The polypeptide to be contacted with a test substance can be, for example, a purified polypeptide, a soluble protein, a form bound to a carrier or a fusion protein fused with other polypeptides. In preferred embodiments, the polypeptide is isolated from cells expressing either or both of SUV420H1 and SUV420H2, or chemically synthesized to be contacted with a test substance in vitro.

As a method of screening for proteins, for example, that bind to the SUV420H1 or SUV420H2 polypeptide, many methods well known by a person skilled in the art can be used. Such a screening can be conducted by, for example, immunoprecipitation method, specifically, in the following manner. The gene encoding the SUV420H1 or SUV420H2 polypeptide may be expressed in a host (e.g., animal, bacterial, or fungal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and includes, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by the SUV420H1 or SUV420H2 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the SUV420H1 or SUV420H2 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the SUV420H1 or SUV420H2 polypeptide (Experimental Medicine 13: 85-90 (1995)).

In immunoprecipitation, an immune complex is formed by adding these antibodies to cell lysate prepared using an appropriate detergent. The immune complex consists of the SUV420H1 or SUV420H2 polypeptide, a polypeptide including the binding ability with the polypeptide, and an antibody. Immunoprecipitation can be also conducted using antibodies against the SUV420H1 or SUV420H2 polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by the SUV420H1 or SUV420H2 gene may be prepared as a fusion protein with an epitope, such as GST, an immune complex can then be formed in the same manner as in the use of the antibody against the SUV420H1 or SUV420H2 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the SUV420H1 or SUV420H2 polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cysteine, labeling proteins in the cells, and detecting the proteins. The target protein can be purified directly from the SDS-polyacrylamide gel and its sequence can be determined, when the molecular weight of a protein has been revealed.

As a method of screening for proteins binding to the SUV420H1 or SUV420H2 polypeptide, for example, West-Western blotting analysis (Skolnik et al., Cell 65: 83-90 (1991)) can be used. Specifically, a protein binding to the SUV420H1 or SUV420H2 polypeptide can be obtained by preparing a cDNA library from cultured cells (e.g., SW780, RT4, A549, LC319 and SBC5) expected to express a protein binding to the SUV420H1 or SUV420H2 polypeptide using a phage vector (e.g., ZAP), expressing the protein on LB-agarose, fixing the protein expressed on a filter, contacting the purified and labeled SUV420H1 or SUV420H2 polypeptide with the above filter, and detecting the plaques expressing proteins bound to the SUV420H1 or SUV420H2 polypeptide according to the label. The SUV420H1 or SUV420H2 polypeptide may be labeled by utilizing the binding between biotin and avidin, or by utilizing an antibody that specifically binds to the SUV420H1 or SUV420H2 or a peptide or polypeptide (for example, GST) that is fused to the SUV420H1 or SUV420H2 polypeptide. Methods using radioisotopes or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, SUV420H1 or SUV420H2 polypeptide is fused to the SRF-binding region or GAL4-binding region and expressed in yeast cells. A cDNA library is prepared from cells expected to express a protein binding to SUV420H1 or SUV420H2 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A substance binding to SUV420H1 or SUV420H2 polypeptide can also be screened using affinity chromatography. For example, SUV420H1 or SUV420H2 polypeptide may be immobilized on a carrier of an affinity column, and a test substance is applied to the column. test substances herein may be, for example, cell extracts, cell lysates, etc. After loading the test substance, the column is washed, and substances bound to SUV420H1 or SUV420H2 polypeptide can be prepared. When the test substance is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as means for detecting or quantifying the bound substance in the present invention. When such a biosensor is used, the interaction between SUV420H1 or SUV420H2 polypeptide and a test substance can be observed in real-time as a surface plasmon resonance signal, using only a minute amount of polypeptide without labeling (for example, BIAcore, Pharmacia). Therefore, it is possible to evaluate the binding between SUV420H1 or SUV420H2 polypeptide and a test substance using a biosensor such as BIAcore.

The methods of screening for substances that bind to SUV420H1 or SUV420H2 polypeptide when the immobilized SUV420H1 or SUV420H2 polypeptide is exposed to synthetic chemical substances, or natural substance banks or a random phage peptide display library, and the methods of screening using high-throughput combinatorial chemistry techniques (Wrighton et al., Science 273: 458-64 (1996); Verdine, Nature 384: 11-13 (1996); Hogan, Nature 384: 17-9 (1996)) to isolate not only proteins but chemical substances that bind to the SUV420H1 or SUV420H2 polypeptide (including agonist and antagonist) are well known to one skilled in the art.

In addition to the full length of SUV420H1 or SUV420H2 polypeptide, fragments of the polypeptides may be used for the present screening, so long as it retains at least one biological activity of the natural occurring SUV420H1 or SUV420H2 polypeptide. Such biological activities include cell proliferation activity, histone methyltransferase activity, anti-apoptotic activity and so on.

SUV420H1 or SUV420H2 polypeptides or fragments thereof may be further linked to other substances, so long as the polypeptides and fragments retain at least one of their biological activities. Usable substances include: peptides, lipids, sugar and sugar chains, acetyl groups, natural and synthetic polymers, etc. These kinds of modifications may be performed to confer additional functions or to stabilize the polypeptide and fragments.

SUV420H1 or SUV420H2 polypeptides or fragments thereof used for the present method may be obtained from nature as naturally occurring proteins via conventional purification methods or through chemical synthesis based on the selected amino acid sequence. For example, conventional peptide synthesis methods that can be adopted for the synthesis include:
1) Peptide Synthesis, Interscience, New York, 1966;
2) The Proteins, Vol. 2, Academic Press, New York, 1976;
3) Peptide Synthesis (in Japanese), Maruzen Co., 1975;
4) Basics and Experiment of Peptide Synthesis (in Japanese), Maruzen Co., 1985;
5) Development of Pharmaceuticals (second volume) (in Japanese), Vol. 14 (peptide synthesis), Hirokawa, 1991;
6) WO99/67288; and
7) Barany G. & Merrifield R. B., Peptides Vol. 2, “Solid Phase Peptide Synthesis”, Academic Press, New York, 1980, 100-118.

Alternatively, SUV420H1 or SUV420H2 polypeptides may be obtained through any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding the SUV420H1 or SUV420H2 polypeptide is expressed in host (e.g., animal) cells and such by inserting the gene into a vector for expressing foreign genes, such as pSV2neo, pcDNA I, pcDNA3.1, pCAGGS, or pCD8. A promoter may be used for the expression. Any commonly used promoters may be employed including, for example, the SV40 early promoter (Rigby in Williamson (ed.), Genetic Engineering, vol. 3. Academic Press, London, 1982, 83-141), the EF-alpha promoter (Kim et al., Gene 1990, 91:217-23), the CAG promoter (Niwa et al., Gene 1991, 108:193), the RSV LTR promoter (Cullen, Methods in Enzymology 1987, 152:684-704), the SR alpha promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet. 1982, 1:385-94), the Adenovirus late promoter (Kaufman et al., Mol Cell Biol 1989, 9:946), the HSV TK promoter, and such. The introduction of the vector into host cells to express the SUV420H1 or SUV420H2 gene can be performed according to any methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1985, 4:1641-3), the Lipofectin method (Derijard B, Cell 1994, 7:1025-37; Lamb et al., Nature Genetics 1993, 5:22-30; Rabindran et al., Science 1993, 259:230-4), and such.

The SUV420H1 or SUV420H2 polypeptide may also be produced in an in vitro transcription and in vitro translation system.

The SUV420H1 or SUV420H2 polypeptide to be contacted with a test substance can be, for example, a purified polypeptide, a soluble protein, or a fusion protein fused with other polypeptides.

Test substances screened by the present method as substances that bind to SUV420H1 or SUV420H2 polypeptide can be candidate substances that have the potential to treat or prevent cancers. The potential of these candidate substances to treat or prevent cancers may be evaluated by second and/or further screening to identify therapeutic substance for cancers. For example, these candidate substances may further examined their ability of suppressing cancer cell proliferation by being contacted with a cancer cell overexpressing SUV420H1 or SUV420H2 gene.

Screening for a Substance Suppressing the Biological Activity of SUV420H1 or SUV420H2 Polypeptide:

The present invention provides a method for screening for a substance that suppresses a biological activity of SUV420H1 or SUV420H2 polypeptide (e.g., cancer cell proliferation enhancing activity), and a method for screening for a candidate substance for treating or preventing cancer associating with either or both of SUV420H1 and SUV420H2 overexpression, including bladder cancer, cervical cancer, osteosarcoma, lung cancer, soft tissue tumor, breast cancer, chronic myelogenous leukemia (CML), esophageal cancer and gastric cancer. When SUV420H1 is targeted, the cancer may be selected from the group consisting of bladder cancer, cervical cancer, osteosarcoma, lung cancer and soft tissue tumor. When SUV420H2 is targeted, the cancer may be selected from the group consisting of bladder cancer, breast cancer, chronic myelogenous leukemia (CML), esophageal cancer, gastric cancer and lung cancer. The lung cancer includes SCLC and NSCLC. Likewise, NSCLC includes adenocarcinoma, squamous cell carcinoma (SCC) and large-cell carcinoma.

Thus, the present invention provides a method of screening for a candidate substance for treating or preventing cancer, or inhibiting cancer cell growth, including the steps as follows:

(a) contacting a test substance with an SUV420H1 or SUV420H2 polypeptide or a fragment thereof;

(b) detecting the biological activity of the polypeptide of step (a); and

(c) selecting the test substance that suppresses the biological activity of the polypeptide as compared to the biological activity of the polypeptide detected in the absence of the test substance.

In another embodiment, the present invention provides a method of evaluating therapeutic effect of a test substance on treating or preventing cancer, or inhibiting cancer cell growth, the method including the steps as follows:

(a) contacting a test substance with an SUV420H1 or SUV420H2 polypeptide or a fragment thereof;

(b) detecting the biological activity of the polypeptide of step (a); and

(c) correlating the potential therapeutic effect of the test substance with the biological activity detected in step (b), wherein the potential therapeutic effect is shown, when the test substance suppresses the biological activity of the polypeptide as compared to the biological activity of the polypeptide detected in the absence of the test substance.

Alternatively, in some embodiments, the present invention provides a method for evaluating or estimating a therapeutic effect of a test substance on treating or preventing cancer or inhibiting cancer associated with over-expression of either or both of SUV420H1 and SUV420H2, the method including steps of:

(a) contacting a test substance with a polypeptide encoded by a polynucleotide of SUV420H1 or SUV420H2 gene, or fragment thereof;
(b) detecting the biological activity of the polypeptide of step (a); and
(c) correlating the potential therapeutic effect and the test substance, wherein the potential therapeutic effect is shown, when the test substance suppresses the biological activity of the polypeptide encoded by the polynucleotide of SUV420H1 or SUV420H2 gene as compared to the biological activity of said polypeptide detected in the absence of the test substance.

Such cancer includes bladder cancer, cervical cancer, osteosarcoma, lung cancer, soft tissue tumor, breast cancer, chronic myelogenous leukemia (CML), esophageal cancer and gastric cancer. In the present invention, the therapeutic effect may be correlated with the biological activity of SUV420H1 or SUV420H2 polypeptide. For example, when the test substance suppresses or inhibits the biological activity of SUV420H1 or SUV420H2 polypeptide as compared to a level detected in the absence of the substance, the test substance may identified or selected as a candidate substance having the therapeutic effect. Alternatively, when the test substance does not suppress or inhibit the biological activity of SUV420H1 or SUV420H2 polypeptide as compared to a level detected in the absence of the substance, the test substance may identified as a substance having no significant therapeutic effect.

The method of the present invention will be described in more detail below.

Any polypeptides can be used for screening so long as they retain a biological activity of the SUV420H1 or SUV420H2 polypeptide. For example, SUV420H1 or SUV420H2 polypeptide and functionally equivalent thereof can be used in the present screening method. Examples of biological activities of SUV420H1 or SUV420H2 polypeptide include cell proliferation enhancing activity, methyltransferase activity, and anti-apoptotic activity. These activities may be used as indexes for the screening.

The substance isolated by this screening is a candidate for antagonists of the SUV420H1 or SUV420H2 polypeptide. The term “antagonist” refers to molecules that inhibit the function of the polypeptide by binding thereto. The term also refers to molecules that reduce or inhibit expression of the gene encoding SUV420H1 or SUV420H2. Moreover, a substance isolated by this screening is a candidate for substances which inhibit the in vivo interaction of the SUV420H1 or SUV420H2 polypeptide with other molecules (including DNAs and proteins).

In the present invention, suppressing the expression of SUV420H1 or SUV420H2 gene reduces cell growth. Thus, by screening for a candidate substance that reduces the biological activity of SUV420H1 or SUV420H2 polypeptide, a candidate substance that has the potential to treat or prevent cancers can be identified. The potential of these candidate substances to treat or prevent cancers may be evaluated by second and/or further screening to identify therapeutic substance for cancers e.g. bladder cancer, cervical cancer, osteosarcoma, lung cancer, soft tissue tumor, breast cancer, chronic myelogenous leukemia (CML), esophageal cancer and gastric cancer.

When the biological activity to be detected in the present method is cell proliferation enhancing activity, it can be detected, for example, by preparing cells which express the SUV420H1 or SUV420H2 polypeptide, culturing the cells in the presence of a test substance, and determining the speed of cell proliferation, measuring the cell cycle and such, as well as by measuring survival of cells or colony forming activity, for example, as shown in FIGS. 3 and 4. The substances that reduce the speed of proliferation of the cells expressing SUV420H1 or SUV420H2 may be selected as candidate substances for treating or preventing cancer, particularly cancers including bladder cancer, cervical cancer, osteosarcoma, lung cancer, soft tissue tumor, breast cancer, chronic myelogenous leukemia (CML), esophageal cancer and gastric cancer. In some embodiments, cells expressing SUV420H1 or SUV420H2 gene may be isolated cells or cultured cells, which exogenously or endogenously express SUV420H1 or SUV420H2 gene in vitro.

More specifically, the method may include the step of:

(a) contacting a test substance with cells overexpressing SUV420H1 or SUV420H2;

(b) measuring cell proliferation enhancing activity in the cells of step (a); and

(c) selecting the test substance that reduces the cell proliferation enhancing activity in the comparison with the cell proliferation enhancing activity in the absence of the test substance.

In some embodiments, the method of the present invention may further include the step of:

(d) selecting the test substance that has no effect on the cells expressing little or no SUV420H1 or SUV420H2.

When the biological activity to be detected in the present method is methyltransferase activity, the methyltransferase activity can be determined by contacting a polypeptide with a substrate (e.g., histone H4K20,H3K9) and a co-factor (e.g., S-adenosyl-L-methionine) under conditions suitable for methylation of the substrate and detecting the methylation level of the substrate.

More specifically, the may method include the step of:

(a) contacting an SUV420H1 or SUV420H2 polypeptide or fragment thereof and a substrate for methylation in the presence of a test substance;

(b) detecting the methylation level of the substrate; and

(c) selecting the test substance that suppresses the methylation level of the substrate as compared to that detected in the absence of the test substance.

In the present invention, methyltransferase activity of a SUV420H1 or SUV420H2 polypeptide can be determined by methods known in the art. For example, the SUV420H1 or SUV420H2 and a substrate can be incubated with a labeled methyl donor, under a suitable methylation assay condition. A histone (e.g., histone H4) peptide (full length of a histone or fragment thereof) and S-adenosyl-L-methionine (SAM) can be used as a substrate and methyl donor, respectively. In typical embodiments, histone H4 fragments that includes the 20th lysine of histone H4 and has 10 or more amino acid residues, 15 or more amino acid residues, 20 or more amino acid residues, 25 or more amino acid residues, 30 or more amino acid residues, or 35 or more amino acid residues may be used as substrates. Transfer of the radiolabel to the histone peptides can be detected, for example, by SDS-PAGE electrophoresis and fluorography. Alternatively, following the reaction the histone peptides can be separated from the methyl donor by filtration, and the amount of radiolabel retained on the filter quantitated by scintillation counting. Other suitable labels that can be attached to methyl donors, such as chromogenic and fluorescent labels, and methods of detecting transfer of these labels to histone peptides, are known in the art.

Alternatively, the methyltransferase activity of SUV420H1 or SUV420H2 polypeptide can be determined using an unlabeled methyl donor and reagents that selectively recognize methylated histone peptides. For example, after incubation of the SUV420H1 or SUV420H2 polypeptide, a substrate (e.g., histone H4 or fragment thereof) to be methylated and methyl donor, under a condition capable of methylation of the substrate, the level of the methylated substrate can be detected by immunological method. Any immunological techniques using an antibody recognizing methylated substrate can be used for the detection. For example, an antibody against methylated histone (e.g. histone H4K20me2 or histone H4K20me3) is commercially available (abcam Ltd.). ELISA or Immunoblotting with antibodies recognizing methylated histone can be used for the present invention.

Furthermore, the present method detecting methyltransferase activity can be performed by preparing cells which express the SUV420H1 or SUV420H2 polypeptide, culturing the cells in the presence of a test substance, and determining methylation level of a histone (especially, histone H4K20), for example, by using the antibody specific binding to methylation region.

More specifically, the method may include the step of:

[1] contacting a test substance with cells expressing SUV420H1 or SUV420H2;

[2] detecting a methylation level of histone; and

[3] selecting the test substance that reduces the methylation level of the histone in comparison with the methylation level in the absence of the test substance.

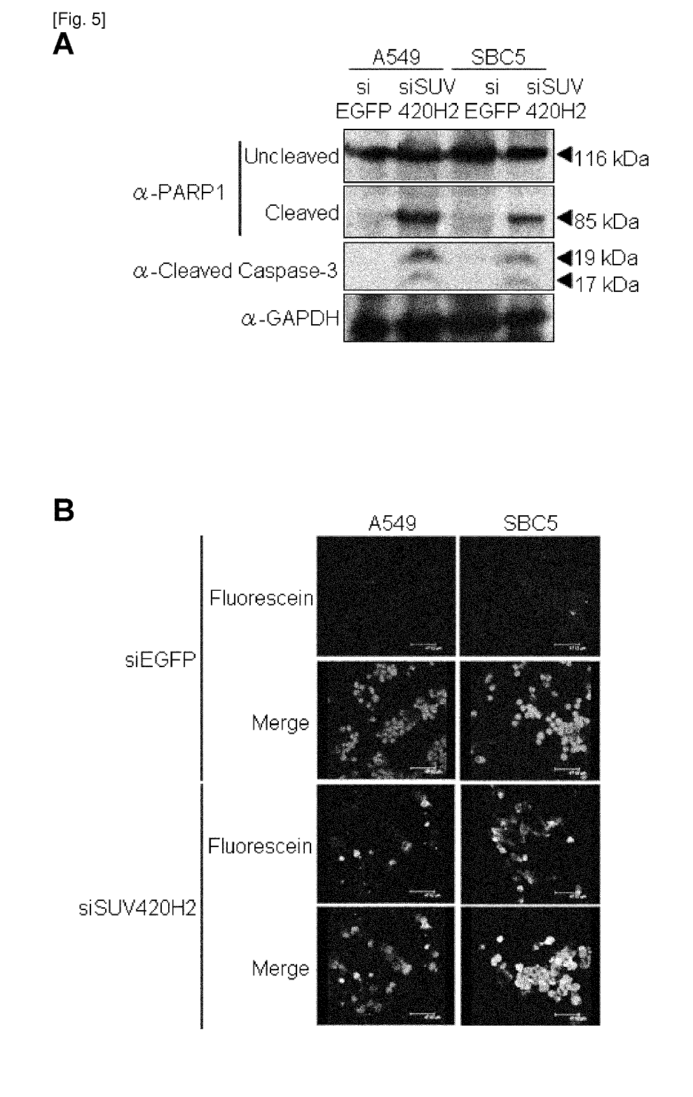

In the present invention, suppressing the expression of SUV420H2 gene induces apoptosis of cancer cells (FIGS. 5A and 5B). When the biological activity to be detected in the present method is anti-apoptotic activity, it can be detected, for example, by preparing cells which express the SUV420H1 or SUV420H2 polypeptide, culturing the cells in the presence of a test substance, and detecting the cleavage of PARP1 and/or Caspase 3, which is an marker of apoptosis induction, or DNA fragmentation by TUNEL assay, for example, as shown in FIGS. 5A and 5B. The substances that induce apoptosis of the cells expressing SUV420H1 or SUV420H2 may be selected as candidate substances for treating or preventing cancer, particularly cancers including bladder cancer, cervical cancer, osteosarcoma, lung cancer, soft tissue tumor, breast cancer, chronic myelogenous leukemia (CML), esophageal cancer and gastric cancer. In some embodiments, cells expressing SUV420H1 or SUV420H2 gene may be isolated cells or cultured cells, which exogenously or endogenously express SUV420H1 or SUV420H2 gene in vitro.

More specifically, the method may include the step of:

(a) contacting a test substance with cells overexpressing SUV420H1 or SUV420H2;

(b) detecting apoptosis level of cells of step (a); and

(c) selecting the test substance that increases the apoptosis level in the comparison with the apoptosis level in the absence of the test substance.

In some embodiments, the method of the present invention may further include the step of:

(d) selecting the test substance that has no effect on the cells expressing little or no SUV420H1 or SUV420H2.

Cells expressing either or both of SUV420H1 and SUV420H2 polypeptides include, for example, cell lines established from cancer, e.g. bladder cancer, cervical cancer, osteosarcoma, lung cancer, soft tissue tumor, breast cancer, chronic myelogenous leukemia (CML), esophageal cancer or gastric cancer, such cells can be used for the above screening of the present invention so long as the cells express the gene. Alternatively cells can be transfected an expression vectors of SUV420H1 or SUV420H2 polypeptide, so as to express the gene.

“Suppress the biological activity (e.g., cell proliferation enhancing activity, methyltransferase activity or anti-apoptotic activity)” as defined herein may be at least 10% suppression in comparison with in absence of the substance, at least 25%, 50% or 75% suppression, or at least at 90% suppression.

In some embodiments, control cells which do not express SUV420H1 or SUV420H2 polypeptide are used. Accordingly, the present invention also provides a method of screening for a candidate substance for inhibiting the cell growth or a candidate substance

for treating or preventing SUV420H1 or SUV420H2 associating disease, using the SUV420H1 or SUV420H2 polypeptide or fragments thereof including the steps as follows:

a) culturing cells which express an SUV420H1 or SUV420H2 polypeptide or a functional fragment thereof, and control cells that do not express an SUV420H1 or SUV420H2polypeptide or a functional fragment thereof in the presence of a test substance;
b) detecting the biological activity of the cells which express the protein and control cells; and
c) selecting the test substance that inhibits the biological activity in the cells which express the protein as compared to the biological activity detected in the control cells and in the absence of said test substance.

Furthermore, the present invention also provides a method for screening a candidate substance for inhibiting or reducing a cancer cell growth, which cancer cell expresses SUV420H1 or SUV420H2, e.g. bladder cancer, cervical cancer, osteosarcoma, lung cancer, soft tissue tumor, breast cancer, chronic myelogenous leukemia (CML), esophageal cancer or gastric cancer cell, and screening a candidate substance for treating or preventing cancer associated with SUV420H1 or SUV420H2 overexpression, e.g. bladder cancer, cervical cancer, osteosarcoma, lung cancer, soft tissue tumor, breast cancer, chronic myelogenous leukemia (CML), esophageal cancer or gastric cancer.

Screening For a Substance Altering the Expression of SUV420H1 or SUV420H2:

The present invention provides a method of screening for a substance that inhibits the expression of SUV420H1 or SUV420H2. A substance that inhibits the expression of SUV420H1 or SUV420H2 is expected to suppress the proliferation of cancer cells (e.g., bladder cancer, cervical cancer, osteosarcoma, lung cancer, soft tissue tumor, breast cancer, chronic myelogenous leukemia, esophageal cancer or gastric cancer cells), and thus may be useful for treating or preventing cancer (e.g., bladder cancer, cervical cancer, osteosarcoma, lung cancer, soft tissue tumor, breast cancer, chronic myelogenous leukemia, esophageal cancer or gastric cancer). Therefore, the present invention also provides a method for screening a substance that suppresses the proliferation of cancer cells overexpressing SUV420H1 or SUV420H2, such as bladder cancer, cervical cancer, osteosarcoma, lung cancer, soft tissue tumor, breast cancer, chronic myelogenous leukemia, esophageal cancer and gastric cancer cells, and a method for screening a candidate substance for treating or preventing cancer associating with SUV420H1 or SUV420H2 overexpression such as bladder cancer, cervical cancer, osteosarcoma, lung cancer, soft tissue tumor, breast cancer, chronic myelogenous leukemia, esophageal cancer or gastric cancer. In some embodiments, the cancer associating with SUV420H1 includes bladder cancer, cervical cancer, osteosarcoma, lung cancer, and soft tissue tumor, the cancer associating with SUV420H2 includes bladder cancer, breast cancer, chronic myelogenous leukemia, esophageal cancer, gastric cancer and lung cancer.

When SUV420H1 is targeted, the cancer may be selected from the group consisting of bladder cancer, cervical cancer, osteosarcoma, lung cancer and soft tissue tumor. When SUV420H2 is targeted, the cancer may be selected from the group consisting of bladder cancer, breast cancer, chronic myelogenous leukemia (CML), esophageal cancer, gastric cancer and lung cancer. The lung cancer includes SCLC and NSCLC. Likewise, “NSCLC” includes adenocarcinoma, squamous cell carcinoma (SCC) and large-cell carcinoma.

In the context of the present invention, such screening may include, for example, the following steps:

(a) contacting a test substance with a cell expressing SUV420H1 or SUV420H2 gene;

(b) detecting the expression level of SUV420H1 or SUV420H2 gene in the cell; and

(c) selecting the test substance that reduces the expression level of SUV420H1 or SUV420H2 gene in comparison with the expression level detected in absence of the test substance.

Furthermore, the present invention provides a method of evaluating the therapeutic effect of a test substance on suppressing the proliferation of cancer cells or treating or preventing cancer, the method may include steps of:

(a) contacting a substance with a cell expressing the SUV420H1 or SUV420H2 gene;

(b) detecting the expression level of the SUV420H1 or SUV420H2 gene; and

(c) correlating the potential therapeutic effect of the test substance with the expression level detected in step (b), wherein the potential therapeutic effect is shown when the test substance reduces the expression level of SUV420H1 or SUV420H2 gene in comparison with the expression level detected in absence of the test substance.

In the present invention, the therapeutic effect may be correlated with the expression level of the SUV420H1 or SUV420H2 gene. For example, when the test substance reduces the expression level of the SUV420H1 or SUV420H2 gene as compared to a level detected in the absence of the test substance, the test substance may identified or selected as the candidate substance having the therapeutic effect. Alternatively, when the test substance does not reduce the expression level of the SUV420H1 or SUV420H2 gene as compared to a level detected in the absence of the test substance, the test substance may identified as a substance having no significant therapeutic effect.

The method of the present invention will be described in more detail below.

Cells expressing the SUV420H1 or SUV420H2 gene include, for example, cell lines established from bladder cancer, cervical cancer, osteosarcoma, lung cancer, e.g. SCLC, soft tissue tumor, breast cancer, chronic myelogenous leukemia, esophageal cancer or gastric cancer; such cells can be used for the above screening methods of the present invention (e.g., SW780, RT4, A549, LC319, SBC5). The expression level can be estimated by methods well known to one skilled in the art, for example, RT-PCR, Northern blot assay, Western blot assay, immunostaining and flow cytometry analysis. “Reduce the expression level” as defined herein may be at least 10% reduction of expression level of SUV420H1 or SUV420H2 gene in comparison to the expression level in absence of the test substance, at least 25%, 50% or 75% reduced level, or at least 95% reduced level. Test substances herein include, for example, chemical substances, double-strand molecules, and so on. Methods for preparation of chemical substances and the double-strand molecules are described in the above description. In the method of screening, test substances that reduce the expression level of the SUV420H1 or SUV420H2 gene can be selected as candidate substances to be used for the treatment or prevention of cancer associating SUV420H1 or SUV420H2 overexpression, such as bladder cancer, cervical cancer, osteosarcoma, lung cancer, e.g. SCLC, soft tissue tumor, breast cancer, chronic myelogenous leukemia, esophageal cancer and gastric cancer. In some embodiments, cells expressing SUV420H1 or SUV420H2 gene may be isolated cells or cultured cells, which exogenously or endogenously express SUV420H1 or SUV420H2 gene in vitro.

The potential of these candidate substances to treat or prevent cancers may be evaluated by second and/or further screening to identify therapeutic substance for cancers.

Alternatively, the screening method of the present invention may include the following steps:

(a) contacting a test substance with a cell into which a vector, including the transcriptional regulatory region of SUV420H1 or SUV420H2 gene, and a reporter gene that is expressed under the control of the transcriptional regulatory region, has been introduced;

(b) detecting the expression level or activity of the reporter gene; and

(c) selecting the test substance that reduces the expression level or activity of the reporter gene. in comparison with the expression level or activity detected in absence of the test substance.

Furthermore, the present invention provides a method of evaluating therapeutic effect of a test substance on treating or preventing cancer or inhibiting cancer cell growth, the method including steps of:

(a) contacting a test substance with a cell into which a vector, including the transcriptional regulatory region of SUV420H1 or SUV420H2 gene, and a reporter gene that is expressed under the control of the transcriptional regulatory region, has been introduced;

(b) detecting the expression level or activity of the reporter gene; and

(c) correlating the potential therapeutic effect of the test substance with the expression level or activity detected in step (b), wherein the potential therapeutic effect is shown, when the test substance reduces the expression level or activity of the reporter gene in comparison with the expression level or activity detected in absence of the test substance.

In the present invention, the therapeutic effect may be correlated with the expression level or activity of the reporter gene. For example, when the test substance reduces the expression level or activity of the reporter gene as compared to a level detected in the absence of the test substance, the test substance may identified or selected as the candidate substance having the therapeutic effect. Alternatively, when the test substance does not reduce the expression level or activity of said reporter gene as compared to a level detected in the absence of the test substance, the test substance may identified as the substance having no significant therapeutic effect.

Suitable reporter genes and host cells are well known in the art. For example, reporter genes include luciferase, green florescence protein (GFP), Discosoma sp. Red Fluorescent Protein (DsRed), Chrolamphenicol Acetyltransferase (CAT), lacZ and beta-glucuronidase (GUS), and host cells include COS7, HEK293, HeLa and so on. The reporter construct required for the screening can be prepared by connecting reporter gene sequence to the transcriptional regulatory region of SUV420H1 or SUV420H2. The transcriptional regulatory region of SUV420H1 or SUV420H2 herein is the region from start codon to at least 500 bp upstream, such as at least 1,000 bp, 5000 bp, or 10,000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the reporter construct may be introduced into host cells and the expression or activity of the reporter gene may be detected by methods well known in the art (e.g., using luminometer, absorption spectrometer, flow cytometer and so on). “Reduces the expression or activity” as defined herein means at least 10% reduction of the expression or activity of the reporter gene in comparison with in absence of the substance, least 25%, 50% or 75% reduction, or at least 95% reduction.

In the present invention, suppressing the expression of SUV420H1 or SUV420H2 gene reduces cell growth. Thus, by screening for a candidate substance that reduces the expression or activity of the reporter gene, a candidate substance that has the potential to treat or prevent cancers can be identified. The potential of these candidate substances to treat or prevent cancers may be evaluated by second and/or further screening to identify therapeutic substance for cancers. For example, when a substance that binds to SUV420H1 or SUV420H2 polypeptide also inhibits an activity of the cancer, it may be concluded that such substance has SUV420H1 or SUV420H2 specific therapeutic effect. For example, in the context of the present invention, such activity includes cancer cell growth and metastatic activity of cancer.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### EXAMPLES

The invention will be further described in the following examples, which do not limit the scope of the invention described in the claims.

### Example 1

### Materials and Methods

Tissue samples and RNA preparation. 124 surgical specimens of primary urothelial carcinoma were collected, either at cystectomy (including total/simple/partial) or transurethral resection of bladder tumor (TUR-Bt), and snap frozen in liquid nitrogen. 28 specimens of normal bladder urothelium were collected from areas of macroscopically normal bladder urothelium in patients with no evidence of malignancy. Use of tissues for this study was approved by Cambridge shire Local Research Ethics Committee. A total of 30 sections of 30 micrometers were homogenized for RNA extraction and two 7 micrometer ‘sandwich’ sections adjacent to the tissue used for RNA extraction were sectioned, stained and assessed for cellularity and tumor grade by an independent consultant urohistopathologist. Additionally, the sections were graded according to the degree of inflammatory cell infiltration (low, moderate and severe). Samples showing significant inflammatory cell infiltration were excluded [Wallard M J, et al., British journal of cancer 2006; 94: 569-577].

Total RNA was extracted using TRI Reagent™ (Sigma, Dorset, UK), following the manufacturers' protocol. RNeasy Minikits™ (QIAGEN, Crawley, UK), including a DNase step, were used to optimize RNA purity. Agilent 2100™ total RNA bioanalysis was performed. 1 microliter of resuspended RNA from each sample was applied to an RNA 6000 Nano Lab Chip™ and processed according to the manufacturers' instructions. All chips and reagents were sourced from Agilent Technologies™ (West Lothian, UK).

Reverse transcription. Total RNA concentrations were determined using the NanoDrop™ ND 1000 spectrophotometer (Nyxor Biotech, Paris, France). 1 microgram of total RNA was reverse transcribed with 2 microgram random hexamers (Amersham) and Superscript III reverse transcriptase (Invitrogen, Paisley, UK) in 20 microliter reactions according to the manufacturer's instructions. cDNA was then diluted 1:100 with PCR grade water and stored at −20 degrees C.

Laser capture microdissection. Tissue for laser capture microdissection was collected prospectively following the procedure outlined above. Five sequential sections of 7 micrometer thickness were cut from each tissue and stained using Histogene™ staining solution (Arcturus, Calif., USA) following the manufacturer's protocol. Slides were then immediately transferred for microdissection using a Pix Cell II laser capture microscope (Arcturus, Calif., USA). This technique employs a low-power infrared laser to melt a thermoplastic film over the cells of interest, to which the cells become attached.

Approximately 10,000 cells were microdissected from both stromal and epithelial/cancerous compartments in each tissue. RNA was extracted using an RNeasy Micro Kit (QIAGEN, Crawley, UK). Areas of cancer or stroma containing significant inflammatory areas of tumor or stroma containing significant inflammatory cell infiltration were avoided to prevent contamination.

Total RNA was reverse transcribed and qRT-PCR performed as above. Given the low yield of RNA from such small samples, NanoDrop™ quantification was not performed, but correction for the endogenous 18S CT value was used as an accurate measure of the amount of intact starting RNA. Transcript analysis was performed for the SUV420H1 and SUV420H2 genes.

To validate the accuracy of microdissection, primers and probes for Vimentin and Uroplakin were sourced and qRT-PCR performed according to the manufacturer's instructions (Assays on demand, Applied Biosystems, Warrington, UK). Vimentin is primarily expressed in mesenchymally derived cells, and was used as a stromal marker. Uroplakin is a marker of urothelial differentiation and is preserved in up to 90% of epithelially derived tumors [Olsburgh J, et al., The Journal of pathology 2003; 199:41-49211.

Cell culture. All cell lines were grown in monolayers in appropriate media: Eagle's minimal essential medium (EMEM) for 253J, 253]-BV, HT1197, HT1376, J82, SCaBER, UMUC3 bladder cancer cells and SBC5 small cell lung cancer cells; RPMI1640 medium for 5637 bladder cancer cells and A549, H2170 and LC319 non-small cell lung cancer cells; Dulbecco's modified Eagle's medium (DMEM) for EJ28 bladder cancer cells and RERF-LC-AI non-small cell lung cancer cells; McCoy's 5A medium for RT4 and T24 bladder cancer cells; Leibovitz's L-15 for SW780 cells supplemented with 10% fetal bovine serum and 1% antibiotic/antimycotic solution (Sigma). All cells were maintained at 37 degrees C. in humid air with 5% CO₂, (253J, 253J-BV, HT1197, HT1376, J82, SCaBER, UMUC3, SBC5, 5637, A549 H2170, LC319, EJ28, RERF-LC-AI, RT4 and T24) or without CO₂ (SW780). Cells were transfected with FuGENE6 (ROCHE, Basel, Switzerland) according to manufacturers' protocols.

Quantitative Real-time PCR. As described previously, 124 bladder cancer and normal 28 bladder tissues were prepared in Cambridge Addenbrooke's Hospital. For quantitative RT-PCR reactions, specific primers for all human GAPDH (housekeeping gene), SDH (housekeeping gene), SUV420H1 and SUV420H2 were designed (primer sequences in Table 1). PCR reactions were performed using the ABI prism 7700 Sequence Detection System (Applied Biosystems, Warrington, UK) following the manufacturer's protocol. 50% SYBR GREEN universal PCR Master Mix without UNG (Applied Biosystems, Warrington, UK), 50 nM each of the forward and reverse primers and 2 microliter of reverse transcriptional cDNA were applied. Amplification conditions were firstly 5 min at 95 degrees C. and then 45 cycles each consisting of 10 sec at 95 degrees C., 1 min at 55 degrees C. and 10 sec at 72 degrees C. After this, the condition was set for 15 sec at 95 degrees C., 1 min at 65 degrees C. to draw the melting curve, and cool to 50 degrees C. for 10 sec. Reaction conditions for target gene amplification were as described above and the equivalent of 5 ng of reverse transcribed RNA was used in each reaction.

To determine relative RNA levels within the samples, standard curves for the PCR reactions were prepared from a series of two-fold dilutions of cDNA covering the range 2-0.625 ng of RNA for the 18S reaction and 20-0.5 ng of RNA for all target genes. The ABI prism 7700 measured changes in fluorescence levels throughout the 45 cycles PCR reaction and generated a cycle threshold (Cₜ) value for each sample correlating to the point at which amplification entered the exponential phase. This value was used as an indicator of the amount of starting template; hence a lower Cₜ values indicated a higher amount of initial intact cDNA.

TABLE 1
Primer sequences for quantitative RT-PCR.

| Gene name | Primer sequence |
|---|---|
| GAPDH (housekeeping gene)-f | 5′ GCAAATTCCATGGCACCGTC 3′ |
| | (SEQ ID NO: 1) |
| GAPDH (housekeeping gene)-r | 5′ TCGCCCCACTTGATTTTGG 3′ |
| | (SEQ ID NO: 2) |
| SDH (housekeeping gene)-f | 5′ TGGGAACAAGAGGGCATCTG 3′ |
| | (SEQ ID NO: 3) |
| SDH (housekeeping gene)-r | 5′ CCACCACTGCATCAAATTCATG 3′ |
| | (SEQ ID NO: 4) |
| SUV420H1-f1 | 5′ ATGTCAAGAATTCCAGCTTCTTCC 3′ |
| | (SEQ ID NO: 5) |
| SUV420H1-r1 | 5′ CAATTTTATCTTTGAAAGTAAAGG 3′ |
| | (SEQ ID NO: 6) |
| SUV420H1-f2 | 5′ AGAAATCATTGCAAGCGGCTGGAG 3′ |
| | (SEQ ID NO: 7) |
| SUV420H1-r2 | 5′ CTGGCTCCTTATCTTTTTTAATGG 3′ |
| | (SEQ TD NO: 8) |
| SUV420H2-f1 | 5′ TATGGGCTGCCTTACGTGGTGCGTG 3′ |
| | (SEQ ID NO: 9) |
| SUV420H2-r1 | 5′ CGGGATCAGGATGGGGCCTGGGGTC 3′ |
| | (SEQ ID NO: 10) |
| SUV420H2-f2 | 5′ GCAGGCCCTCGCCTTCGCCCCCTTC 3′ |
| | (SEQ ID NO: 11) |
| SUV420H2-r2 | 5′ TCCGGCCTGTCACAGCTCTTCACC 3′ |
| | (SEQ ID NO: 12) |

Transfection with siRNAs. The siRNA oligonucleotide duplexes were purchased from SIGMA Genosys for targeting the human SUV420H1/H2 transcripts or the EGFP transcript as control siRNAs. The siRNA sequences are described in Table 2. siRNA duplexes (100 nM final concentration) were transfected in bladder and lung cancer cell lines with Lipofectamine 2000 (Invitrogen) for 48 hours, and checked the cell viability using cell counting kit 8 (DOJINDO).

TABLE 2
siRNA target sequences.

| siRNA name | | Sequence |
|---|---|---|
| siEGFP | | Sense: 5′ GCAGCACGACUUCUUCAAGTT 3′ (SEQ ID NO: 13) |
| | | Antisense: 5′ CUUGAAGAAGUCGUGCUGCTT 3′ (SEQ ID NO: 14) |
| | | Target: 5′ GCAGCACGACUUCUUCAAG 3′ (SEQ ID NO: 33) |
| siFFLuc | | Sense: 5′ GUGCGCUGCUGGUGCCAACTT 3′ (SEQ ID NO: 34) |
| | | Antisense: 5′ CUUGAAGAAGUCGUGCUGCTT 3′(SEQ ID NO: 35) |
| | | Target: 5′ GUGCGCUGCUGGUGCCAAC 3′(SEQ ID NO: 36) |
| siNegative | Target#1 | Sense: 5′ AUCCGCGCGAUAGUACGUA 3′ (SEQ ID NO: 37) |
| control | | Antisense: 5′ UACGUACUAUCGCGCGGAU 3′ (SEQ ID NO: 38) |
| (Cocktail) | | Target: 5′ AUCCGCGCGAUAGUACGUA 3′ (SEQ ID NO: 39) |
| | Target#2 | Sense: 5′ UUACGCGUAGCGUAAUACG 3′ (SEQ ID NO: 40) |
| | | Antisense: 5′ CGUAUUACGCUACGCGUAA 3′ (SEQ ID NO: 41) |
| | | Target: 5′ UUACGCGUAGCGUAAUACG 3′ (SEQ ID NO: 42) |
| | Target#3 | Sense: 5′ UAUUCGCGCGUAUAGCGGU 3′ (SEQ ID NO: 43) |
| | | Antisense: 5′ ACCGCUAUACGCGCGAAUA 3′ (SEQ ID NO:44) |
| | | Target: 5′ UAUUCGCGCGUAUAGCGGU 3′ (SEQ ID NO: 45) |

| | |
|---|---|
| siSUV420H1#1 | Sense: 5′ GAGUUCUGCGAGUGUUACATT 3′ (SEQ ID NO: 15) |
| | Antisense: 5′ UGUAACACUCGCAGAACUCTT 3′ (SEQ ID NO: 16) |
| | Target: 5′ GAGUUCUGCGAGUGUUACA 3′ (SEQ ID NO: 29) |
| siSUV420H1#2 | Sense: 5′ GAAAUUAUUCAAAGAACAUTT 3′ (SEQ ID NO: 17) |
| | Antisense: 5′ AUGUUCUUUGAAUAAUUUCTT 3′ (SEQ ID NO: 18) |
| | Target: 5′ GAAAUUAUUCAAAGAACAU 3′ (SEQ ID NO: 30) |
| siSUV420H2#1 | Sense: 5′ GGAUCUGAGCCCUGACCCUTT 3′ (SEQ ID NO: 19) |
| | Antisense: 5′ AGGGUCAGGGCUCAGAUCCTT 3′ (SEQ ID NO: 20) |
| | Target: 5′ GGAUCUGAGCCCUGACCCU 3′ (SEQ ID NO: 31) |
| siSUV420H2#2 | Sense: 5′ GCAUAGCUCUGACCCUGGATT 3′ (SEQ ID NO: 21) |
| | Antisense: 5′ UCCAGGGUCAGAGCUAUGCTT 3′ (SEQ ID NO: 22) |
| | Target: 5′ GCAUAGCUCUGACCCUGGA 3′ (SEQ ID NO: 32) |

Construction of stable cell lines, constitutively expressing SUV420H2. V5-tagged SUV420H2 expression vectors (pcDNA5/FRT/V5-H1s-SUV420H2) were prepared and transfected those into Flp-In T-REx 293 cells (Invitrogen), which contains a Flp recombination target (FRT) site in its genome to express SUV420H2 conditionally and stably. V5-tagged chloramphenicol acetyltransferase (CAT) expression vectors (pcDNA5/FRT/V5-His-CAT) were used as a negative control for the experiments. SUV420H2 expression at the protein level was evaluated by Western blot and immunocytochemistry.

Flow cytometry assays (FACS) for cell cycle analysis. A549 cells treated with SUV420-specific siRNAs were prepared and cultured in a CO₂ incubator at 37 degrees C. for 72 hours. Then the cells after trypsin treatment were collected, and washed twice with 1,000 microliters of Assay Buffer and centrifuged for 5 min at 5,000 rpm. Then the supernatant was discarded, 200 microliter of Assay Buffer and 1,000 microliter of fixative buffer was added and the sample was incubated at room temperature for 1 hour. Finally, the propidium iodide reagent was added and cell cycle profiles were analyzed by flow cytometer (Cell Lab Quanta SC, Beckman Coulter). The proportion of each cell division was calculated and analyzed statistically by using Student's T test.

Coupled cell cycle and cell proliferation assay. A 5′-bromo-2′-deoxyuridine (BrdU) flow kit (BD Pharmingen, San Diego, Calif.) was used to determine the cell cycle kinetics and to measure the incorporation of BrdU into DNA of proliferating cells. The assay was performed according to the manufacture's protocol. Briefly, cells (2×10⁵ per well) were seeded overnight in 6-well tissue culture plates and treated with an optimized concentration of siRNAs in medium containing 10% FBS for 72 hours, followed by addition of 10 microM BrdU, and incubations continued for an additional 30 min. Both floating and adherent cells were pooled from triplicates wells per treatment point, fixed in a solution containing paraformaldehyde and the detergent saponin, and incubated for 1 hour with DNAase at 37 degrees C. (30 microgram per sample). FITC-conjugated anti-BrdU antibody (1:50 dilution in Wash buffer; BD Pharmingen, San Diego, Calif.) was added and incubation continued for 20 minutes at room temperature. Cells were washed in Wash buffer and total DNA was stained with 7-amino-actinomycin D (7-AAD; 20 microL per sample), followed by flow cytometric analysis using FACScan (BECKMAN COULTER) and total DNA content (7-AAD) was determined CXP Analysis Software Ver2.2 (BECKMAN COULTER).

Apoptosis assay. Apoptosis assay was performed by Western blotting and TUNEL assay. A549 and SBC5 cells were prepared, followed by siEGFP and siSUV420H2 treatments and incubated for 72 hours at 37 degrees C. Whole cell lysates were extracted by RIPA-like buffer, and Western Blotting was performed using alpha-PARP-1 antibody (F-2, mouse monoclonal, sc-8007, Santa Cruz), alpha-Cleaved Caspase-3 antibody (Asp175, rabbit polyclonal, Cell Signaling Technology) and alpha-GAPDH antibody (V-18, mouse monoclonal, sc-20357, Santa Cruz) as an internal control. TUNEL assay was performed by ApopTag™ Fluorescein Direct In Situ Apoptosis Detection Kit (CHEMICON-Millipore, Billerica, Mass.) according to the manufacturer's protocol. Cells were fixed at density of 1×10⁶ cells in 20 microlitter of 1% paraformaldehyde (pH 7.4) for 10 min at room temperature. Subsequently, 75 microlitter of equilibration buffer was applied and reacted for 10 sec, followed by applying working strength TdT enzyme in a humidified chamber at 37 degrees C. for 1 hour. This reaction was stopped by 55 microliter of Stop/Wash buffer. Apoptotic cells were stained by Alexa Fluor™ 488 and nuclei were counterstained by Propidium Iodide; Alexa Fluor™ 594.

### Example 2

### SUV420H1/H2 Expressions are Up-Regulated in Clinical Cancer Tissues

When the expression levels of various histone methyltransferases in a small subset of British clinical bladder cancer samples were examined, significant overexpression of SUV420H1 and SUV420H2 in the cancer samples compared with non-cancerous samples was found (data not shown). Subsequently, 124 bladder cancer samples and 24 normal control samples (British) were analyzed, and significant elevation of SUV420H1 and SUV420H2 expression levels in tumor cells compared with normal cells was confirmed (FIG. 1A). Subclassification of tumors according to gender, smoking history, grading, metastasis and recurrence identified no significant difference in their expression levels (Table 3). Then, the expression patterns of SUV420H1 and SUV420H2 in a number of Japanese clinical bladder cancer samples were analyzed by cDNA microarray, and significant overexpression in bladder cancers of Japanese patients was confirmed (FIG. 1B). In addition, previous microarray expression analysis of a large number of clinical samples [Kikuchi T, et al., Oncogene2003; 22:2192-2205, Nakamura T, et al., Oncogene 2004; 23:2385-2400, Nishidate T, et al., Int J Oncol 2004; 25:797-819, Takata R, et al., Clin Cancer Res 2005; 11:2625-2636.] indicated that both SUV420H1 and SUV420H2 expressions were significantly up-regulated in various types of cancer (FIG. 2 and Table 4). These results show that dysregulation of SUV420H1 and SUV420H2 expression can be involved in many types of human cancer.

TABLE 3
Statistical analysis of SUV420H1 and SUV420H2 expression levels in clinical bladder tissues.

| | | SUV420H1 | | | SUV420H2 | | |
|---|---|---|---|---|---|---|---|
| Factor | Case (n) | Mean | SD | 95% CI | Mean | SD | 95% CI |
| Normal (Control) | 24 | 2.352 | 0.618 | 2.091-2.613 | 1.472 | 1.501 | 0.838-2.106 |
| Tumor (Total) | 124 | 8.187 | 11.429 | 6.156-10.219 | 9.846 | 17.885 | 6.667-13.025 |
| Gender | | | | | | | |
| Male | 91 | 8.929 | 12.879 | 6.231-11.656 | 8.847 | 13.883 | 5.956-11.738 |
| Female | 31 | 5.422 | 4.435 | 3.795-7.049 | 7.629 | 17.331 | 1.258-14.396 |
| Smoke | | | | | | | |
| No | 27 | 11.623 | 17.862 | 4.557-18.689 | 6.004 | 7.356 | 3.095-8.914 |
| Yes | 49 | 7.793 | 11.28 | 4.553-11.033 | 10.369 | 19.725 | 4.704-16.035 |
| Grading | | | | | | | |
| G1 | 12 | 8.624 | 5.908 | 4.871-12.378 | 7.057 | 7.883 | 2.049-12.066 |
| G2 | 62 | 8.176 | 9.62 | 5.734-10.619 | 12.6 | 21.5 | 7.144-18.086 |
| G3 | 49 | 8.107 | 14.461 | 3.953-12.260 | 6.608 | 13.577 | 2.708-10.508 |
| Metastasis | | | | | | | |
| Negative | 97 | 8.68 | 12.347 | 6.130-11.366 | 10.225 | 18.603 | 6.476-13.975 |
| Positive | 27 | 6.419 | 7.135 | 3.597-9.242 | 8.482 | 15.26 | 2.446-14.519 |
| Recurrence | | | | | | | |
| No | 27 | 11.039 | 14.182 | 5.429-16.649 | 12.917 | 17.488 | 5.999-19.835 |
| Yes | 51 | 6.875 | 6.640 | 5.008-8.743 | 6.668 | 12.8 | 3.068-10.269 |
| Died | 8 | 5.539 | 6.733 | 1.470-12.570 | 13.667 | 28.487 | −10.148-37.483 |

TABLE 4
Gene expression profile of SUV420H1 and SUV420H2 in cancer tissues analyzed by cDNA
microarray*.

| | | Ratio (Tumor/Normal) | | | |
|---|---|---|---|---|---|
| | Case (n) | Count >2 | Count >3 | Count >5 | Count >10 |
| SUV420H1 | | | | | |
| Tissue type | | | | | |
| Bladder cancer | 34 | 22 (64.7%) | 12 (35.3%) | 4 (11.8%) | 0 (0%) |
| Cervical cancer | 19 | 17 (89.5%) | 10 (52.6%) | 4 (21.1%) | 1 (5.3%) |
| Osteosarcoma | 26 | 21 (80.8%) | 17 (65.4%) | 8 (30.8%) | 2 (7.7%) |
| Small cell lung cancer | 15 | 9 (60.0%) | 4 (26.7%) | 1 (6.7%) | 0 (0%) |
| Soft tissue tumor | 55 | 22 (44.0%) | 10 (18.2%) | 1 (6.7%) | 0 (0%) |
| SUV420H2 | | | | | |
| Tissue type | | | | | |
| Bladder cancer | 5 | 5 (100%) | 5 (100%) | 3 (60.0%) | 2 (40.0%) |
| Breast cancer | 6 | 4 (66.7%) | 4 (66.7%) | 3 (50.0%) | 1 (16.7%) |
| Chronic myelogenous leukemia | 10 | 8 (80.0%) | 6 (60.0%) | 2 (20.0%) | 1 (10.0%) |
| Esophageal cancer | 36 | 23 (63.9%) | 12 (33.3%) | 9 (25.0%) | 3 (8.3%) |
| Gastric cancer | 7 | 6 (85.7%) | 4 (57.1%) | 2 (28.6%) | 1 (14.3%) |
| Non-small cell lung cancer | 7 | 7 (100%) | 5 (71.4%) | 3 (42.9%) | 1 (14.3%) |
| Small cell lung cancer | 14 | 14 (100%) | 12 (85.7%) | 9 (64.3%) | 2 (14.3%) |

*The signal intensity of SUV420H1 and SUV420H2 between tumor tissues and corresponding non-neoplatic tissues derived from the same patient was compared.

### Example 3

### Association of SUV420H2 Expressions with Poor Prognosis for NSCLC Patients

Next, the present inventors compared SUV420H2 expression among bladder tumor tissues and various types of normal tissues and found that expression levels of SUV420H2 in bladder tumor tissues are significantly higher than those in normal tissues (FIG. 1C). Consistently, SUV420H2 expression in bladder cancer cell lines is notably high compared with that in normal cell lines (FIG. 3B).

cDNA microarray experiments showed that SUV420H2 expression was also elevated in lung tumor tissues compared with corresponding non-neoplastic tissues (FIG. 2B). To analyze the significance of SUV420H2 protein expression in lung cancer tissues in more detail, the present inventors conducted immunohistochemical analysis on a tissue microarray containing tissue sections from 340 NSCLC patients, who had undergone surgical resection (FIG. 2C). SUV420H2 stained positively in 295 out of 340 cases (86.8%) and negatively in 45 cases (13.2%). Subsequently, the present inventors analyzed the association of SUV420H2 expression with clinical outcome, and found that expression of SUV420H2 in NSCLC patients was significantly associated with male gender (P=0.0305, Fisher's exact test; Table 5) and tumor-specific 5-year survival after the resection of primary tumors (P=0.0023 bp log-rank test; FIG. 2D). Univariate analysis revealed associations between poor prognosis in NSCLC patients and several factors, including SUV420H2 expression, age, gender, histologic type (non-ADC versus ADC), pT stage (tumor size, T1 versus T2+T3) and pN stage (node status, N0 versus N1+N2, Table 6). In addition, multivariate analysis also revealed that SUV420H2 status shows statistical significance as an independent prognostic factor for surgically treated NSCLC patients enrolled in this study, as well as age, pT and pN factors (Table 6).

These results imply that deregulation of SUV420H2 expressions can be involved in many types of human cancer and correlated with a negative outcome in patients with NSCLC after surgical resection.

TABLE 5
Association between SUV420H2-positivity in NSCLC tissues and patients'
characteristics (n = 340).

| | | SUV420H2 expression | | | P value |
|---|---|---|---|---|---|
| | | Strong | Low | Absent | Strong |
| | Total | expression | expression | expression | vs Low |
| | n = 340 | n = 141 | n = 154 | n = 45 | or absent |
| Gender | | | | | |
| Female | 102 | 33 | 52 | 17 | 0.0305* |
| Male | 238 | 108 | 102 | 28 | |
| Age (year) | | | | | |
| <65 | 158 | 64 | 72 | 22 | 0.7421 |
| >=65 | 182 | 77 | 82 | 23 | |
| Smoking status | | | | | |
| never smoker | 96 | 34 | 48 | 14 | 0.1790 |
| current or | 244 | 107 | 106 | 31 | |
| ex-smoker | | | | | |
| Histological | | | | | |
| type | | | | | |
| ADC | 207 | 81 | 89 | 37 | 0.3103 |
| non-ADC | 133 | 60 | 65 | 8 | |
| T factor | | | | | |
| T1 | 143 | 58 | 64 | 21 | 0.8238 |
| T2 + T3 | 197 | 83 | 90 | 24 | |
| N factor | | | | | |
| N0 | 223 | 87 | 106 | 30 | 0.2466 |
| N1 + N2 | 117 | 54 | 48 | 15 | |

*P < 0.05 (Fisher's exact test)
ADC, adenocarcinoma
non-ADC, squamouse cell carcinoma plus large cell carcinoma and adenosquamous cell carcinoma

TABLE 6
Cox's Proportional Hazards Model Analysis of Prognostic Factors in
Patients with NSCLCs.

| | Hazards | | Unfavorable/ | |
|---|---|---|---|---|
| Variables | ratio | 95% CI | Favorable | P-value |
| Univariate | | | | |
| analysis | | | | |
| SUV420H2 | 1.677 | 1.199-2.346 | Positive/Negative | 0.0025* |
| Age (years) | 1.631 | 1.152-2.311 | >=65/65 > | 0.0058* |
| Gender | 1.559 | 1.054-2.307 | Male/Female | 0.0263* |
| Smoking status | 1.272 | 0.863-1.874 | Current or | 0.2245 |
| | | | ex-smoker/ | |
| | | | never smoker | |
| Histological type | 1.503 | 1.074-2.102 | non-ADC/ADC | 0.0173* |
| pT factor | 2.529 | 1.722-3.716 | T2 + T3/T1 | <0.0001* |
| pN factor | 2.220 | 1.586-3.105 | N1 + N2/N0 | <0.0001* |
| Multivariate | | | | |
| analysis | | | | |
| SUV420H2 | 1.624 | 1.156-2.281 | Positive/Negative | 0.0051* |
| Age (years) | 1.811 | 1.270-2.583 | >=65/65 > | 0.0010* |
| Gender | 1.148 | 0.735-1.793 | Male/Female | 0.5451 |
| Histological type | 1.027 | 0.700-1.506 | non-ADC/ADC | 0.8907 |
| pT factor | 2.113 | 1.406-3.176 | T2 + T3/T1 | 0.0003* |
| pN factor | 2.048 | 1.441-2.911 | N1 + N2/N0 | <0.0001* |

ADC, adenocarcinoma
non-ADC, squamous-cell carcinoma plus large-cell carcinoma and adenosquamous-cell carcinoma
*P < 0.05

### Example 4

### Growth Regulation of Cancer Cells by SUV420H1 and SUV420H2

To investigate roles of SUV420H1/H2 in human carcinogenesis, a knockdown experiment was performed using two independent siRNAs targeting SUV420H1 (siSUV420H1#1 and #2) and SUV420H2 (siSUV420H2#1 and #2). Firstly, the SUV420H1 and SUV420H2 expressions in various bladder and lung cancer cell lines were examined, and compared to the expression level in normal cell lines (FIGS. 3A and 3B). Expression levels of SUV420H1 and SUV420H2 in bladder and lung cancer cell lines were significantly higher than those in normal cell lines. In addition, SUV420H1 and SUV420H2 expressions in A549 and SBC5 cells transfected with siSUV420H1s and siSUV420H2s were significantly suppressed, compared with those transfected with control siRNA, siEGFP (FIGS. 3C and 3D). Using the same siRNAs, cell growth assays in two bladder cancer cell lines (SW780, RT4) and three lung cancer cell lines (A549, LC319 and SBC5) were performed, and significant growth suppression by the siSUV420H1s and siSUV420H2s was found, (FIGS. 4A and 4B). Growth suppression of cancer cells after knockdown of SUV420H2 was also confirmed by colony formation assay (FIG. 4E). To further assess the mechanism of growth suppression induced by the siRNA, the present inventors analyzed the cell cycle status of cancer cells after treatment with siRNAs using flow cytometry (FIG. 4C). The proportion of cancer cells at the sub-G₁ phase was significantly higher in the cells treated with siSUV420H2 than those treated with control siRNAs (FIG. 4C lower panel). This result was also confirmed by the other FACS analysis stained with FITC-BrdU and 7-AAD (FIG. 4F). These data imply that knockdown of SUV420H2 appears to induce apoptosis of cancer cells. In order to validate the apoptosis induction by knockdown of SUV420H2 in more detail, the present inventors conducted an apoptosis assay to monitor the cleavage of PARP1 and Caspase 3. As shown in FIG. 5A, cleaved types of PARP1 and Caspase 3 were observed after treatment with siSUV420H2. Moreover, TUNEL assay showed the DNA fragmentation of cancer cells after knockdown of SUV420H2 (FIG. 5B). These results reveal that apoptosis may be induced after treatment with the siSUV420H2.

To elucidate the mechanism for how SUV420H2 up-regulation influences the growth of cancer cells, the effect of SUV420H2 overexpression was examined using human embryonic kidney fibroblast (HEK293) cells containing the Flp-In T-REx system (T-REx-293, Invitrogen). The cell cycle status was analyzed by FACS analysis (FIG. 4D) and it was found that the proportions at the S phase were significantly increased in the T-REx-SUV420H2 cells compared with those in the control cells. These results show that SUV420H1/H2 expressions play important roles in the growth of cancer cells through enhancement of the cell cycle progression, and that inhibition of SUV420H2 can induce sub-G₁ phase of cancer cells.

The results illustrated the relationship between the SUV420H class of histone methyltransferase and human carcinogenesis and demonstrated that these enzymes could be ideal therapeutic targets in various types of malignancies.

### INDUSTRIAL APPLICABILITY

The present inventors have shown that the cell growth is suppressed by double-stranded nucleic acid molecules that specifically targets the SUV420H1 gene or the SUV420H2 gene. Thus, the double-stranded nucleic acid molecules targeting the SUV420H1 gene or the SUV420H2 gene are useful for the development of anti-cancer pharmaceuticals.

The expressions of SUV420H1 gene and SUV420H2 gene are markedly elevated in bladder cancer, cervical cancer, osteosarcoma, lung cancer, soft tissue tumor, breast cancer, chronic myelogenous leukemia (CML), esophageal cancer and gastric cancer. In particular, it was confirmed that the SUV420H1 gene was markedly elevated in bladder cancer, cervical cancer, osteosarcoma, lung cancer and soft tissue tumor, and the SUV420H2 was markedly elevated in bladder cancer, breast cancer, chronic myelogenous leukemia (CML), esophageal cancer, gastric cancer and lung cancer.

Accordingly, these gene markers can be conveniently used as diagnostic markers of cancers and the mRNAs and the proteins encoded thereby may be used in diagnostic assays of cancers.

Furthermore, as described herein, the expression of the SUV420H2 gene is associated with poor prognosis in patients with NSCLC. Therefore, the present invention also provides a novel prognostic marker, SUV420H2.

Furthermore, the SUV420H1 and SUV420H2 polypeptides are useful targets for the development of anti-cancer pharmaceuticals. For example, substances that bind SUV420H1 or SUV420H2 or block the expression of SUV420H1 or SUV420H2, or inhibit the biological activity of SUV420H1 or SUV420H2 may find therapeutic utility as anti-cancer agents, particularly anti-cancer agents for the treatment of bladder cancer, cervical cancer, osteosarcoma, lung cancer, soft tissue tumor, breast cancer, chronic myelogenous leukemia (CML), esophageal cancer or gastric cancer.

While the invention has been described in detail and with reference to specific embodiments thereof, it will be apparent to one skilled in the art that various changes and modifications can be made therein without departing from the spirit and scope of the invention. All publications, patent applications, patents, and other references mentioned herein are incorporated by reference in their entirety. In case of conflict, the present specification, including definitions, will control. In addition, the materials, methods, and examples are illustrative only and not intended to be limiting.

1.-6. (canceled)

7. A method of screening for a candidate substance for treating or preventing cancer, or inhibiting cancer cell growth, said method comprising the steps of:
(a) contacting a test substance with an SUV420H1 or SUV420H2 polypeptide;
(b) detecting the binding activity between the polypeptide and the test substance; and
(c) selecting the test substance that binds to the polypeptide.

8. A method of screening for a candidate substance for treating or preventing cancer or inhibiting cancer cell growth, said method comprising the steps of:
(a) contacting a test substance with a cell expressing an SUV420H1 or SUV420H2 gene;
(b) detecting the expression level of the SUV420H1 or SUV420H2 gene in the cell; and
(c) selecting the test substance that reduces the expression level of the SUV420H1 or SUV420H2 gene in comparison with the expression level in the absence of the test substance.

9. A method of screening for a candidate substance for treating or preventing cancer or inhibiting cancer cell growth, said method comprising the steps of:
(a) contacting a test substance with an SUV420H1 or SUV420H2 polypeptide;
(b) detecting a biological activity of the polypeptide of step (a); and
(c) selecting the test substance that suppresses the biological activity of the polypeptide in comparison with the biological activity detected in the absence of the test substance.

10. The method of claim 9, wherein the biological activity is cell proliferation enhancing activity, methyltransferase activity or anti-apoptotic activity.

11. A method of screening for a candidate substance for treating or preventing cancer or inhibiting cancer cell growth, said method comprising the steps of:
(a) contacting a test substance with a cell into which a vector comprising the transcriptional regulatory region of an SUV420H1 or SUV420H2 gene and a reporter gene that is expressed under the control of the transcriptional regulatory region has been introduced,
(b) measuring the expression or activity of said reporter gene; and
(c) selecting the test substance that reduces the expression or activity level of said reporter gene, in comparison with the level in the absence of the test substance.

12.-28. (canceled)
